SEQUENCE LISTING (10 sequences)

SEQ ID NO: 1 - DNA, 1547 bp, Bacillus velezensis
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 2 - DNA, 1289 bp, Bacillus licheniformis
  source 1..1289: mol_type = genomic DNA
gtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 3 - DNA, 1567 bp, Bacillus thuringiensis
  source 1..1567: mol_type = genomic DNA
aaaggaggtgatccagccgcaccttccgatacggctaccttgttacgacttcaccccaatcatctgtcccaccttaggcggctggctccataaaggttaccccaccgacttcgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaacggttttatgagattagctccacctcgcggtcttgcagctctttgtaccgtccattgtagcacgtgtgtagcccaggtcataaggggcatgatgatttgacgtcatccccaccttcctccggtttgtcaccggcagtcaccttagagtgcccaacttaatgatggcaactaagatcaagggttgcgctcgttgcgggacttaacccaacatctcacgacacgagctgacgacacgacgaagctgacgacaaccatgcaccacctgtcactctgctcccgaaggagaagccctatctctagggttttcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttaacttcagcactaaagggcggaaaccctctaacacttagcactcatcgtttacggcgtggactaccagggtatctaatcctgtttgctccccacgctttcgcgcctcagtgtcagttacagaccagaaagtcgccttcgccactggtgttcctccatatctctacgcatttcaccgctacacatggaattccactttcctcttctgcactcaagtctcccagtttccaatgaccctccacggttgagccgtgggctttcacatcagacttaagaaaccacctgcgcgcgctttacgcccaataattccggataacgcttgccacctacgtattaccgcggctgctggcacgtagttagccgtggctttctggttaggtaccgtcaaggtgccagcttattcaactagcacttgttcttccctaacaacagagttttacgacccgaaagccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgttgccttggtgagccgttacctcaccaactagctaatgcgacgcgggtccatccataagtgacagccaaagccgcctttcaatttcgaaccatgcggttcaaaatgttatccggtattagccccggtttcccggagttatcccagtcttatgggcaggttacccacgtgttactcacccgtccgccgctaacttcttgagagcaagctctcaatccattcgctcgacttgcatgtattaggcacgccgccagcgttcatcctgagccaggatcaaactctccaat

SEQ ID NO: 4 - DNA, 1408 bp, Paenibacillus polymyxa
  source 1..1408: mol_type = genomic DNA
tagaagcttgcttctaaataatctagcggcggacgggtgagtaacacgtaggcaacctgcccacaagacagggataactaccggaaacggtagctaatacccgatacatccttttcctgcatgggagaaggaggaaagacggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatcccgaggctcaacttcgggtcgcactggaaactggagagcttgagtgcagaagaggagagtggaattccacgtgtagcggtgaaatgcgtagagatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggngccagccgccgaaggt

SEQ ID NO: 5 - DNA, 1390 bp, Paenibacillus peoriae
  source 1..1390: mol_type = genomic DNA
cttcggcggctggctcccttgcgggttaccccaccgacttcgggtgttgtaaactctcgtggtgtgacgggcggtgtgtacaagacccgggaacgtattcaccgcggcatgctgatccgcgattactagcaattccgacttcatgtaggcgagttgcagcctacaatccgaactgagaccggcttttctaggattggctccacctcgcgatttcgcttcccgttgtaccggccattgtagtacgtgtgtagcccaggtcataaggggcatgatgatttgacgtcatccccaccttcctccggtttgtcaccggcagtctgcttagagtgcccagcttgacctgctggcaactaagcataagggttgcgctcgttgcgggacttaacccaacatctcacgacacgagctgacgacaaccatgcaccacctgtctcctctgtcccgaaggaaaggtctatctctagaccggtcagagggatgtcaagacctggtaaggttcttcgcgttgcttcgaattaaaccacatactccactgcttgtgcgggtccccgtcaattcctttgagtttcagtcttgcgaccgtactccccaggcggaatgcttaatgtgttaacttcggcaccaagggtatcgaaacccctaacacctagcattcatcgtttacggcgtggactaccagggtatctaatcctgtttgctccccacgctttcgcgcctcagcgtcagttacagcccagagagtcgccttcgccactggtgttcctccacatctctacgcatttcaccgctacacgtggaattccactctcctcttctgcactcaagctccccagtttccagtgcgacccgaagttgagcctcgggattaaacaccagacttaaagagccgcctgcgcgcgctttacgcccaataattccggacaacgcttgccccctacgtattaccgcggctgctggcacgtagttagccggggctttcttctcaggtaccgtcactcttgtagcagttactctacaagacgttcttccctggcaacagagctttacgatccgaaaaccttcatcactcacgcggcgttgctccgtcaggctttcgcccattgcggaagattccctactgctgcctcccgtaggagtctgggccgtgtctcagtcccagtgtggccgatcaccctctcaggtcggctacgcatcgtcgccttggtaggcctttaccccaccaactagctaatgcgccgcaggcccatccacaagtgacagattgctccgtctttcctccttctcccatgcaggaaaaggatgtatcgggtattagctaccgtttccggtagttatccctgtcttgtgggcaggttgcctacgtgttactcacccgtccgccgctaggtnn

SEQ ID NO: 6 - DNA, 1397 bp, Bacillus velezensis
  source 1..1397: mol_type = genomic DNA
agtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacc

SEQ ID NO: 7 - DNA, 1489 bp, Bacillus pumilus
  source 1..1489: mol_type = genomic DNA
gtcgagcgaacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaaggtagccgtatcggaaggtgcggctggatcacctccttt

SEQ ID NO: 8 - DNA, 1821 bp, Bacillus velezensis
  source 1..1821: mol_type = genomic DNA
gtggaacaggatactcagcatgtcaaacagtttcaatcgaaaaccgatcttcatgccgttttgacgtctaacgggcgtattatgtatatttctgcaaactgcaaatcgtacttcagttattctcaggaagagatgatcgggtcatttctcaaaacgttcctgcatgaagaagatcaatttttggttgaaagttacttctataatgagcatcatttgatgccttgcacctttcgtcttattaaaaaagatcataccatcgtatggcttgaagcgggtgtggaaattgtcacaccgcaaatggaggggacggatcgggaaatcatcctcaaaatcaaggtgcttgaagagaagacgccccgccggccttatatgtatgaacaaaagaagcaaaaggcagaacaacagtcggaagatgcggctggaaacggcgaattcgagcgtcttgtcgaaatgctgccgaatcctttatatgtcagcatcaacggcacaatcgcatacgccaacaacgcgatgcttgccatgctgggagccgaaagcaaagacgaggtaatcggcaggcgctcttatgagtttattgaagaagagtatcacgacattgtgaaaaaccgaattgtgcggatgcaaaaaggaatggaagtcggaatgatcgaacagacgtggagaaggctcgacggcacgcccgttcatttagaagtaaaggcggcaccgacgacatataaaaatcagcgggcggaattgctgcttctgattgatatttcttcacggaaaaaattccaaacgattctgcaaaaaagcagagagcgttatcagctcctcattcagaattcaattgatacgatcgccgtgattcacaatgataaatgggtattcatgaatgagtcgggcatttcgctgtttgaggctgaaacatatgaagatttaatcgggaaaaacgtctatgatcagcttcatccctgtgatcatgacggcgtgaaacagcggatccaaaatattatcaaccgaaagaccgagtcagaaattattaagcaatcgtggtttacctttaaaaaccggctgatctacacggaaatggtctgcattccgacgacgtttttcggagaaacggccgtgcaggtcattctgcgtgatatttctgagcggaagcagacggaggaattaatgctgaagtcggagaagctgtctatcgcagggcagctggcggcgggaatcgctcacgaaatccgcaatccgctgacggcgatcaaaggatttttacagctcatgaagccgacgatggaggaaaacgaacattactttgaaattgtgttttctgaactgagcagaatcgaattaattttaagtgagcttttaatgcttgccaaacctcagcaaaacgccgtgaaagagcgtgtaaacttaaagaaaattataagtgaagtgaccgctcttttagagacgcaggcaaatctgaaaggaatttttatcaaaacggattacgaacatgacagcatgtatattaacggtgatcaaaatcagctgaaacaggtgtttatcaatctgattaaaaatgccgttgagtcgatgccagatgggggaacggttcacattcttatgacagaagatgagtattccgttaatgtgacggtgaaagacgagggtgacgggataccggaaaacgtgctgaaacgaataggcgaaccgtttttgacaacgaaagaaaaaggaaccggcctggggcttatggtcacattcaaccttattaaaaaccatcagggagccatccaagtagacagcaaaccggatagaggcacggcctttcatattacattcccgaaacaataa

SEQ ID NO: 9 - DNA, 1283 bp, Paenibacillus polymyxa
  source 1..1283: mol_type = genomic DNA
ggttatggagcttatccgtgatttgttgattcaaattgccatcataattttaccactatttttatatgaagcgattcgtttaaatcgctatcaagaaatgcctccgaagccaaatcgttactttattatgtttttatctagcgttacgctcgttctctccatgacatactctatttgttttggcaatgtgtgtgggtataactttcacccaattccaattgtcagtggttttttatatggtgggattgttggtttaatcccagcggttatttttgttgcatatgaatggttactaaagggaatgagctttcttccagttttagaagtaacatttctattgatagttccgctatttttatcaaagaaatggtctcttttttcaagagataagaaattaattttagcatttatgatttcatcattttatgtgcttgtttctttagtattaggcatgattaatgttcttttggaaacaggatttacaccgtatatttcgcaaatatatagcggatttatatttgcctcgcttatcatggtcatgacaatggtatttcaggtgtatttaactgaatatttaaatgaaaatgcattgctacgtacagagatgcaaaaatcagaaaagcttaatattgtaagtgagttagcagcaagtgtggcacatgaagttcgaaatccacttactgttgttcgtggatttatccaattgctagaaagtacggaagatgtgaagaataaggattatatgcgtctcgtattagctgaattggatcgggcggaacaaattatttcagattacttgaatttagcaaggccgcaaattgagaaaaaagaacacatttgtttatcagcccaattaatagaaatgaccactcttatgtcgtcatttgcagcgatgcaaggtgtatatttacaagttgaaatttcagaaagtctttatacgattggtgataagacaaaattgaagcaagctattatgaatgttgttaaaaatggtattgaagcaattcaaggaaataaaggatatttaaaagtaacagctattcaaaaagacgaaacaatcataataagagttaaagatagtggtgttggtatgacgaaagaacagttagcaaggctggggcaaccatattattctttaaaagaaaaagggacaggattagggcttatggtaacatttagtattttacaagcgcataatggtacattggaatataagagtgaaagtggaaaaggcactgaagcaattattatattaccagctgtacaaaataaggaataaaagt

SEQ ID NO: 10 - DNA, 1821 bp, Bacillus velezensis
  source 1..1821: mol_type = genomic DNA
gtggaacaggatactcagcatgtcaaacagtttcaatcgaaaactgatcttcatgccgttttgacgtctaacgggcgtattatgtatatttctgcaaactgcaaatcgtacttcagttattctcaggaagagatgattgggtcatttctcaaaacgttcctgcatgaagaagatcaatttttggttgaaagttacttctataatgagcatcatttgatgccttgcacctttcgtcttattaaaaaagatcataccatcgtatggcttgaagcgggtgtggaaattgtcacaccgcaaatggaggggacggatcgggaaatcatcctcaaaatcaaggtgcttgaggagaagacgccccgccggccttatatgtatgaacaaaagaagcaaaaggcagaacaacagtcggaagatgcggctggaaacggcgaattcgagcgtcttgtcgaaatgctgccgaatcctttatatgtcagcatcaacggcacaatcgcatacgccaacaacgcgatgcttgccatgctgggagccgaaagcaaagacgaggtaatcggcaggcgctcttatgagtttattgaagaagagtatcacgacattgtgaaaaaccgaattgtgcggatgcaaaaaggaatggaagtcggaatgatcgaacagacgtggagaaggctcgacggcacgcccgttcatttagaagtaaaggcggcaccgacgacatataaaaatcagcgggcggaattgctgcttctgattgatatttcttcacgaaaaaaattccaaacgattctgcaaaaaagcagagagcgttatcagctcctcattcagaattcaattgatacgatcgccgtgattcacaatgataaatgggtattcatgaatgagtcgggcatttcgctgtttgaggctgaaacatatgaagatttaatcgggaaaaacgtctatgatcagcttcatccctgtgatcatgacggcgtgaaacagcggatccaaaatattatcaaccgaaagaccgagtcagaaattattaagcaatcgtggtttacctttaaaaaccggctgatctacacggaaatggtctgcattccgacgacgtttttcggagaaacggccgtgcaggtcattctgcgtgatatttctgagcggaagcagacggaggaattaatgctgaagtcggagaagctgtctatcgcagggcagctggcggcgggaatcgctcacgaaatccgcaatccgctgacggcgatcaaaggatttttacagctcatgaagccgacgatggaggaaaacgaacattactttgaaattgtgttttctgaactgagcagaatcgaattaattttaagtgagcttttaatgcttgccaaacctcagcaaaacgccgtgaaagagcgtgtaaacttaaagaaaattataagtgaagtgaccgctcttttagagacgcaggcaaatctgaaagggatttttatcaaaacggattacgaacatgacagcatgtatattaacggtgatcaaaatcagctgaaacaggtgtttatcaatctgattaaaaatgccgttgagtcgatgccagatgggggcacggttcacattcttatgacagaagatgagtattccgttaatgtgacggtgaaagacgagggtgacgggataccggaaaacgtgctgaaacgaataggcgaaccgtttttgacaacgaaagaaaaaggaaccggcctggggcttatggtcacattcaaccttattaaaaaccatcagggagccatccaagtagacagcaaaccggatagaggcacggcctttcatattacattcccgaaacaataa

(19) United States
(12) Patent Application Publication
(10) Pub. No.: US 2026/0226405 A1
(43) Pub. Date: Aug. 6, 2026

(54) GENOME EDITED MICROBES FOR IMPROVED FUNGICIDAL AND BACTERICIDAL ACTIVITY

(71) Applicant: BIOCONSORTIA, INC., DAVIS, CA (US)

(72) Inventors: MACKENZIE CARTER, SACRAMENTO, CA (US); BETSY ALFORD, DAVIS, CA (US); DAMIAN CURTIS, DAVIS, CA (US); ANDREW PHILLIPS, SACRAMENTO, CA (US); ABHISHEK PATRI, WEST SACRAMENTO, CA (US); FIDELE AKUM, DAVIS, CA (US)

(73) Assignee: BIOCONSORTIA, INC., DAVIS, CA (US)

(21) Appl. No.: 19/153,040

(22) PCT Filed: Feb. 9, 2024
(86) PCT No.: PCT/US2024/015231

§ 371 Date: Aug. 1, 2025

Related U.S. Application Data
(60) Provisional application No. 63/484,449, filed on Feb. 10, 2023.

(51) Int. Cl. C12N 1/20; A01N 63/22; A01N 63/25; A01P 1/00; A01P 3/00; C12N 9/12; C12N 9/88; C12N 15/63; C12R 1/07

(52) U.S. Cl. CPC C12N 1/20; A01N 63/22; A01N 63/25; A01P 1/00; A01P 3/00; C12N 9/12; C12N 9/88; C12N 15/63; C12R 2001/07; C12Y 207/13003; C12Y 402/03001

(57) ABSTRACT

The present disclosure relates to methods and compositions of genetically engineered microbes, for improved control of pathogens, such as bacteria and/or fungi, with application in various fields, such as pharmaceuticals and agriculture. The genetically engineered microbes may comprise a deletion or knockout at one or more genomic loci, that confers capability of reducing hyphal growth of a fungus, reduce spore formation of a fungus, and/or other benefits.

### CROSS-REFERENCE TO RELATED APPLICATIONS

This application claims the benefit under 35 U.S.C. 119(e) of U.S. Provisional Patent Application Ser. No. 63/484,449 filed 10 Feb. 2023, herein incorporated by reference in its entirety.

### REFERENCE TO SEQUENCE LISTING SUBMITTED ELECTRONICALLY

The official copy of the sequence listing is submitted electronically as a WIPO ST26 compliant XML sequence listing with a file named 23071-WO-PCT.xml created on 3 Feb. 2024 and having a size of 24,530 bytes and is filed concurrently with the specification. The sequence listing comprised in this document is part of the specification and is herein incorporated by reference in its entirety.

### FIELD

The present disclosure relates to methods and compositions of genetically modified microorganisms that produce metabolites, such as secondary metabolites, for example lipopeptides and/or polyketides, useful in agriculture and amongst other fields. The present disclosure further relates to compositions comprising the metabolites, or the microorganisms that produce the metabolites, and methods for identifying and using the same in agriculture and other fields of application.

### BACKGROUND

According to the United Nations World Food Program, there are close to 900 million malnourished people in the world. The malnourishment epidemic is particularly striking in the developing nations of the world, where one in six children is underweight. The paucity of available food can be attributed to many socioeconomic factors; however, regardless of ultimate cause, the fact remains that there is a shortage of food available to feed a growing world population, which is expected to reach 9 billion people by 2037. The United Nations estimates that agricultural yields must increase by 70-100% to feed the projected global population.

Many factors impact the health and production output of crop plants, including stresses introduced by other living organisms (biotic stresses) or by other factors (abiotic stresses, e.g., insufficient water, excess water, poor nutrient availability, toxicity due to salt, minerals, or other compounds).

Biotic stress in plants is caused by living organisms, especially viruses, bacteria, fungi, nematodes, insects, arachnids, and weeds. The agents cause biotic stress, which directly deprive their host of its nutrients, and can lead to death of plants.

Despite the advancements made by technological innovations such as genetically engineered crops and new novel pesticidal and herbicidal compounds, however, there remains a need for improved crop performance to meet the demands of an increasing global population.

Thus, novel compositions and methods are needed to improve the ability for crop plants to withstand biotic stress. Use of live microbes or microbial products for biocontrol of pests and diseases offers an attractive alternative to synthetic chemical pesticides and fungicides, due to reduced toxicity, and greater biodegradability resulting in fewer problems with environmental persistence. Biological compositions are also typically faster and less expensive to develop, making them commercially accessible within timeframes that enable benefits to growers and consumers.

### SUMMARY

The present disclosure addresses this important issue of how to improve crop performance, thereby closing the worldwide yield gap, along with providing ways of imparting other beneficial traits to plant species.

The solution to increasing crop performance and increasing yield proffered by the present disclosure is not detrimental to the earth's resources, as it does not rely upon increased water consumption or increased input of synthetic chemicals into a system. Rather, the present disclosure utilizes microbes, or metabolites produced by microbes, to impart beneficial properties, including increased yields, to desirable plants.

The disclosure therefore offers an environmentally sustainable solution that allows farmers to increase yields of important crops, particularly crops susceptible to one or more biotic stressors. Without limitation, biotic stressors can include pests such as nematodes, and phytopathogens such as fungi.

In one aspect, the present disclosure relates to a method of selecting a microorganism that produces one or more metabolites that impart one or more beneficial traits to a plant including: obtaining a first sample having one or more metabolites from a microorganism; obtaining a first metabolite profile from the first sample; and selecting the microorganism that produces metabolites that impart one or more beneficial traits to a plant when the first metabolite profile has one or more unique elements, wherein at least one of the one or more unique elements corresponds to the one or more metabolites that impart the one or more beneficial traits to the plant.

In some embodiments, the method of selecting a microorganism that produces one or more metabolites that impart one or more beneficial traits to a plant includes comparing the first metabolite profile to a second metabolite profile, wherein the second metabolite profile is obtained from a second sample having one or more metabolites from a second microorganism and the second microorganism does not produce metabolites that impart one or more beneficial traits to the plant.

In some embodiments, the one or more beneficial traits that are imparted to the plant include the control or biocontrol of phytopathogens. In some aspects, the phytopathogen causes damage to a plant before harvest. In some aspects, the phytopathogen causes damage to a harvested part of the plant (e.g., fruit, seed, lint, leaf).

In some aspects, the lipopeptide is a cyclic lipopeptide.

In some aspects, the cyclic lipopeptide is part of an assembly of lipopeptides. In some aspects, the assembly comprises a plurality of different lipopeptides. In some aspects, the assembly comprises a plurality of lipopeptides of the same kind.

In another aspect, the present disclosure relates to a composition having an isolated metabolite mixture, wherein the isolated metabolite mixture is derived from a microorganism selected via the methods disclosed herein. In some embodiments, the isolated metabolite mixtures have one or more lipopeptides.

In some embodiments, the present disclosure relates to a composition having an isolated metabolite mixture, wherein the isolated metabolite mixture is derived from a lipopeptide-producing microorganism.

In another aspect, the present disclosure relates to a composition having an isolated microorganism, wherein the isolated microorganism is selected via the methods disclosed herein. In some embodiments, the isolated microorganism produces a metabolite mixture having one or more lipopeptides.

In another aspect, the present disclosure relates to a method of imparting one or more beneficial traits to a plant including applying the compositions disclosed herein to the plant, or to a growth medium in which the plant is located. In some embodiments, the one or more beneficial traits imparted to the plant is the biocontrol of one or more pest(s) and/or phytopathogen(s).

In another aspect, the present disclosure relates to a method of creating variant strain(s) that produce(s) an improved amount or relative ratio of one or more metabolites that control one or more biotic stressors, for example a fungus.

In another aspect, the present disclosure relates to a composition having one or more isolated metabolites, wherein the one or more isolated metabolites are derived from a microorganism selected via the methods disclosed herein. In some embodiments, the isolated metabolites have one or more lipopeptides.

In some embodiments, the present disclosure relates to a composition having one or more isolated metabolites, wherein: the one or more metabolites are one or more lipopeptides derived from a genetically engineered strain.

In some embodiments, the composition is applied to the plant or plant part prior to germination. In some embodiments, the composition is applied to the plant or plant part pre-harvest. In some embodiments, the composition is applied to the plant or plant part during the vegetative phase of the plant. In some embodiments, the composition is applied to the plant or plant during the reproductive phase of the plant. In some embodiments, the composition is applied to the plant or plant part pre-harvest. In some embodiments, the composition is applied to the plant or plant part post-harvest. In some embodiments, the planted is cultivated in a field, harvested, placed in storage, or distributed. In some embodiments, the plant or a part thereof is harvested. In some embodiments, the plant produces an agriculturally-important fruit, grain, cereal, fiber, food, feed, fuel, or seed. In some embodiments, the parameter is selected from the group consisting of: presence of pathogen, distribution of pathogen, quantification of pathogen, type of pathogen, biological status of the pathogen, viability of the pathogen, presence of pest, distribution of pest, quantification of pest, type of pest, biological status of the pest, viability of the pest, visual assessment of the plant part, biomass of the plant part, biological status of the plant part, viability of the plant part, and any combination of the preceding. In some embodiments, the plant part is a root, leaf, stem, flower, seed, bulb, or fruit. In some embodiments, the method further comprising applying a plurality of compositions of (b) to the plant. In some embodiments, the method comprising applying a plurality of compositions in step (b). In some embodiments, the method comprising applying a plurality of compositions in step (b), wherein each of the plurality of compositions comprises a different characteristic of step (b), or any combination of the preceding. In some embodiments, the method further comprises applying a plurality of compositions in step (b), wherein each of the plurality of compositions comprises a different characteristic of step (b), or any combination of the preceding.

In some embodiments, the synthetic composition is substantially confined within an object selected from the group consisting of: bottle, jar, ampule, package, vessel, bag, box, bin, envelope, carton, container, silo, shipping container, truck bed, and case. In some embodiments, the synthetic composition of (b) is a cyclic lipopeptide. In some embodiments, the composition of (b) is derived from a genetically engineered strain. In some embodiments, the composition of (b) is substantially purified. In some embodiments, the synthetic composition further comprises one or more formulation component(s). In some embodiments, the one or more formulation component(s) is(are) selected from the group consisting of: a salt, a binder, a surface-active agent, a surfactant, a wetting agent, a dispersing agent, an emulsifying agent, a solubilizing agent, an organic solvent, a gelling agent, a thickening agent, an anti-settling agent, a preservative, a stabilizer, an anti-free compound, a plurality of any of the preceding, and any combination of the preceding. In some embodiments, the synthetic composition further comprises one or more additional agents selected from the group consisting of: a pesticide, an herbicide, a bactericide, a fungicide, an insecticide, a virucide, a miticide, a nematicide, an acaricide, a plant growth regulator, a rodenticide, an anti-algae agent, a biocontrol agent, a fertilizer, a biopesticide, a biostimulant, and any combination and/or plurality of the preceding. In some embodiments, the synthetic composition further comprises a fungus. In some embodiments, the plant or plant part of (a) produces an agriculturally-important fruit, grain, food, fuel, feed, cereal, fiber, or seed. In some embodiments, the synthetic composition further comprises a growth medium. In some embodiments, the plant part is a root, leaf, stem, flower, seed, bulb, or fruit. In some embodiments, a plurality of the synthetic compositions is provided. In some embodiments, each of the plurality of compositions comprises a different characteristic of step (b), or any combination or plurality of the preceding.

In some aspects, the synthetic composition may comprise a plurality or combination of same or different microbes and/or lipopeptides.

In some aspects, a method of producing a composition that improves the biotic stress tolerance of a plant is provided, the method comprising: obtaining a microbe, and culturing said microbe under conditions suitable for growth and reproduction; and identifying a composition of the microbe.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1A shows the cyclic lipopeptide (CLP) HPLC peak areas (Iturins, Fengycins, Surfactins, and total CLPs) for whole cell broths obtained from Wild Type Strain 7084 (7084-F), as compared to genetically modified strains 7084-G77, 7084-G93, and 7084-G105.

FIG. 1B shows the cyclic lipopeptide (CLP) HPLC peak areas (Iturins, Fengycins, Surfactins, and total CLPs) for duplicate runs (−1 and −2, respectively) for whole cell broths obtained from Wild Type Strain 7084 (7084-F), as compared to genetically modified strains 7084-G103, 7084-G77, 7084-G95, and 7084-G97.

FIG. 2A shows the polyketide HPLC peak areas (Macrolactins, Difficidins) for supernatant material obtained from Wild Type Strain 7084 (7084-F), as compared to genetically modified strains 7084-G77, 7084-G93, and 7084-G105.

FIG. 2B shows the polyketide HPLC peak areas (Difficidin 1, Difficidin 2, Difficidin 3) for duplicate runs (−1 and −2, respectively) for supernatant material obtained from Wild Type Strain 7084 (7084-F), as compared to genetically modified strains 7084-G103, 7084-G77, 7084-G95, and 7084-G97.

FIG. 2C shows the macrolactin HPLC peak areas (Macrolactin 1, Macrolactin 2, Macrolactin 3, Macrolactin 4, Macrolactin 5) for duplicate runs (−1 and −2, respectively) for supernatant material obtained from Wild Type Strain 7084 (7084-F), as compared to genetically modified strains 7084-G103, 7084-G77, 7084-G95, and 7084-G97.

FIG. 2D shows the baccilaene HPLC peak areas for duplicate runs (−1 and −2, respectively) for supernatant material obtained from Wild Type Strain 7084 (7084-F), as compared to genetically modified strains 7084-G103, 7084-G77, 7084-G95, and 7084-G97.

FIG. 3A is the HPLC chromatogram for two different producing variants of genetically modified strain 7084-G67 (magenta line=Variant 1/“high producer”, royal blue line=Variant 2/“low producer”) as compared to that of Wild Type Strain 7084 (black line). The cyan lines indicate the peak start/stop for peak area integration.

FIG. 3B is a close-up of part of the 7084-G67 Variant 2 HPLC chromatogram (royal blue line), showing (royal blue arrow) a shift in retention time of one of the Surfactins, as compared to that of the Wild Type Strain 7084 (7084-F) (black line).

FIG. 3C shows relative production of cyclic lipopeptides of 7084 (WT, far right), the 7084-G67 low producing variant (Variant 2, far left), and the 7084-G67 high producing variant (Variant 1, middle).

FIG. 3D shows relative production of polyketides of 7084 (WT, far right), the 7084-G67 low producing variant (Variant 2, far left), and the 7084-G67 high producing variant (Variant 1, middle).

FIG. 4 is the HPLC chromatogram for genetically modified strain 7084-G77 (magenta line) as compared to that of Wild Type Strain 7084 (black line). The cyan lines indicate the peak start/stop for peak area integration.

FIG. 5 is the HPLC chromatogram for genetically modified strain 7084-G93 (brown line) as compared to that of Wild Type Strain 7084 (black line). The cyan lines indicate the peak start/stop for peak area integration.

FIG. 6 is the HPLC chromatogram for genetically modified strain 7084-G105 (green line) as compared to that of Wild Type Strain 7084 (black line). The cyan lines indicate the peak start/stop for peak area integration.

FIG. 7 is the HPLC chromatogram for genetically modified strain 7084-G68 (black line). The cyan lines indicate the peak start/stop for peak area integration.

FIG. 8 is the HPLC chromatogram for genetically modified strain 7084-G75 (black line). The cyan lines indicate the peak start/stop for peak area integration.

FIG. 9 is the HPLC chromatogram for genetically modified strain 7084-G76 (black line). The cyan lines indicate the peak start/stop for peak area integration.

FIG. 10 is the HPLC chromatogram for genetically modified strain 7084-G95 (royal blue line). The cyan lines indicate the peak start/stop for peak area integration.

FIG. 11 is the HPLC chromatogram for genetically modified strain 7084-G97 (magenta line). The cyan lines indicate the peak start/stop for peak area integration.

FIG. 12 is the HPLC chromatogram for genetically modified strain 7084-G103 (brown line). The cyan lines indicate the peak start/stop for peak area integration.

FIG. 13A shows the antifungal activity of Whole Cell Broths (WCB) obtained from Wild Type Strain 7084 and genetically modified strain 7084-G77 at different dilutions against Fusarium gramminearum.

FIG. 13B shows photographs of cell culture plates of Wild Type Strain 7084 and genetically modified strain 7084-G77 at different dilutions against Fusarium gramminearum, as compared to an untreated control inoculated with F. gramminearum.

FIG. 14A shows the antifungal activity of Whole Cell Broths (WCB) obtained from Wild Type Strain 7084 and genetically modified strain 7084-G77 at different dilutions against Penicillium expansum.

FIG. 14B shows photographs of cell culture plates of Wild Type Strain 7084 and genetically modified strain 7084-G77 at 1:50 dilutions against Penicillium expansum, as compared to an untreated control inoculated with Penicillium expansum.

FIG. 15A shows the antifungal activity of Whole Cell Broths (WCB) obtained from Wild Type Strain 7084 and genetically modified strain 7084-G77 at different dilutions against Fusarium oxysporum f.sp. lycopersici.

FIG. 15B shows photographs of cell culture plates of Wild Type Strain 7084 and genetically modified strain 7084-G77 at different dilutions against Fusarium oxysporum f.sp. lycopersici, as compared to an untreated control inoculated with Fusarium oxysporum f.sp. lycopersici.

FIG. 16A shows the antifungal activity of Whole Cell Broths (WCB) obtained from Wild Type Strain 7084 and genetically modified strain 7084-G77 at different dilutions against Fusarium oxysporum f.sp. lactucae.

FIG. 16B shows photographs of cell culture plates of Wild Type Strain 7084 and genetically modified strain 7084-G77 (3 dpi) at different dilutions against Fusarium oxysporum f.sp. lactucae, as compared to an untreated control inoculated with Fusarium oxysporum f.sp. lactucae.

FIG. 16C shows photographs of cell culture plates of Wild Type Strain 7084 and genetically modified strain 7084-G77 (5 dpi) at different dilutions against Fusarium oxysporum f.sp. lactucae, as compared to an untreated control inoculated with Fusarium oxysporum f sp. lactucae.

FIG. 17A shows the antifungal activity of Whole Cell Broths (WCB) obtained from Wild Type Strain 7084 and genetically modified strain 7084-G77 at different dilutions against Penicillium digitatum.

FIG. 17B shows photographs of cell culture plates of Wild Type Strain 7084 and genetically modified strain 7084-G77 (3 dpi) at different dilutions against Penicillium digitatum, as compared to an untreated control inoculated with Penicillium digitatum.

FIG. 18A shows the antifungal activity of Whole Cell Broths (WCB) obtained from Wild Type Strain 7084 and genetically modified strain 7084-G77 at different dilutions against Botrytis cinerea.

FIG. 18B shows photographs of cell culture plates of Wild Type Strain 7084 and genetically modified strain 7084-G77 (3 dpi) at 1:100 dilutions against Botrytis cinerea, as compared to an untreated control inoculated with Botrytis cinerea.

FIG. 19 shows the antibacterial activity of Whole Cell Broths (WCB) obtained from Wild Type Strain 7084 and genetically modified strains 7084-G77 and 7084-G95 at different dilutions against Agrobacterium tumefaciens.

FIG. 20A is a culture photograph of the primary colony morphology of Wild Type strain 7084, showing smooth, rounded colonies. Most 7084 WT colonies were of this morphology.

FIG. 20B is a culture photograph of the alternate colony morphology of Wild Type strain 7084, showing serrated-edged colonies. A minority of 7084 WT colonies were of this morphology. One of the edited strains, 7084-G67, only exhibited this alternative morphology and did not exhibit any smooth, rounded colonies.

FIG. 21 shows the percent bioavailability of different secondary metabolites for the wild type and edited strains of 7084.

FIG. 22A is a photograph of a colony of wild type strain 14416.

FIG. 22B is a photograph of a colony of 14416-G2.

FIG. 23 is a graph of cyclic lipopeptide production of Bacillus velezensis strains 7084 and 14416, and their genome-edited (kinA knockout) mutants G77 and G2, respectively.

FIG. 24 is a graph of polyketide production of Bacillus velezensis strains 7084 and 14416, and their genome-edited (kinA knockout) mutants 7084-G77 and 14416-G2, respectively.

FIG. 25 shows the percent bioavailability of different secondary metabolites for the wild type and edited strains of 7084 and 14416.

FIG. 26 shows the Total Viable Cell (TVC) counts and Spore counts wild type and edited strains of 7084 and 14416.

FIG. 27A shows the Fusarium graminearum fungal inhibition capabilities of wild type and kinA knockout strains of 14416.

FIG. 27B are photographs of the Fusarium graminearum fungal inhibition assays of wild type and kinA knockout strains of 14416 at 3 days after inoculation.

FIG. 27C are photographs of the Fusarium graminearum fungal inhibition assays of wild type and kinA knockout strains of 14416 at 9 days after inoculation.

FIG. 28A shows the Fusarium oxysporum fungal inhibition capabilities of wild type and kinA knockout strains of 14416.

FIG. 28B are photographs of the Fusarium oxysporum fungal inhibition assays of wild type and kinA knockout strains of 14416 at 3 days after inoculation.

FIG. 28C are photographs of the Fusarium oxysporum fungal inhibition assays of wild type and kinA knockout strains of 14416 at 9 days after inoculation.

FIG. 29 shows the Pythium ultimum fungal inhibition capabilities of wild type and kinA knockout strains of 14416.

FIG. 30A shows the Fusarium graminearum fungal inhibition capabilities of wild type and kinA knockout strains of 102504.

FIG. 30B are photographs of the Fusarium graminearum fungal inhibition assays of wild type and kinA knockout strains of 102504.

FIG. 31A shows the Fusarium oxysporum fungal inhibition capabilities of wild type and kinA knockout strains of 102504.

FIG. 31B are photographs of the Fusarium oxysporum fungal inhibition assays of wild type and kinA knockout strains of 102504.

FIG. 32A shows the Penicillium expansum fungal inhibition capabilities of wild type and kinA knockout strains of 102504.

FIG. 32B are photographs of the Penicillium expansum fungal inhibition assays of wild type and kinA knockout strains of 102504.

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

SEQ ID NO:1 is the Strain 7084 16S DNA sequence from Bacillus velezensis.

SEQ ID NO:2 is the Strain 6011 16S DNA sequence from Bacillus licheniformis.

SEQ ID NO:3 is the Strain 39400 16S DNA sequence from Bacillus thuringiensis.

SEQ ID NO:4 is the Strain 102504 16S DNA sequence from Paenibacillus polymyxa.

SEQ ID NO:5 is the Strain 101545 16S DNA sequence from Paenibacillus peoriae.

SEQ ID NO:6 is the Strain 14416 16S DNA sequence from Bacillus velezensis.

SEQ ID NO:7 is the Strain 65935 16S DNA sequence from Bacillus pumilus.

SEQ ID NO:8 is the Strain 7084 kinA gene DNA sequence from Bacillus velezensis.

SEQ ID NO:9 is the Strain 102504 kinA gene DNA sequence from Paenibacillus polymyxa.

SEQ ID NO:10 is the Strain 14416 kinA gene DNA sequence from Bacillus velezensis.

In some cases, the microorganisms described in this Application were deposited with the Agricultural Research Service Culture Collection (NRRL), which is an International Depositary Authority, located at 1815 North University Street, Peoria, IL 61604, USA. The deposits were made under the terms of the Budapest Treaty on the International Recognition of the Deposit of Microorganisms for the Purposes of Patent Procedure. The deposits were made in accordance with, and to satisfy, the criteria set forth in 37 C.F.R. §§ 1.801-1.809 and the Manual of Patent Examining Procedure §§ 2402-2411.05.

Strain 7084 (Bacillus velezensis) was deposited with the NRRL on 3 Jul. 2019 as NRRL Deposit No. B-67810.

Strain 39400 (Bacillus thuringiensis) was deposited with the NRRL on 26 Jan. 2022 as NRRL Deposit No. B-68090.

### DETAILED DESCRIPTION

Biological bactericides and fungicides control bacterial and fungal disease through the production of secondary metabolites, including but not limited to cyclic lipopeptides (CLPs) and/or polyketides. Wild type (naturally-occurring) bacteria can be good producers of cyclic lipopeptides, but production of these chemistries can only be optimized so much through non-targeted strain improvement and fermentation optimization to achieve produce the amount of secondary metabolites required to inhibit bacterial and fungal disease. In order to achieve much higher levels of CLP production, bacterial strains need to be engineered. By creating engineered bacteria that produce more secondary metabolites, a biologically-based disease control product can be created that has better performance, cheaper costs to produce, and lower use rates than non-engineered biologically-based disease control products.

Disclosed herein are methods for creating and identifying microorganisms that produce metabolites that are useful in agriculture and other fields.

In some embodiments, the compositions are metabolites. In some embodiments, the compositions or metabolites are lipopeptides. In some embodiments, the compositions include isolated and purified lipopeptide metabolites. In some embodiments, the compositions include supernatant compositions including one or more lipopeptide(s). In some embodiments, the compositions are exposed to non-biological temperatures, for example autoclaving.

The final engineered bacterial strain produces significantly more CLPs, for example increased production per cell, than the un-engineered parent bacterial strain or other known biological disease controlling strains. Fermentation strategies include typical batch and fed-batch processes, as well as emulsion microreactor fermentations and artificially precipitating CLPs during fermentation to minimize the effective concentration in the culture liquid. Formulation strategies could include increasing CLP solubility, encapsulation, and combination with metabolites from other microbes. The final product can be applied at a lower rate with equivalent or better performance than the un-engineered parent bacteria as well as other known biological disease controlling strains. The specific engineering in the product was determined by the best performing bacteria.

In some aspects, increases in production of polyketides, which have antibacterial properties, were achieved through genome editing of strains.

Following submerged or solid-state fermentation of the high CLP-producing engineered derivative, a version of the fungicidal product could be developed which is completely devoid of living microbial cells. The downstream processing used to accomplish this could include a method, or combination of methods, such as centrifugation, filtration, fermentation broth clarification, heat treatment, autoclaving, and/or chemical treatment of the whole broth which kills the bacterial cells. During the process of removing bacterial cells, the final product may or may not be concentrated.

In some embodiments, the lipopeptide metabolites are useful for the biocontrol of phytopathogens or pests, for example nematodes. In some embodiments, the phytopathogens are oomycetes.

Additionally disclosed herein are compositions including metabolites that impart a beneficial property to a plant, or the microorganisms that produce the metabolites.

Also disclosed herein are methods of using the compositions in agriculture and other fields.

While the following terms are believed to be well understood by one of ordinary skill in the art, the following are set forth to facilitate explanation of the presently disclosed subject matter.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “about” refers to up to ±10% of the recited value. For example, the term “about” can refer to ±1%, ±2%, ±3%, ±4%, ±5%, ±6%, ±7%, ±8%, ±9%, ±10%, of the recited value, or non-integer percentages thereof. By way of additional example, the term “about” may refer to ±0.2 minutes with respect to the retention times recited herein.

As used herein the terms “microorganism” or “microbe” should be taken broadly. These terms are used interchangeably and include, but are not limited to, the two prokaryotic domains, Bacteria and Archaea, as well as eukaryotic Fungi and Protists.

As used herein the terms “microorganism” or “microbe” should be taken broadly. These terms are used interchangeably and include, but are not limited to, the two prokaryotic domains, Bacteria and Archaea, as well as eukaryotic Fungi and Protists. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms) or more than one cell (e.g., multi-cellular microorganism). A “population of microorganisms” may thus refer to multiple cells of a single microorganism, in which the cells share common genetic derivation.

As used herein, the term “bacterium” or “bacteria” refers in general to any prokaryotic organism, and may reference an organism from either Kingdom Eubacteria (Bacteria), Kingdom Archaebacteria (Archae), or both. In some cases, bacterial genera or other taxonomic classifications have been reassigned due to various reasons (such as but not limited to the evolving field of whole genome sequencing), and it is understood that such nomenclature reassignments are within the scope of any claimed taxonomy. For example, certain species of the genus Erwinia have been described in the literature as belonging to genus Pantoea (Zhang, Y., Qiu, S. Examining phylogenetic relationships of Erwinia and Pantoea species using whole genome sequence data. Antonie van Leeuwenhoek 108, 1037-1046 (2015).).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

When referring to polynucleotide or a genome, a “locus” refers to a specific polynucleotide sequence that can be accurately and consistently identified. For example, a particular locus in a genome may be identified based on sequence similarity or percent identity to a known gene. In another example, a particular locus within a polynucleotide sequence may be identified based on the composition of certain motifs.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence. The term “artificial” may be considered synonymous to “synthetic” in this context.

As used herein, the term “homologous” or “homologue”, “homolog”, or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

As used herein, the term “nucleotide change” refers to, e.g., nucleotide substitution, deletion, insertion, chemical alteration, or any of the preceding, as is well understood in the art.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g., at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g., Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

The term “16S” refers to the DNA sequence of the 16S ribosomal RNA (rRNA) sequence of a bacterium. 16S rRNA gene sequencing is a well-established method for studying phylogeny and taxonomy of bacteria. [00166] As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, J. W. One Fungus=One Name: DNA and fungal nomenclature twenty years after PCR. IMA Fungus 2, 113-120 (2011).). With the development of genomic sequencing, it became evident that taxonomic classification based on molecular phylogenetics did not align with morphological-based nomenclature (Shenoy, B. D., Jeewon, R. and Hyde, K. D. (2007). Impact of DNA sequence-data on the taxonomy of anamorphic fungi. Fungal Diversity 26: 1-54.). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, D. L. Managing and coping with names of pleomorphic fungi in a period of transition. IMA Fungus 3, 15-24 (2012)).

The term “Internal Transcribed Spacer” (“ITS”) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (“LSU”) sequence is used to identify fungi. LSU gene sequencing is a well-established method for studying phylogeny and taxonomy of fungi. Some fungal microbes of the present invention may be described by an ITS sequence and some may be described by an LSU sequence. Both are understood to be equally descriptive and accurate for determining taxonomy.

The term “microbial community” means a group of microbes comprising two or more genera, and/or species, and/or or strains. Unlike microbial consortia, a microbial community does not have to be carrying out a common function, or does not have to be participating in, or leading to, or correlating with, a recognizable parameter or plant phenotypic trait. The community may comprise one or more species, or strains of a species, of microbes. In some instances, the microbes coexist within the community symbiotically.

The term “microbial consortia” or “microbial consortium” refers to a subset of a microbial community of individual microbes, which can be described as carrying out a common function, or can be described as participating in, or leading to, or correlating with, a recognizable parameter or plant phenotypic trait. Consortia of microbes identified herein can each provide different aspects of a desired outcome (e.g., plant biotic stress control), and/or can work with one another in an additive fashion (e.g., one microbe providing control of one biotic stressor and another microbe providing control of a different biotic stressor), and/or can work with each other in a synergistic fashion (e.g., two or more microbes providing a level of biotic stress control to a plant greater than the sum of any individual microbe's effect).

The term “accelerated microbial selection” or “AMS” is used interchangeably with the term “directed microbial selection” or “DMS” and refers to the iterative selection methodology that was utilized, in some embodiments of the disclosure, to derive the claimed microbial species or consortia of said species.

As used herein, “isolate,” “isolated,” “isolated microbe,” and like terms, are intended to mean that the one or more microorganisms has been separated from at least one of the materials with which it is associated in a particular environment (for example soil, water, plant tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an agricultural carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g., In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a plant. By way of example, the media may be natural or artificial including, but not limited to: soil, potting mixes, bark, vermiculite, hydroponic solutions alone and applied to solid plant support systems, and tissue culture gels. It should be appreciated that the media may be used alone or in combination with one or more other media. It may also be used with or without the addition of exogenous nutrients and physical support systems for roots and foliage.

In one embodiment, the growth medium is a naturally occurring medium such as soil, sand, mud, clay, humus, regolith, rock, or water. In another embodiment, the growth medium is artificial. Such an artificial growth medium may be constructed to mimic the conditions of a naturally occurring medium; however, this is not necessary. Artificial growth media can be made from one or more of any number and combination of materials including sand, minerals, glass, rock, water, metals, salts, nutrients, water. In one embodiment, the growth medium is sterile. In another embodiment, the growth medium is not sterile.

The medium may be amended or enriched with additional compounds or components, for example, a component which may assist in the interaction and/or selection of specific groups of microorganisms with the plant and each other. For example, antibiotics (such as penicillin) or sterilants (for example, quaternary ammonium salts and oxidizing agents) could be present and/or the physical conditions (such as salinity, plant nutrients (for example organic and inorganic minerals (such as phosphorus, nitrogenous salts, ammonia, potassium and micronutrients such as cobalt and magnesium), pH, and/or temperature) could be amended.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant part” is synonymous to a “portion” of a plant, and refers to any part of the plant, and can include distinct tissues and/or organs, and may be used interchangeably with the term “tissue” throughout.

“Progeny” comprises any subsequent generation of an organism, produced via sexual or asexual reproduction.

As used herein, the term “plant element” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, the term “cultivar” refers to a variety, strain, or race, of plant that has been produced by horticultural or agronomic techniques and is not normally found in wild populations.

As used herein, the term “molecular marker”, “marker”, or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed by the average person skilled in molecular-biological techniques.

As used herein, the term “trait” refers to a characteristic or phenotype. For example, in the context of some embodiments of the present disclosure, yield of a crop relates to the amount of marketable biomass produced by a plant (e.g., fruit, fiber, grain). Desirable traits may also include other plant characteristics, including but not limited to: water use efficiency, nutrient use efficiency, production, mechanical harvestability, fruit maturity, shelf life, pest/disease resistance, early plant maturity, tolerance to stresses, etc. A trait may be inherited in a dominant or recessive manner, or in a partial or incomplete-dominant manner. A trait may be monogenic (i.e., determined by a single locus) or polygenic (i.e., determined by more than one locus) or may also result from the interaction of one or more genes with the environment.

As used herein, the term “phenotype” refers to the observable characteristics of an individual cell, cell culture, organism (e.g., a plant), or group of organisms which results from the interaction between that individual's genetic makeup (i.e., genotype) and the environment.

As used herein, “improved” should be taken broadly to encompass improvement of a characteristic of a plant, as compared to a control plant, or as compared to a known average quantity associated with the characteristic in question. For example, “improved” plant biomass associated with application of a beneficial microbe, or consortia, of the disclosure can be demonstrated by comparing the biomass of a plant treated by the microbes taught herein to the biomass of a control plant not treated. Alternatively, one could compare the biomass of a plant treated by the microbes taught herein to the average biomass normally attained by the given plant, as represented in scientific or agricultural publications known to those of skill in the art. In the present disclosure, “improved” does not necessarily demand that the data be statistically significant (e.g., p<0.05); rather, any quantifiable difference demonstrating that one value (e.g., the average treatment value) is different from another (e.g., the average control value) can rise to the level of “improved.”

As used herein, “inhibiting and suppressing” and like terms should not be construed to require complete inhibition or suppression, although this may be desired in some embodiments.

The terms “inhibiting”, “suppressing”, “tolerance”, “resistance” and like terms can broadly be categorized as “control”. For example, if a mutation of a gene in an organism may render it capable of conferring tolerance to a particular composition that would otherwise cause a deleterious effect, said mutation can be considered to render the organism capable of providing control over that composition.

As used herein, the term “genotype” refers to the genetic makeup of an individual cell, cell culture, tissue, organism (e.g., a plant), or group of organisms.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein

“Agronomic trait potential” is intended to mean a capability of a plant element for exhibiting a phenotype, preferably an improved agronomic trait, at some point during its life cycle, or conveying said phenotype to another plant element with which it is associated in the same plant.

In some embodiments, the cell or organism has at least one heterologous trait. As used herein, the term “heterologous trait” refers to a phenotype imparted to a cell or organism by an exogenous molecule or other organism (e.g., a microbe), DNA segment, heterologous polynucleotide or heterologous nucleic acid.

Various changes in phenotype are of interest to the present disclosure, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present disclosure

A “synthetic combination” can include a combination of a plant and a microbe, or a plant and a composition, of the disclosure. The combination may be achieved, for example, by coating the surface of a seed of a plant, such as an agricultural plant, or host plant tissue (root, stem, leaf, etc.), with a microbe of the disclosure. Further, a “synthetic combination” can include a combination of microbes of various strains or species. Synthetic combinations have at least one variable that distinguishes the combination from any combination that occurs in nature. That variable may be, inter alia, a concentration of microbe on a seed or plant tissue that does not occur naturally, or a combination of microbe and plant that does not naturally occur, or a combination of microbes or strains that do not occur naturally together. In each of these instances, the synthetic combination demonstrates the hand of man and possesses structural and/or functional attributes that are not present when the individual elements of the combination are considered in isolation.

In some embodiments, a microbe can be “endogenous” to a seed or plant. As used herein, a microbe is considered “endogenous” to a plant or seed, if the microbe is derived from the plant specimen from which it is sourced. That is, if the microbe is naturally found associated with said plant. In embodiments in which an endogenous microbe is applied to a plant, then the endogenous microbe is applied in an amount that differs from the levels found on the plant in nature. Thus, a microbe that is endogenous to a given plant can still form a synthetic combination with the plant, if the microbe is present on said plant at a level that does not occur naturally.

In some embodiments, a composition (such as a microbe) can be “heterologous” (also termed “exogenous”) to another composition (such as a seed or plant), and in some aspects is referred to herein as a “heterologous composition”. As used herein, a microbe is considered “heterologous” to a plant or seed, if the microbe is not derived from the plant specimen from which it is sourced. That is, if the microbe is not naturally found associated with said plant. For example, a microbe that is normally associated with leaf tissue of a maize plant is considered exogenous to a leaf tissue of another maize plant that naturally lacks said microbe. In another example, a microbe that is normally associated with a maize plant is considered exogenous to a wheat plant that naturally lacks said microbe. In general, “heterologous” refers to a state conferred by the non-naturally-occurring association of one composition (e.g., chemical, molecule, seed, plant, gene) with another of the same or different type. Such a non-naturally-occurring combination may further be referred to as a “synthetic combination”.

A composition is “heterologously disposed” when mechanically or manually applied, artificially inoculated, associated with, or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment (for example, drought). In some embodiments, such a manner is contemplated to be selected from the group consisting of: the presence of the microbe; presence of the microbe in a different number of cells, concentration, or amount; the presence of the microbe in a different plant element, tissue, cell type, or other physical location in or on the plant; the presence of the microbe at different time period, e.g., developmental phase of the plant or plant element, time of day, time of season, and combinations thereof. In some embodiments, “heterologously disposed” means that the microbe being applied to a different tissue or cell type of the plant element than that in which the microbe is naturally found. In some embodiments, “heterologously disposed” means that the microbe is applied to a developmental stage of the plant element, seedling, or plant in which said microbe is not naturally associated, but may be associated at other stages. For example, if a microbe is normally found at the flowering stage of a plant and no other stage, a microbe applied at the seedling stage may be considered to be heterologously disposed. In some embodiments, a microbe is heterologously disposed the microbe is normally found in the root tissue of a plant element but not in the leaf tissue, and the microbe is applied to the leaf. In another non-limiting example, if a microbe is naturally found in the mesophyll layer of leaf tissue but is being applied to the epithelial layer, the microbe would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the microbe being applied is at a greater concentration, number, or amount of the plant element, seedling, or plant, than that which is naturally found in said plant element, seedling, or plant. For example, a microbe is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said microbe. In another non-limiting example, a microbe that is naturally found in a tissue of a cupressaceous tree would be considered heterologous to tissue of a maize, wheat, cotton, soybean plant. In another example, a microbe that is naturally found in leaf tissue of a maize, spring wheat, cotton, soybean plant is considered heterologous to a leaf tissue of another maize, spring wheat, cotton, soybean plant that naturally lacks said microbe, or comprises the microbe in a different quantity.

Microbes can also be “heterologously disposed” on a given plant tissue. This means that the microbe is placed upon a plant tissue that it is not naturally found upon. For instance, if a given microbe only naturally occurs on the roots of a given plant, then that microbe could be exogenously applied to the above-ground tissue of a plant and would thereby be “heterologously disposed” upon said plant tissue. As such, a microbe is deemed heterologously disposed, when applied on a plant that does not naturally have the microbe present or does not naturally have the microbe present in the number that is being applied.

The compositions and methods herein may provide for a “modulated” “agronomic trait” or “trait of agronomic importance” to a host plant, which may include, but not be limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation. By the term “modulated”, it is intended to refer to a change in a characteristic, such as an agronomic trait, that is changed by virtue of the presence of the microbe(s), exudate, broth, metabolite, etc. In aspects, the modulation provides for the imparting of a trait, such as a trait of agronomic importance.

### Microbes and Microorganisms

As used herein the term “microorganism” should be taken broadly. It includes, but is not limited to, prokaryotic Bacteria and Archaea, as well as eukaryotic Fungi and Protists.

By way of example, the microorganisms may include: Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium, Variovorax and Halomonas), Firmicutes (such as Bacillus, Paenibacillus, Lactobacillus, Mycoplasma, and Acetobacterium), Actinobacteria (such as Brevibacterium, Janibacter, Streptomyces, Rhodococcus, Microbacterium, Curtobacterium, Cellulomonas, and Nocardioides), and the fungi Ascomycota (such as Trichoderma, Ampelomyces, Coniothyrium, Paecoelomyces, Penicillium, Cladosporium, Hypocrea, Beauveria, Metarhizium, Verticullium, Cordyceps, Pichea, and Candida), Basidiomycota (such as Coprinus, Corticium, and Agaricus) and Oomycota (such as Pythium), and Mucoromycota (such as Mucor, and Mortierella); as well as Orbilia/Arthrobotrys, Lysinibacillus, Microbacterium, Talaromyces, Arthrobacter, Kosakonia, Masillia, Novosphingobium, and Tumebacillus.

In a particular embodiment, the microorganism is an endophyte, or an epiphyte, or a microorganism inhabiting the plant rhizosphere, rhizoplane, or rhizosheath. That is, the microorganism may be found present in the soil material adhered to the roots of a plant or in the area immediately adjacent a plant's roots.

In one embodiment, the microorganism is an endophyte. Endophytes may benefit host plants by preventing pathogenic organisms from colonizing them. Extensive colonization of the plant tissue by endophytes creates a “barrier effect,” where the local endophytes outcompete and prevent pathogenic organisms from taking hold. Endophytes may also produce chemicals which inhibit the growth of competitors, including pathogenic organisms.

In certain embodiments, the microorganism is unculturable. This should be taken to mean that the microorganism is not known to be culturable or is difficult to culture using methods known to one skilled in the art.

Microorganisms of the present disclosure may be collected or obtained from any source or contained within and/or associated with material collected from any source.

In an embodiment, the microorganisms are obtained from any general terrestrial environment, including its soils, plants, fungi, animals (including invertebrates) and other biota, including the sediments, water and biota of lakes and rivers; from the marine environment, its biota and sediments (for example sea water, marine muds, marine plants, marine invertebrates (for example sponges), marine vertebrates (for example, fish)); the terrestrial and marine geosphere (regolith and rock, for example crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, road surfaces).

In another embodiment the microorganisms are collected from a source likely to favor the selection of appropriate microorganisms. By way of example, the source may be a particular environment in which it is desirable for other plants to grow, or which is thought to be associated with terroir. In another example, the source may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the microorganisms may be collected from commercial crops grown in such environments, or more specifically from individual crop plants best displaying a trait of interest amongst a crop grown in any specific environment, for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste, or smell. The microorganisms may be collected from a plant of interest or any material occurring in the environment of interest, including fungi and other animal and plant biota, soil, water, sediments, and other elements of the environment as referred to previously. In certain embodiments, the microorganisms are individual isolates separated from different environments.

In one embodiment, a microorganism or a combination of microorganisms, of use in the methods of the disclosure may be selected from a pre-existing collection of individual microbial species or strains based on some knowledge of their likely or predicted benefit to a plant. For example, the microorganism may be predicted to: improve nitrogen fixation; release phosphate from the soil organic matter; release phosphate from the inorganic forms of phosphate (e.g., rock phosphate); “fix carbon” in the root microsphere; live in the rhizosphere of the plant thereby assisting the plant in absorbing nutrients from the surrounding soil and then providing these more readily to the plant; increase the number of nodules on the plant roots and thereby increase the number of symbiotic nitrogen fixing bacteria (e.g., Rhizobium species) per plant and the amount of nitrogen fixed by the plant; elicit plant defensive responses such as ISR (induced systemic resistance) or SAR (systemic acquired resistance) which help the plant resist the invasion and spread of pathogenic microorganisms; compete with microorganisms deleterious to plant growth or health by antagonism, or competitive utilization of resources such as nutrients or space; change the color of one or more part of the plant, or change the chemical profile of the plant, its smell, taste or one or more other quality.

In one embodiment a microorganism or combination of microorganisms is selected from a pre-existing collection of individual microbial species or strains that provides no knowledge of their likely or predicted benefit to a plant. For example, a collection of unidentified microorganisms isolated from plant tissues without any knowledge of their ability to improve plant growth or health, or a collection of microorganisms collected to explore their potential for producing compounds that could lead to the development of pharmaceutical drugs.

In one embodiment, the microorganisms are acquired from the source material (for example, soil, rock, water, air, dust, plant or other organism) with or within which they naturally reside. The microorganisms may be provided in any appropriate form, having regard to its intended use in the methods of the disclosure. However, by way of example only, the microorganisms may be provided as an aqueous suspension, gel, homogenate, granule, powder, slurry, live organism, or dried material.

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either applied directly to the plant growth medium, or concentrated by filtering or centrifugation, diluted to an appropriate concentration and applied to the plant growth medium with the bulk of the salt removed. By way of further example, microorganisms from mineralized or toxic sources may be similarly treated to recover the microbes for application to the plant growth material to minimize the potential for damage to the plant.

In another embodiment, the microorganisms are used in a crude form, in which they are not isolated from the source material in which they naturally reside. For example, the microorganisms are provided in combination with the source material in which they reside; for example, as soil, or the roots, seed, or foliage of a plant. In this embodiment, the source material may include one or more species of microorganisms.

In some embodiments, a mixed population of microorganisms is used in the methods of the disclosure.

In embodiments of the disclosure where the microorganisms are isolated from a source material (for example, the material in which they naturally reside), any one or a combination of a number of standard techniques which will be readily known to skilled persons may be used. However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

Whilst not essential, in one embodiment, the material containing the microorganisms may be pre-treated prior to the isolation process in order to either multiply all microorganisms in the material, or select portions of the microbial population, either by enriching the material with microbial nutrients (for example, by pasteurizing the sample to select for microorganisms resistant to heat exposure (for example, bacilli), or by exposing the sample to low concentrations of an organic solvent or sterilant (for example, household bleach) to enhance the survival of spore-forming or solvent-resistant microorganisms). Microorganisms can then be isolated from the enriched materials or materials treated for selective survival, as above.

In an embodiment of the disclosure, endophytic or epiphytic microorganisms are isolated from plant material. Any number of standard techniques known in the art may be used and the microorganisms may be isolated from any appropriate tissue in the plant, including for example root, stem and leaves, and plant reproductive tissues. By way of example, conventional methods for isolation from plants typically include the sterile excision of the plant material of interest (e.g., root or stem lengths, leaves), surface sterilization with an appropriate solution (e.g., 2% sodium hypochlorite), after which the plant material is placed on nutrient medium for microbial growth (See, for example, Strobel G and Daisy B (2003) Microbiology and Molecular Biology Reviews 67 (4): 491-502; Zinniel D K et al. (2002) Applied and Environmental Microbiology 68 (5): 2198-2208).

In one embodiment of the disclosure, the microorganisms are isolated from root tissue. Further methodology for isolating microorganisms from plant material are detailed hereinafter.

In one embodiment, the microbial population is exposed (prior to the method or at any stage of the method) to a selective pressure. For example, exposure of the microorganisms to pasteurization before their addition to a plant growth medium (preferably sterile) is likely to enhance the probability that the plants selected for a desired trait will be associated with spore-forming microbes that can more easily survive in adverse conditions, in commercial storage, or if applied to seed as a coating, in an adverse environment.

In certain embodiments, as mentioned herein before, the microorganism(s) may be used in crude form and need not be isolated from a plant or a media. For example, plant material or growth media which includes the microorganisms identified to be of benefit to a selected plant may be obtained and used as a crude source of microorganisms for the next round of the method or as a crude source of microorganisms at the conclusion of the method. For example, whole plant material could be obtained and optionally processed, such as mulched or crushed. Alternatively, individual tissues or parts of selected plants (such as leaves, stems, roots, and seeds) may be separated from the plant and optionally processed, such as mulched or crushed. In certain embodiments, one or more part of a plant which is associated with the second set of one or more microorganisms may be removed from one or more selected plants and, where any successive repeat of the method is to be conducted, grafted on to one or more plant used in any step of the plant breeding methods.

### Sourcing of Microbes

The microbes of the present disclosure were obtained, among other places, at various locales in New Zealand and the United States

### Isolation and Culturing of Microbes

Microbes were identified by utilizing standard microscopic techniques to characterize the microbes' phenotype, which was then utilized to identify the microbe to a taxonomically recognized species.

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for isolated bacteria of the disclosure, biologically pure isolates can be obtained through repeated subculture of biological samples, each subculture followed by streaking onto solid media to obtain individual colonies. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze-dried liquid formulations and cultures stored long term at −70° C. in solutions containing glycerol are contemplated for use in providing formulations of the present inventions.

The bacteria of the disclosure can be propagated in a “culture medium”, which may comprise a liquid medium or solid medium, under aerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the bacterial strains include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the bacterial strains, essentially, between 20° C. and 46° C. In some aspects, a temperature range is 15° C.-40° C. For optimal growth, in some embodiments, the medium can be adjusted to pH 7.0-7.4. It will be appreciated that commercially available media may also be used to culture the bacterial strains, such as Nutrient Broth or Nutrient Agar available from Difco, Detroit, MI. It will be appreciated that cultivation time may differ depending on the type of culture medium used and the concentration of sugar as a major carbon source.

In aspects, cultivation lasts between 24-96 hours. Bacterial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. It will be appreciated that the microbial strains may be cultured together when compatible culture conditions can be employed.

### Identification of Microbes

Microbes can be distinguished into a genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTm (the difference in the melting temperature between homologous and heterologous hybrids), under standard conditions, are considered to be members of the same species. Thus, populations that share greater than the aforementioned 70% threshold can be considered to be variants of the same species.

For bacterial microbes, the 16S rRNA sequences are often used for determining taxonomy and making distinctions between species, in that if a 16S rRNA sequence shares less than a specified % sequence identity from a reference sequence, then the two organisms from which the sequences were obtained are said to be of different species.

Thus, one could consider microbes to be of the same species, if they share at least 80%, 85%, 90%, 95%, 97%, 98%, or 99% sequence identity across the 16S or 16S rRNA or rDNA sequence. In some aspects, a microbe could be considered to be the same species only if it shares at least 95% identity.

Further, one could define microbial strains of a species, as those that share at least 80%, 85%, 90%, 95%, 97%, 98%, or 99% sequence identity across the 16S rRNA sequence.

Comparisons may also be made with 23S rRNA sequences against reference sequences. In some aspects, a microbe could be considered to be the same strain only if it shares at least 95% identity. In some embodiments, “substantially similar genetic characteristics” means a microbe sharing at least 95% identity.

For fungal microbes, the ITS (Internal Transcriber Sequence) is often used for identification of taxonomy. Among the regions of the ribosomal cistron, the internal transcribed spacer (ITS) region has the highest probability of successful identification for the broadest range of fungi, with the most clearly defined barcode gap between inter- and intraspecific variation, and has been proposed as the formal fungal identification sequence (Schoch et al., PNAS Apr. 17, 2012 109 (16) 6241-6246).

In one embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity SEQ ID NO:1.

In one embodiment, microbes of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity SEQ ID NO:1.

In one embodiment, microbial consortia of the present disclosure include two or more microbes that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity SEQ ID NO:1.

In one embodiment, microbial consortia of the present disclosure include two or more microbial strains, wherein at least one of those comprises a polynucleotide sequence that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity SEQ ID NO:1.

In one embodiment, microbial consortia of the present disclosure include two or more microbial strains, wherein at least one of those comprises a polynucleotide sequence that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity SEQ ID NO:1.

Unculturable microbes often cannot be assigned to a definite species in the absence of a phenotype determination, the microbes can be given a candidatus designation within a genus provided their 16S rRNA sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics are made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity and atmospheric conditions, growth in presence of different sole carbon and nitrogen sources. One of ordinary skill in the art would be reasonably apprised as to the phenotypic traits that define the genera of the present disclosure. For instance, colony color, form, and texture on a particular agar (e.g., YMA) can be used to identify species of Rhizobium.

In one embodiment, bacterial microbes taught herein were identified utilizing 16S rRNA gene sequences. It is known in the art that 16S rRNA contains hypervariable regions that can provide species/strain-specific signature sequences useful for bacterial identification. In the present disclosure, many of the microbes were identified via partial (500-1200 bp) 16S rRNA sequence signatures. In aspects, each strain represents a pure colony isolate that was selected from an agar plate. Selections were made to represent the diversity of organisms present based on any defining morphological characteristics of colonies on agar medium. The medium used, in embodiments, was R2A, PDA, Nitrogen-free semi-solid medium, or MRS agar. Colony descriptions of each of the ‘picked’ isolates were made after 24-hour growth and then entered into our database. Sequence data was subsequently obtained for each of the isolates.

Phylogenetic analysis using the 16S rRNA gene was used to define “substantially similar” species belonging to common genera and also to define “substantially similar” strains of a given taxonomic species. Further, we recorded physiological and/or biochemical properties of the isolates that can be utilized to highlight both minor and significant differences between strains that could lead to advantageous behavior on plants.

### Microbial Consortia

In aspects, the disclosure provides microbial consortia comprising a combination of at least any two microbes.

In certain embodiments, the consortia of the present disclosure comprise two microbes, or three microbes, or four microbes, or five microbes, or six microbes, or seven microbes, or eight microbes, or nine microbes, or ten or more microbes. Said microbes of the consortia are different microbial species, or different strains of a microbial species.

In some embodiments, the disclosure provides consortia, comprising at least one isolated microbial species and/or strains.

### Improvement of Traits in Plants

The present disclosure utilizes microbes to impart beneficial properties (or beneficial traits) to desirable plant species, such as agronomic species of interest. In the current disclosure, the terminology “beneficial property” or “beneficial trait” is used interchangeably and denotes that a desirable plant phenotypic or genetic property of interest is modulated, by the application of a microbe or microbial consortia as described herein. As aforementioned, in some aspects, it may very well be that a metabolite produced by a given microbe is ultimately responsible for modulating or imparting a beneficial trait to a given plant.

There are a vast number of beneficial traits that can be modulated by the application of microbes, and/or compositions produced therefrom, of the disclosure. For instance, the microbes may have the ability to impart one or more beneficial properties to a plant species, for example: increased growth, increased yield, increased nitrogen utilization efficiency, increased stress tolerance, increased drought tolerance, increased photosynthetic rate, enhanced water use efficiency, increased pathogen resistance, modifications to plant architecture that don't necessarily impact plant yield, but rather address plant functionality, causing the plant to increase production of a metabolite of interest, etc.

In aspects, the microbes and compositions taught herein provide a wide range of agricultural applications, including: improvements in yield of grain, fruit, and flowers, improvements in growth of plant parts, improved ability to utilize nutrients (e.g., nitrogen, phosphate, and the like), improved resistance to disease, biopesticidal effects including improved resistance to fungi and nematodes; improved survivability in extreme climate, and improvements in other desired plant phenotypic characteristics.

In some aspects, the isolated microbes, consortia, and/or compositions of the disclosure can be applied to a plant, in order to modulate or alter a plant characteristic such as altered oil content, altered protein content, altered seed carbohydrate composition, altered seed oil composition, altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved nutrient utilization (e.g., phosphate, potassium, and the like), improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, reduced pathogen levels (e.g., via the excretion of metabolites that impair pathogen survival), pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome relative to a reference plant.

In some aspects, the isolated microbes, consortia, and/or compositions of the disclosure can be applied to a plant, in order to modulate in a negative way, a particular plant characteristic. For example, in some aspects, the microbes of the disclosure are able to decrease a phenotypic trait of interest, as this functionality can be desirable in some applications. For instance, the microbes of the disclosure may possess the ability to decrease root growth or decrease root length. Or the microbes may possess the ability to decrease shoot growth or decrease the speed at which a plant grows, as these modulations of a plant trait could be desirable in certain applications.

Stress in plants refers to external conditions that adversely affect growth, development, or productivity of plants. Stresses trigger a wide range of plant responses like altered gene expression, cellular metabolism, changes in growth rates, crop yields, etc. A plant stress usually reflects some sudden changes in environmental condition. However, in stress tolerant plant species, exposure to a particular stress leads to acclimation to that specific stress in a time time-dependent manner. Plant stress can be divided into two primary categories namely abiotic stress and biotic stress. Abiotic stress imposed on plants by environment may be either physical or chemical, while as biotic stress exposed to the crop plants is a biological unit like diseases, insects, etc.

Biotic stress on a plant can be measured from parameters of either/both the plant or/and of the biotic stressor. Plant characteristics impacting health, vigor, and yield include aspects of canopy, roots, leaves, photosynthetic capability, stalks, stems, seed production, seed weight, fiber characteristics, and other measurable phenotypes. When the stressor is an insect or nematode, measurements of those organisms can include number, kind, developmental stage, health, nutritional status, percent live, etc. When the stressor is a phytopathogen, measurements can include identification of pathogen, biomass, area of infection, rate of growth, developmental state, nutritional status, reproductive status, etc.

In some embodiments, the isolated microbes, consortia, and/or compositions produced therefrom of the disclosure can be applied to a plant or plant element or growth medium, in order to impart biotic stress tolerance (e.g., reduce the presence and/or negative impact of insects, nematodes, and/or pathogens on the plant), abiotic stress tolerance (e.g., limitations of water, nutrients, light; cold or other extreme conditions), biostimulation, and/or post-harvest benefits to plants and/or plant parts. Suitably, in such embodiments, the microbes and/or compositions may be selected from the group consisting of Category 1, Category 2, and/or Category 3, as defined by criteria according to a method of any of the Examples.

The “positive biotic control potential”, or the ability of a microbe or composition produced therefrom to ameliorate the impact of a biotic stressor or improve the health of a target plant that is exposed to a biotic stressor, may be successfully predicted from the methods described herein. In some aspects, the biotic stressor is a nematode. In some aspects, the biotic stressor is a phytopathogen. In some aspects, the biotic stressor is a fungus.

A plant, plant tissue, plant part, or plant element treated with the compositions disclosed herein have improved tolerance to biotic stressors, such as phytopathogens or nematodes.

In some embodiments, the one or more beneficial traits are selected from promoting the colonization of the plant by one or more microorganisms, inhibiting the colonization of the plant by one or more microorganisms, promoting nutrient utilization in the plant, enhancing nutrient utilization efficiency in the plant, control of phytopathogens in the plant, and biocontrol of phytopathogens in the plant. In some embodiments, the one or more beneficial traits include promoting the colonization of the plant by one or more microorganisms. In some embodiments, the one or more beneficial traits include inhibiting the colonization of the plant by one or more microorganisms. In some embodiments, the one or more beneficial traits include promoting nutrient utilization in the plant. In some embodiments, the one or more beneficial traits include enhancing nutrient utilization efficiency in the plant. In some embodiments, the one or more beneficial traits include the control of phytopathogens in the plant. In some embodiments, the one or more beneficial traits include biocontrol of phytopathogens in the plant. In some embodiments, the one or more beneficial traits include biocontrol of phytopathogens in the plant, wherein the phytopathogens include one or more microorganisms of a genus selected from the group consisting of: Pythium, Penicillium, Phoma, Botrytis, Fusarium, Mucor, Colletotrichum, and Geotrichum. In some embodiments, the one or more beneficial traits include biocontrol of phytopathogens in the plant, wherein the phytopathogens include one or more microorganisms of a genus selected from the group consisting of: Pythium, Penicillium, Phoma, Botrytis, and Fusarium. In some embodiments, the one or more beneficial traits include biocontrol of phytopathogens in the plant, wherein the phytopathogens include one or more microorganisms of a genus selected from the group consisting of: Pythium, Penicillium, Phoma, and Fusarium. In some embodiments, the phytopathogen is of the genus Pythium. In some embodiments, the phytopathogen is of the genus Penicillium. In some embodiments, the phytopathogen is of the genus Phoma. In some embodiments, the phytopathogen is of the genus Botrytis. In some embodiments, the phytopathogen is of the genus Fusarium. In some embodiments, the phytopathogen is of the genus Mucor. In some embodiments, the phytopathogen is of the genus Colletotrichum. In some embodiments, the phytopathogen is of the genus Geotrichum. In some embodiments, the one or more beneficial traits include biocontrol of phytopathogens in the plant, wherein the phytopathogens include one or more microorganisms selected from the group consisting of: Pythium ultimum, Penicillium expansum, Penicillium digitatum, Botrytis cinerea, Fusarium oxysporum, Fusarium graminarum, Mucor circinelloides, Colletotrichum gloeosporoides, and Geotrichum candidum. In some embodiments, the one or more beneficial traits include biocontrol of phytopathogens in the plant, wherein the phytopathogens include one or more microorganisms selected from the group consisting of: Pythium ultimum, Penicillium expansum, Penicillium digitatum, Botrytis cinerea, and Fusarium oxysporum. In some embodiments, the one or more beneficial traits include biocontrol of phytopathogens in the plant, wherein the phytopathogens include one or more microorganisms selected from the group consisting of: Pythium ultimum, Penicillium expansum, Penicillium digitatum, and Fusarium oxysporum. In some embodiments, the phytopathogen is Pythium ultimum. In some embodiments, the phytopathogen is Penicillium expansum. In some embodiments, the phytopathogen is Penicillium digitatum. In some embodiments, the phytopathogen is Botrytis cinerea. In some embodiments, the phytopathogen is Fusarium oxysporum. In some embodiments, the phytopathogen is Fusarium graminarum. In some embodiments, the phytopathogen is Mucor circinelloides. In some embodiments, the phytopathogen is Colletotrichum gloeosporoides. In some embodiments, the phytopathogen is Geotrichum candidum. In some embodiments, the microorganism that produces metabolites that impart one or more beneficial traits to a plant belongs to a genus selected from the group consisting of: Bacillus, Pseudomonas, and Paenibacillus. In some embodiments, the microorganism is of the genus Bacillus. In some embodiments, the microorganism is of the genus Pseudomonas. In some embodiments, the microorganism is of the genus Paenibacillus.

In some embodiments, the microorganism that produces metabolites that impart one or more beneficial traits to a plant is obtained or derived from a Bacillus species. In some aspects, the microbe is selected from Bacillus licheniformis, Bacillus thuringiensis, Bacillus tequilensis, Bacillus pumilus, Bacillus amyloliquefaciens, Bacillus methylotrophicus, and Bacillus velezensis.

In some embodiments, the microorganism that produces metabolites that impart one or more beneficial traits to a plant is obtained or derived from a Paenibacillus species. In some aspects, the microbe is selected from Paenibacillus polymyxa and Paenibacillus peoriae.

Methods of Identifying Microorganisms that Produce Compositions that Impart Beneficial Traits

In one aspect, methods for identifying microorganisms that produce metabolites useful for a number of applications in agriculture or other fields are disclosed. For example, in some embodiments, methods for identifying microorganisms that produce metabolites that impart one or more beneficial traits to a plant are disclosed. In some embodiments, the methods identify microorganisms that produce metabolites useful for promoting the colonization of the plant by one or more microorganisms. In some embodiments, the methods identify microorganisms that produce metabolites useful for inhibiting the colonization of the plant by one or more microorganisms. In some embodiments, the methods identify microorganisms that produce metabolites useful for promoting nutrient utilization in the plant. In some embodiments, the methods identify microorganisms that produce metabolites useful for enhancing nutrient utilization efficiency in the plant. In some embodiments, the methods identify microorganisms that produce metabolites useful for biocontrol of phytopathogens in the plant. In some embodiments, the method for identifying microorganisms that produce metabolites that impart one or more beneficial traits to a plant includes: obtaining a first sample having one or more metabolites from a microorganism; obtaining a first metabolite profile from the first sample; and selecting the microorganism that produces one or more beneficial traits to a plant when the first metabolite profile has one or more unique elements, wherein at least one of the one or more unique elements corresponds to the one or more metabolites that impart the one or more beneficial traits to the plant.

As used herein, the term “unique” refers to a characteristic feature that is present in one item and absent in another item. By way of example, the present disclosure refers to a metabolite profile that has a “unique element”. In this context, the described metabolite profile possesses an element, signature, or feature that is absent from a reference metabolite profile to which the first metabolite profile is being compared. For example, if the metabolite profile is a chromatogram, a “unique element” may be a peak within the chromatogram that is absent from a reference chromatogram to which the first chromatogram is being compared.

In some embodiments, the microorganism may be cultured for 1 to 14 days in liquid media prior to obtaining the sample having one or more metabolites from the microorganism.

In some embodiments, the microorganism may be cultured for 1 to 14 days on solid media (e.g., agar) prior to obtaining the sample having one or more metabolites from the microorganism. In some embodiments, the microorganism may be cultured for 1 day, 2 days, 3 days, 4 days, 5 days, 6 days, 7 days, 8 days, 9 days, 10 days, 11 days, 12 days, 13 days, or 14 days prior to obtaining the sample having one or more metabolites. In some embodiments, the microorganism may be cultured for about 3 days to about 5 days prior to obtaining the sample having one or more metabolites. In some embodiments, the microorganism may be cultured for 4 days prior to obtaining the sample having one or more metabolites.

In some embodiments, the first sample having one or more metabolites from the microorganism that produces metabolites that impart one or more beneficial traits to a plant is selected from a supernatant sample of a culture that includes the microorganism, a whole broth sample of a culture that includes the microorganism, and an extract of a culture that includes the microorganism. In some embodiments, the first sample having one or more metabolites from the microorganism that produces metabolites that impart one or more beneficial traits to a plant is from the supernatant of a culture that includes the microorganism. The supernatant sample can be prepared by centrifuging a culture that includes the microorganism and separating the supernatant from pelleted cells and other solid components of the culture. Alternatively, the supernatant sample can be prepared by filtering the culture to separate the supernatant from the cells and other solid components of the culture. Those of ordinary skill in the art will appreciate that there are numerous other methods for isolating the supernatant from the culture and would therefore be compatible with the method disclosed herein. In some embodiments, the first sample having one or more metabolites from the microorganism that produces metabolites that impart one or more beneficial traits to a plant is a whole broth sample of a culture that includes the microorganism. In some embodiments, the first sample having one or more metabolites from the microorganism that produces metabolites that impart one or more beneficial traits to a plant is an extract of a culture that includes the microorganism. The extract can be prepared by lysing the cultured cells using techniques known to those of ordinary skill in the art and subsequently separating the soluble extracts from insoluble cell debris and other components of the culture, such as by centrifugation, for example.

In some embodiments, the sample having one or more metabolites from the microorganism includes one or more lipopeptides.

In some embodiments, obtaining a first metabolite profile includes subjecting the first sample having one or more metabolites to an analytical technique to identify the elements that comprise the first sample. The analytical technique may be any technique known to one of ordinary skill in the art that is capable of identifying the component metabolites within the first sample having one or more metabolites.

For example, the analytical technique may be, but is not limited to, a chemical separation, a chromatographic separation, nuclear magnetic resonance spectroscopy, mass spectrometry, or the like.

In some embodiments, obtaining the first metabolite profile includes subjecting the first sample having one or more metabolites to chromatographic separation.

In some embodiments, obtaining the first metabolite profile includes subjecting the first sample having one or more metabolites to chromatographic separation, wherein subjecting the first sample having one or more metabolites to chromatographic separation includes subjecting the first sample to a high-performance liquid chromatography (HPLC) method.

In some embodiments, the high-performance liquid chromatography method includes:
subjecting the sample to a column; and eluting the one or more metabolites with a gradient of a first and second mobile phase solvent. The “gradient of the first and second mobile phase solvent” refers to changing the composition of a mixture of the first and second mobile phase solvent over time. In some embodiments, the concentration of the first mobile phase solvent is increased over time in relation to the concentration of the second mobile phase solvent. In some embodiments, the concentration of the second mobile phase solvent is increased
over time in relation to the concentration of the first mobile phase solvent.

Those of ordinary skill in the art will recognize that the gradient used to elute the one or more metabolites may include any compatible mobile phase solvents useful for the separation of the sample having one or more metabolites into its component metabolites. For example, compatible solvents may include, but are not limited to, water, acetonitrile, methanol, ethanol, ethyl acetate, hexanes, and the like, which may optionally further include one or more additives. Compatible additives may include acids or bases, wherein the acids or bases may be selected from, but are not limited to, formic acid, acetic acid, trifluoroacetic acid, ammonium acetate, and the like. Likewise, one of ordinary skill in the art will recognize that the gradient may be run over any appropriate period of time with any appropriate flow rate sufficient to separate the sample having one or more metabolites into its component metabolites.

In some embodiments, the gradient includes water as a mobile phase solvent. In some embodiments, the gradient includes water as a mobile phase solvent, wherein the mobile phase solvent further includes trifluoroacetic acid as an additive. In some embodiments, the gradient includes water supplemented with 0.01% trifluoroacetic acid as a mobile phase solvent. In some embodiments, the gradient includes acetonitrile as a mobile phase solvent. In some embodiments, the gradient includes acetonitrile as a mobile phase solvent, wherein the mobile phase solvent further includes trifluoroacetic acid as an additive.

In some embodiments, the gradient includes acetonitrile supplemented with 0.01% trifluoroacetic acid as a mobile phase solvent.

In some embodiments, the gradient has an initial concentration of the second mobile phase solvent of about 40% and a final concentration of the second mobile phase solvent of about 100%. In some embodiments, the gradient has a runtime of about 15 minutes to about 45 minutes. In some embodiments, the gradient has a runtime of about 30 minutes. In some embodiments, the gradient has a flow rate of about 0.5 to about 1.5 mL/min. In some embodiments, the gradient has a flow rate of about 0.8 mL/min.

In some embodiments, the first metabolite profile is a high-performance liquid chromatography chromatogram.

In some embodiments, the high-performance liquid chromatography method, includes: subjecting the sample to a C18 column, wherein the C18 column has a diameter of 4.6 mm, a length of 100 mm, and a temperature of about 20° C. to about 40° C.; and eluting the one or more metabolites with a gradient having a first and second mobile phase solvent, wherein: the first mobile phase solvent includes water; the second mobile phase solvent includes acetonitrile; the gradient has an initial concentration of the second mobile phase solvent of about 40% and a final concentration of the second mobile phase solvent of about 100%; and the gradient has a runtime of about 30 minutes and a flow rate of about 0.8 mL/min.

In some embodiments, the one or more unique elements have one or more retention times selected from the group consisting of: 6.8 minutes, about 8.3 minutes, about 8.6 minutes, about 8.7 minutes, about 9.0 minutes, about 10.5 minutes, and about 12.1 minutes, wherein the retention times are determined via the aforementioned HPLC Method. In some embodiments, the one or more unique elements have one or more retention times selected from the group consisting of about 8.7 minutes, about 9.0 minutes, and about 12.1 minutes, wherein the retention times are determined via the aforementioned HPLC Method. In some embodiments, the one or more unique elements have one or more retention times selected from the group consisting of about 6.8 minutes, about 8.3 minutes, about 8.6 minutes, and about 10.5 minutes, wherein the retention times are determined via the aforementioned HPLC Method. Those of ordinary skill in the art will appreciate that retention times may vary slightly, for example from replicate to replicate as a result of variation in column or instrumentation performance. Accordingly, the aforementioned retention times should be understood to encompass retention times within ±0.2 minutes of the recited values. By way of example, the recited retention time of 8.7 minutes is equivalent to a retention time within the range of 8.5 minutes to 8.9 minutes.

In some embodiments, the method for identifying a microorganism that produces one or more metabolites that impart one or more beneficial properties to a plant further includes comparing the first metabolite profile to a second metabolite profile. In some embodiments, the second metabolite profile is obtained from a second sample having one or more metabolites from a second microorganism. In some embodiments, the second metabolite profile is obtained from a second sample having one or more metabolites from a second microorganism, wherein the second microorganism does not produce metabolites that impart the one or more beneficial properties to a plant. The second sample having one or more metabolites can be prepared from a supernatant sample of a culture that includes the second microorganism, a whole broth sample of a culture that includes the second microorganism, or an extract of a culture that includes the second microorganism. In some embodiments, the second sample having one or more metabolites is prepared from a supernatant sample of a culture that includes the second microorganism. The supernatant sample can be prepared by centrifuging a culture that includes the microorganism and separating the supernatant from pelleted cells and other solid components of the culture. In some embodiments, the second sample having one or more metabolites from the microorganism that produces metabolites that impart one or more beneficial traits to a plant is a whole broth sample of a culture that includes the microorganism. In some embodiments, the second sample having one or more metabolites from the microorganism that produces metabolites that impart one or more beneficial traits to a plant is an extract of a culture that includes the microorganism. The extract can be prepared by lysing the cultured cells using techniques known to those of ordinary skill in the art and subsequently separating the soluble extracts from insoluble cell debris and other components of the culture, such as by centrifugation, for example.

In some embodiments, the second metabolite profile is obtained by subjecting the first sample having one or more metabolites to an analytical technique to identify the elements that comprise the second sample. The analytical technique may be any technique known to one of ordinary skill in the art that is capable of identifying the component metabolites within the first sample having one or more metabolites. For example, the analytical technique may be, but is not limited to, a chemical separation, a chromatographic separation, nuclear magnetic resonance spectroscopy, mass spectrometry, or the like. In some embodiments, the second metabolite profile is obtained by subjecting the first sample having one or more metabolites to chromatographic separation. In some embodiments, obtaining the second metabolite profile includes subjecting the second sample having one or more metabolites to chromatographic separation, wherein subjecting the second sample having one or more metabolites to chromatographic separation includes subjecting the second sample to a high-performance liquid chromatography method. In some embodiments, the high-performance liquid chromatography method includes: subjecting the sample to a column; and eluting the one or more metabolites with a gradient of a first and second mobile phase solvent. The “gradient of the first and second mobile phase solvent” refers to changing the composition of a mixture of the first and second mobile phase solvent over time. In some embodiments, the concentration of the first mobile phase solvent is increase over time in relation to the concentration of the second mobile phase solvent. In some embodiments, the concentration of the second mobile phase solvent is increased over time in relation to the concentration of the first mobile phase solvent.

Those of ordinary skill in the art will recognize that the gradient used to elute the one or more metabolites may include any compatible mobile phase solvents useful for the separation of the sample having one or more metabolites into its component metabolites. For example, compatible solvents may include, but are not limited to, water, acetonitrile, methanol, ethanol, ethyl acetate, hexanes, and the like, which may optionally further include one or more additives. Compatible additives may include acids or bases, wherein the acids or bases may be selected from, but are not limited to, formic acid, acetic acid, trifluoroacetic acid, ammonium acetate, and the like. Likewise, one of ordinary skill in the art will recognize that the gradient may be run over any appropriate period of time with any appropriate flow rate sufficient to separate the sample having one or more metabolites into its component metabolites.

In some embodiments, the gradient includes water as a mobile phase solvent. In some embodiments, the gradient includes water as a mobile phase solvent, wherein the mobile phase solvent further includes trifluoroacetic acid as an additive. In some embodiments, the gradient includes water supplemented with 0.01% trifluoroacetic acid as a mobile phase solvent. In some embodiments, the gradient includes acetonitrile as a mobile phase solvent. In some embodiments, the gradient includes acetonitrile as a mobile phase solvent, wherein the mobile phase solvent further includes trifluoroacetic acid as an additive.

In some embodiments, the gradient includes acetonitrile supplemented with 0.01% trifluoroacetic acid as a mobile phase solvent.

In some embodiments, the gradient includes an initial concentration of the second mobile phase solvent of about 40% and a final concentration of the second mobile phase solvent of about 100%. In some embodiments, the gradient has a runtime of about 15 minutes to about 45 minutes. In some embodiments, the gradient has a runtime of about 30 minutes. In some embodiments, the gradient has a flow rate of about 0.5 to about 1.5 mL/min. In some embodiments, the gradient has a flow rate of about 0.8 mL/min.

In some embodiments, the second metabolite profile is a high-performance liquid chromatography chromatogram. In some embodiments, the second metabolite profile is a high-performance liquid chromatography chromatogram, wherein the chromatogram is obtained by subjecting the second sample having one or more metabolites to the same high-performance liquid chromatography method used to obtain the first metabolite profile.

In some embodiments, the second microorganism from which the second sample having one or more metabolites is prepared from can be cultured for 1 to 14 days in liquid media prior to obtaining the sample having one or more metabolites from the microorganism. In some embodiments, the microorganism may be cultured for 1 day, 2 days, 3 days, 4 days, 5 days, 6 days, 7 days, 8 days, 9 days, 10 days, 11 days, 12 days, 13 days, or 14 days prior to obtaining the sample having one or more metabolites. In some embodiments, the microorganism may be cultured for about 3 days to about 5 days prior to obtaining the sample having one or more metabolites. In some embodiments, the microorganism may be cultured for 4 days prior to obtaining the sample having one or more metabolites.

In some embodiments, the second microorganism from which the second sample having one or more metabolites is prepared from belongs to a genus selected from the group consisting of Bacillus, Pseudomonas, and Paenibacillus. In some embodiments the first and second microorganism from which the first and second samples having one or more metabolites are respectively prepared belong to a genus selected from Bacillus, Pseudomonas, and Paenibacillus. In some embodiments, the first and second microorganism from which the first and second samples having one or more metabolites are respectively prepared belong to the genus Bacillus.

In some embodiments, the method of identifying a microorganism that produces one or more metabolites that impart one or more beneficial traits to a plant further includes comparing the first metabolite profile to a second metabolite profile and selecting the microorganism that produces the one or more metabolites that impart one or more beneficial traits to a plant when the first metabolite profile has one or more unique elements, wherein the one or more unique elements identified in the first metabolite profile are absent from the second metabolite profile and at least one of the one or more unique elements corresponds to the one or more unique elements. By way of example, the first metabolite profile can be a high-performance liquid chromatography chromatogram that includes a number of peaks corresponding to one or more elements of the first sample having one or more metabolites. The first metabolite profile chromatogram can be compared to a second metabolite profile, which is a high-performance liquid chromatography chromatogram that includes a number of peaks corresponding to one or more elements of the second sample having one or more metabolites. The first metabolite profile chromatogram can include unique peaks that are absent from the second metabolite profile chromatogram, wherein the unique peaks correspond to one or more metabolites that impart a beneficial trait to the plant.

In another aspect, the present disclosure relates to a method of selecting a microbial strain or species that produces one or more metabolites that control one or more biotic stressors on or in a plant comprising: obtaining a sample comprising one or more metabolites; obtaining a metabolite profile from the first sample; and selecting the microbe as one that produces metabolites that control one or more biotic stressors on or in the plant when the metabolite profile comprises one or more lipopeptides having one or more retention times selected from the group consisting of 6.8 minutes, 8.3 minutes, 8.6 minutes, 8.7 minutes, 9.0 minutes, 10.5 minutes, and 12.1 minutes, wherein the retention times are determined via a high-performance liquid chromatography method comprising: subjecting the sample to a C18 column, wherein the C18 column has a diameter of 4.6 mm, a length of 100 mm, and a temperature of 25° C.; and eluting the one or more metabolites with a gradient comprising a first and second mobile phase solvent, wherein: the first mobile phase solvent comprises water; the second mobile phase solvent comprises acetonitrile; the gradient comprises an initial concentration of the second mobile phase solvent of 40% and a final concentration of the second mobile phase solvent of 100%; and the gradient comprises a runtime of 30 minutes and a flow rate of 0.8 mL/min.

In some embodiments, the one or more lipopeptides have one or more retention times selected from the group consisting of 8.7 minutes, 9.0 minutes, and 12.1 minutes. In some embodiments, the one or more lipopeptides have a retention time of 8.7 minutes. In some embodiments, the one or more lipopeptides have a retention time of 9.0 minutes. In some embodiments, the one or more lipopeptides have a retention time of 12.1 minutes. In some embodiments, the one or more lipopeptides have one or more retention times selected from the group consisting of 6.8 minutes, 8.3 minutes, 8.6 minutes, and 10.5 minutes. In some embodiments, the one or more lipopeptides have a retention time of 6.8 minutes. In some embodiments, the one or more lipopeptides have a retention time of 8.3 minutes. In some embodiments, the one or more lipopeptides have a retention time of 8.6 minutes. In some embodiments, the one or more lipopeptides have a retention time of 10.5 minutes. Those of ordinary skill in the art will appreciate that retention times may vary slightly, for example from replicate to replicate as a result of variation in column or instrumentation performance. Accordingly, the aforementioned retention times should be understood to encompass retention times within ±0.2 minutes of the recited values. By way of example, the recited retention time of 8.7 minutes is equivalent to a retention time within the range of 8.5 minutes to 8.9 minutes.

In some embodiments, the methods disclosed herein are useful for the identification of Bacillus species that produce a Category 2 metabolite profile, as described in Example 1 herein. In some embodiments, Bacillus species that produce a Category 2 metabolite profile include Bacillus amyloliquefaciens, Bacillus methylotrophicus, Bacillus tequilensis, and Bacillus velezensis.

In some embodiments, the methods disclosed herein are useful for the identification of Bacillus species that produce a Category 3 metabolite profile, as described in Example 2 herein. In some embodiments, Bacillus species that produce a Category 3 metabolite profile include Bacillus amyloliquefaciens, Bacillus methylotrophicus, and Bacillus velezensis.

### Microbial-Produced Compositions

In some cases, the microbes of the present disclosure may produce one or more compounds and/or have one or more activities, e.g., one or more of the following: production of a metabolite, production of a cyclic lipopeptide, production of a phytohormone such as auxin, production of acetoin, production of an antimicrobial compound, production of a siderophore, production of a polyketide, production of a phenazine, production of a cellulase, production of a pectinase, production of a chitinase, production of a glucanase, production of a xylanase, nitrogen fixation, or mineral phosphate solubilization.

For example, a microbe of the disclosure may produce a phytohormone selected from the group consisting of an auxin, a cytokinin, a gibberellin, ethylene, a brassinosteroid, and abscisic acid.

Thus, a “metabolite produced by” a microbe of the disclosure, is intended to capture any molecule (small molecule, vitamin, mineral, protein, nucleic acid, lipid, fat, carbohydrate, etc.) produced by the microbe. Often, the exact mechanism of action, whereby a microbe of the disclosure imparts a beneficial trait upon a given plant species is not known. It is hypothesized, that in some instances, the microbe is producing a metabolite that is beneficial to the plant. Thus, in some aspects, a cell-free or inactivated preparation of microbes is beneficial to a plant, as the microbe does not have to be alive to impart a beneficial trait upon the given plant species, so long as the preparation includes a metabolite that was produced by said microbe and which is beneficial to a plant.

In one embodiment, the microbes of the disclosure may produce auxin (e.g., indole-3-acetic acid (IAA)). Production of auxin can be assayed. Many of the microbes described herein may be capable of producing the plant hormone auxin indole-3-acetic acid (IAA) when grown in culture. Auxin plays a key role in altering the physiology of the plant, including the extent of root growth.

Therefore, in an embodiment, the microbes of the disclosure are present as a population disposed on the surface or within a tissue of a given plant species. The microbes may produce a composition, such as a metabolite, in an amount effective to cause a detectable increase in the amount of composition that is found on or within the plant, when compared to a reference plant not treated with the microbes or cell-free or inactive preparations of the disclosure. The composition produced by said microbial population may be beneficial to the plant species.

Such microbial-produced compositions may be present in the cell culture broth or medium/a in which the microbes are grown, or may encompass an exudate produced by the microbes. As used herein, “exudate” refers to one or more compositions excreted by or extracted from one or more microbial cell(s). As used herein, “broth” refers to the collective composition of a cell culture medium after microbial cells are placed in the medium. The composition of the broth may change over time, during different phases of microbial growth and/or development. Broth and/or exudate may improve the traits of plants with which they become associated.

### Cyclic Lipopeptides (CLPs)

Bacterial-produced CLPs are known to have fungicidal and/or bactericidal activity (see, for example, Malfanova et al., “Cyclic lipopeptide profile of the plant-beneficial endophytic bacterium Bacillus subtilis HC8”, Archives of Microbiology Volume 194, pages 893-899 (2012)). CLPs are amphiphilic molecules, comprising a fatty acid tail linked to a short oligopeptide which form a macrocylic ring structure. These CLPs include several major classes of compounds, such as iturins, fengycins, and surfactins.

Iturins are a family of lipopeptides extracted from the culture media of various strains of Bacillus subtilis. These amphiphilic compounds are characterized by a peptide ring of seven amino acid residues including an invariable D-Tyr2, with the constant chiral sequence LDDLLDL closed by a C14-C17 aliphatic beta-amino acid. (Maget-Dana and Peypoux, “Iturins, a special class of pore-forming lipopeptides: biological and physicochemical properties”, Toxicology 87(1-3):151-74, 1994.)

Fengycin lipopeptides are partially cyclic, and the lactone bond formation occurs between the third amino acid 1-Tyr and the last amino acid 1-Ile (Samel et al., 2006). It consists of an anionic cyclic decapeptide with a β-hydroxy fatty acid attached at the N-terminus.

Surfactin is an anionic cyclic lipopeptide is constituted by a heptapeptide interlinked with a β-hydroxy fatty acid. Due to its amhipathic nature surfactin incorporates into the phospholipid bilayer and induces permeabilization and perturbation of target cells. (Seydlova and Svobodova, “Review of Surfactin chemical properties and the potential biomedical applications”, Central European Journal of Medicine Vol 3 pp 123-133, 2008.)

### Polyketides

Polyketides are structurally diverse and biologically active secondary metabolites produced by bacteria and other organisms. Polyketides are a large family of compounds, which are formed through the condensation of acyl-thioester units such as malonyl-CoA and methylmalonyl-CoA to yield metabolites with diverse structures and biological activities. Examples of polyketides include, but are not limited to: macrolides (e.g., difficidin, macrolactin), ansamycins, polyenes (e.g., bacillaene), polyethers, tetracyclines, acetogenins.

There are three types of polyketide synthases (PKSs) recognized in bacteria. Multimodular PKSs consist of one or more large multidomain polypeptides where the growing polyketide chain is sequentially passed from one active site to the next. Depending on the nature of their constituent catalytic domains, these megasynthases generate chemical variety and complexity in a stepwise fashion. Iterative PKSs comprise a single set of catalysts that assemble a polyketide of controlled chain length through repetitive use of active sites. In both cases, the nascent polyketide product is frequently acted on by further tailoring enzymes to generate the antibiotic. A third type of PKS (called type III PKSs) is different, in that the growing polyketide chain is never directly attached to a protein. (Ridley et al., “Evolution of polyketide synthases in bacteria”, PNAS Vol 105 Issue 12, pp 4595-4600, 2008.)

### Agricultural Compositions

In some embodiments, the microbes of the disclosure are combined with agricultural compositions. Agricultural compositions generally refer to organic and inorganic compounds that can include compositions that promote the cultivation of the microbe and/or the plant element; compositions involved in formulation of microbes for application to plant elements (for example, but not limited to: wetters, compatibilizing agents (also referred to as “compatibility agents”), antifoam agents, cleaning agents, sequestering agents, drift reduction agents, neutralizing agents and buffers, corrosion inhibitors, dyes, odorants, spreading agents (also referred to as “spreaders”), penetration aids (also referred to as “penetrants”), sticking agents (also referred to as “stickers” or “binders”), dispersing agents, thickening agents (also referred to as “thickeners”), stabilizers, emulsifiers, freezing point depressants, antimicrobial agents, and the like); compositions involved in conferring protection to the plant element or plant (for example, but not limited to: pesticides, nematicides, fungicides, bactericides, herbicides, and the like); as well as other compositions that may be of interest for the particular application.

In some embodiments, the compositions of the present disclosure are solid. Where solid compositions are used, it may be desired to include one or more carrier materials with the active isolated microbe or consortia. In some embodiments, the present disclosure teaches the use of carriers including, but not limited to: mineral earths such as silicas, silica gels, silicates, talc, kaolin, attaclay, limestone, chalk, loess, clay, dolomite, diatomaceous earth, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or compositions of these.

### Growth Compositions

In some embodiments, a composition is provided to the microbe and/or the plant element that promotes the growth and development. Exemplary compositions include liquid (such as broth, media) and/or solid (such as soil, nutrients). Various organic or inorganic compounds may be added to the growth composition to facilitate the health of the microbe, alone or in combination with the plant element, for example but not limited to: amino acids, vitamins, minerals, carbohydrates, simple sugars, lipids.

### Formulation Compositions

One or more compositions, in addition to the microbe(s) or microbial-produced composition, may be combined for various application, stability, activity, and/or storage reasons. The additional compositions may be referred to as “formulation components”.

In some embodiments, the compositions of the present disclosure are liquid. Thus in some embodiments, the present disclosure teaches that the compositions disclosed herein can include compounds or salts such as monoethanolamine salt, sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate.

In some embodiments, the present disclosure teaches that compositions can include binders such as: polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or compositions of these; lubricants such as magnesium stearate, sodium stearate, talc or polyethylene glycol, or compositions of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or compositions of these.

In some embodiments, the compositions comprise surface-active agents. In some embodiments, the surface-active agents are added to liquid agricultural compositions. In other embodiments, the surface-active agents are added to solid formulations, especially those designed to be diluted with a carrier before application. Thus, in some embodiments, the compositions comprise surfactants. Surfactants are sometimes used, either alone or with other additives, such as mineral or vegetable oils as adjuvants to spray-tank mixes to improve the biological performance of the microbes on the target. The types of surfactants used for bioenhancement depend generally on the nature and mode of action of the microbes. The surface-active agents can be anionic, cationic, or nonionic in character, and can be employed as emulsifying agents, wetting agents, suspending agents, or for other purposes. In some embodiments, the surfactants are non-ionics such as: alky ethoxylates, linear aliphatic alcohol ethoxylates, and aliphatic amine ethoxylates. Surfactants conventionally used in the art of formulation and which may also be used in the present formulations are described, in McCutcheon's Detergents and Emulsifiers Annual, MC Publishing Corp., Ridgewood, N.J., 1998, and in Encyclopedia of Surfactants, Vol. I-III, Chemical Publishing Co., New York, 1980-81. In some embodiments, the present disclosure teaches the use of surfactants including alkali metal, alkaline earth metal or ammonium salts of aromatic sulfonic acids, for example, ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or compositions of these.

In some embodiments, the present disclosure teaches other suitable surface-active agents, including salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutyl-naphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, particularly methyl esters.

In some embodiments, the compositions comprise wetting agents. A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank or other vessel to reduce the wetting time of wettable powders and to improve the penetration of water into water-dispersible granules. In some embodiments, examples of wetting agents used in the compositions of the present disclosure, including wettable powders, suspension concentrates, and water-dispersible granule formulations are: sodium lauryl sulphate; sodium dioctyl sulphosuccinate; alkyl phenol ethoxylates; and aliphatic alcohol ethoxylates.

In some embodiments, the compositions of the present disclosure comprise dispersing agents. A dispersing agent is a substance which adsorbs onto the surface of particles and helps to preserve the state of dispersion of the particles and prevents them from re-aggregating. In some embodiments, dispersing agents are added to compositions of the present disclosure to facilitate dispersion and suspension during manufacture, and to ensure the particles redisperse into water in a spray tank. In some embodiments, dispersing agents are used in wettable powders, suspension concentrates, and water-dispersible granules. Surfactants that are used as dispersing agents have the ability to adsorb strongly onto a particle surface and provide a charged or steric barrier to re-aggregation of particles. In some embodiments, the most commonly used surfactants are anionic, non-ionic, or mixtures of the two types.

In some embodiments, for wettable powder formulations, the most common dispersing agents are sodium lignosulphonates. In some embodiments, suspension concentrates provide very good adsorption and stabilization using polyelectrolytes, such as sodium naphthalene sulphonate formaldehyde condensates. In some embodiments, tristyrylphenol ethoxylate phosphate esters are also used. In some embodiments, such as alkylarylethylene oxide condensates and EO-PO block copolymers are sometimes combined with anionics as dispersing agents for suspension concentrates.

In some embodiments, the compositions of the present disclosure comprise polymeric surfactants. In some embodiments, the polymeric surfactants have very long hydrophobic ‘backbones’ and a large number of ethylene oxide chains forming the ‘teeth’ of a ‘comb’ surfactant. In some embodiments, these high molecular weight polymers can give very good long-term stability to suspension concentrates, because the hydrophobic backbones have many anchoring points onto the particle surfaces. In some embodiments, examples of dispersing agents used in compositions of the present disclosure are: sodium lignosulphonates; sodium naphthalene sulphonate formaldehyde condensates; tristyrylphenol ethoxylate phosphate esters; aliphatic alcohol ethoxylates; alky ethoxylates; EO-PO block copolymers; and graft copolymers.

In some embodiments, the compositions of the present disclosure comprise emulsifying agents. An emulsifying agent is a substance, which stabilizes a suspension of droplets of one liquid phase in another liquid phase. Without the emulsifying agent the two liquids would separate into two immiscible liquid phases. In some embodiments, the most commonly used emulsifier blends include alkylphenol or aliphatic alcohol with 12 or more ethylene oxide units and the oil-soluble calcium salt of dodecylbenzene sulphonic acid. A range of hydrophile-lipophile balance (“HLB”) values from 8 to 18 will normally provide good stable emulsions. In some embodiments, emulsion stability can sometimes be improved by the addition of a small amount of an EO-PO block copolymer surfactant.

In some embodiments, the compositions of the present disclosure comprise solubilizing agents. A solubilizing agent is a surfactant, which will form micelles in water at concentrations above the critical micelle concentration. The micelles are then able to dissolve or solubilize water-insoluble materials inside the hydrophobic part of the micelle. The types of surfactants usually used for solubilization are non-ionics: sorbitan monooleates; sorbitan monooleate ethoxylates; and methyl oleate esters.

In some embodiments, the compositions of the present disclosure comprise organic solvents. Organic solvents are used mainly in the formulation of emulsifiable concentrates, ULV formulations, and to a lesser extent granular formulations. Sometimes mixtures of solvents are used. In some embodiments, the present disclosure teaches the use of solvents including aliphatic paraffinic oils such as kerosene or refined paraffins. In other embodiments, the present disclosure teaches the use of aromatic solvents such as xylene and higher molecular weight fractions of C9 and C10 aromatic solvents. In some embodiments, chlorinated hydrocarbons are useful as co-solvents to prevent crystallization of pesticides when the formulation is emulsified into water. Alcohols are sometimes used as co-solvents to increase solvent power.

In some embodiments, the compositions comprise gelling agents. Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. In some embodiments, the compositions comprise one or more thickeners including, but not limited to: montmorillonite, e.g., bentonite; magnesium aluminum silicate; and attapulgite. In some embodiments, the present disclosure teaches the use of polysaccharides as thickening agents. The types of polysaccharides most commonly used are natural extracts of seeds and seaweeds or synthetic derivatives of cellulose. Some embodiments utilize xanthan and some embodiments utilize cellulose. In some embodiments, the present disclosure teaches the use of thickening agents including, but are not limited to: guar gum; locust bean gum; carrageenin; alginates; methyl cellulose; sodium carboxymethyl cellulose (SCMC); hydroxyethyl cellulose (HEC). In some embodiments, the present disclosure teaches the use of other types of anti-settling agents such as modified starches, polyacrylates, polyvinyl alcohol, and polyethylene oxide. Another good anti-settling agent is xanthan gum.

In some embodiments, the presence of surfactants, which lower interfacial tension, can cause water-based formulations to foam during mixing operations in production and in application through a spray tank. Thus, in some embodiments, in order to reduce the tendency to foam, anti-foam agents are often added either during the production stage or before filling into bottles/spray tanks. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

In some embodiments, the compositions comprise a preservative.

In some embodiments, the compositions may be formulated as: a soil drench, a foliar spray, a dip treatment, an in-furrow treatment, a soil amendment, granules, a broadcast treatment, a post-harvest disease control treatment, or a seed treatment. In some embodiments, the compositions may be applied alone in or in rotation spray programs with other agricultural products.

In some embodiments, the compositions may be compatible with tank mixing. In some embodiments, the compositions may be compatible with tank mixing with other agricultural products. In some embodiments, the compositions may be compatible with equipment used for ground, aerial, and irrigation applications.

In some embodiments, the compositions may be applied to genetically modified seeds or plants.

### Protective Compositions

Further, the individual microbes, or microbial consortia, or microbial communities, developed according to the disclosed methods can be combined with known actives available in the agricultural space, such as: pesticide, herbicide, bactericide, fungicide, insecticide, virucide, miticide, nematicide, acaricide, plant growth regulator, rodenticide, anti-algae agent, biocontrol or beneficial agent. Further, the microbes, microbial consortia, or microbial communities developed according to the disclosed methods can be combined with known fertilizers. Such combinations may exhibit synergistic properties. Further still, the individual microbes, or microbial consortia, or microbial communities, developed according to the disclosed methods can be combined with inert ingredients. Also, in some aspects, the disclosed microbes are combined with biological active agents.

In some embodiments, the individual microbes, or microbial consortia, or microbial communities, developed according to the disclosed methods can be combined with biopesticides that function as an herbicide, bactericide, fungicide, insecticide, virucide, miticide, nematicide, acaricide, rodenticide, and/or anti-algae agent. Such biopesticides may be, but are not limited to, macrobial organisms (e.g., beneficial nematodes and the like), microbial organisms (e.g., Serenade®, Bacillus thuringiensis, and the like), plant extracts (e.g., Timorex Gold and the like), biochemical (e.g., insect pheromones and the like), and/or minerals and oils (e.g., canola oil and the like).

### Pesticides and Biopesticides

In some embodiments, the compositions of the present disclosure comprise pesticides, used in combination with the taught microbes. In some embodiments, the compositions of the present disclosure comprise biopesticides, used in combination with the taught microbes.

In some embodiments, the individual microbes, or microbial consortia, or microbial communities, developed according to the disclosed methods can be combined with known pesticides in the agricultural space, such as: pesticides that function as an herbicide, bactericide, fungicide, insecticide, virucide, miticide, nematicide, acaricide, rodenticide, and/or anti-algae agent.

In some embodiments, the individual microbes, or microbial consortia, or microbial communities, developed according to the disclosed methods can be combined with known biopesticides in the agricultural space, such as: biopesticides that function as an herbicide, bactericide, fungicide, insecticide, virucide, miticide, nematicide, acaricide, rodenticide, and/or anti-algae agent.

As used herein, the term “control” refers to the regulation or management of a species that is recognized as having a negative effect on an agricultural process or product, such as a plant. The control of the species may be achieved through the use of chemical or biological agents. The control of the species may involve the eradication of the species from the agricultural process or product, the reduction of the population of the species to a level that the species no longer has a negative effect on the agricultural process or product, or the protection of the agricultural process or product from the species.

As used herein, the “control of one or more phytopathogens on or in a plant” refers to the control of a phytopathogen species that has infected or otherwise colonized the plant. For example, a chemical or biological agent may be applied to the plant, or media in which the plant is growing, to eradicate or reduce the population of the phytopathogen on, in, or around the plant. The population reduction may be to a level sufficient to prevent negative effects from the infection or colonization of the plant by the phytopathogen.

Alternatively, the plant may be protected from infection or colonization by the phytopathogen. By way of example, an applied biological or chemical agent may prevent the infection or colonization of the plant by the phytopathogen, for example by killing or otherwise inactivating the phytopathogen before the infection or colonization is established on, in, or around the plant.

As used herein, the term “biocontrol” is equivalent to the term “biological control” and refers to the use of a biological organism, or a product thereof, in the control of a species recognized as having a negative effect on an agricultural process or product, such as a plant. For example, the biocontrol organism may involve the eradication of the species from the plant, the reduction of the population of the species to a level that the species no longer has a negative effect on the agricultural process or product, or the protection of the agricultural process or product from the species.

As used herein, the “biocontrol of one or more phytopathogens on or in a plant” refers to the use of a biological organism, or a product thereof, in the biocontrol of a phytopathogen species that has infected or otherwise colonized the plant. For example, the biological organism may be applied to the plant, or media in which the plant is growing, to eradicate or reduce the population of the phytopathogen on, in, or around the plant. The population reduction may be to a level sufficient to prevent negative effects from the infection or colonization of the plant by the phytopathogen. Alternatively, the plant may be protected from infection or colonization by the phytopathogen. By way of example, the applied biological organism, or a product thereof, may prevent the infection or colonization of the plant by the phytopathogen, for example by killing or otherwise inactivating the phytopathogen before the infection or colonization is established on, in, or around the plant.

For example, in some embodiments, the present disclosure teaches compositions comprising one or more of the following active ingredients including: macrobial organisms (e.g., beneficial nematodes and the like), microbial organisms (e.g., Serenade, Bt, and the like), plant extracts (e.g., Timorex Gold and the like), biochemical (e.g., insect pheromones and the like), and/or minerals and oils (e.g., canola oil).

In some embodiments, the individual microbes, or microbial consortia, or microbial communities, developed according to the disclosed methods can be combined with an herbicide selected from the group consisting of: an acetamide selected from the group consisting of acetochlor, alachlor, butachlor, dimethachlor, dimethenamid, flufenacet, mefenacet, metolachlor, metazachlor, napropamide, naproanilide, pethoxamid, pretilachlor, propachlor, and thenylchlor; an amino acid derivative selected from the group consisting of bilanafos, glufosinate, and sulfosate; an aryloxyphenoxypropionate selected from the group consisting of clodinafop, cyhalofop-butyl, fenoxaprop, fluazifop, haloxyfop, metamifop, propaquizafop, quizalofop, and quizalo-fop-P-tefuryl; diquat and paraquat; a (thio)carbamate selected from the group consisting of asulam, butylate, carbetamide, desmedipham, dimepiperate, eptam (EPTC), esprocarb, molinate, orbencarb, phenmedipham, prosulfocarb, pyributicarb, thiobencarb, and triallate; a cyclohexanedione selected from the group consisting of butroxydim, clethodim, cycloxydim, profoxydim, sethoxydim, tepraloxydim, and tralkoxydim; a dinitroaniline selected from the group consisting of benfluralin, ethalfluralin, oryzalin, pendimethalin, prodiamine, and trifluralin; a diphenyl ether selected from the group consisting of acifluorfen, aclonifen, bifenox, diclofop, ethoxyfen, fomesafen, lactofen, and oxyfluorfen; a hydroxybenzonitrile selected from the group consisting of bomoxynil, dichlobenil, and ioxynil; an imidazolinone selected from the group consisting of imazamethabenz, imazamox, imazapic, imazapyr, imazaquin, and imazethapyr; a phenoxy acetic acid selected from the group consisting of clomeprop, 2,4-dichlorophenoxyacetic acid (2,4-D), 2,4-DB, dichlorprop, MCPA, MCPA-thioethyl, MCPB, and Mecoprop; a pyrazine selected from the group consisting of chloridazon, flufenpyr-ethyl, fluthiacet, norflurazon, and pyridate; a pyridine selected from the group consisting of aminopyralid, clopyralid, diflufenican, dithiopyr, fluridone, fluroxypyr, picloram, picolinafen, and thiazopyr; a sulfonyl urea selected from the group consisting of amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, and 14(2-chloro-6-propyl-imidazol[1,2]-blpyridazin-3-yl)sulfonyl)-3-(4,6-dimethoxy-pyrimidin-2-yl)urea; a triazine selected from the group consisting of ametryn, atrazine, cyanazine, a dimethametryn, ethiozin, hexazinone, metamitron, metribuzin, prometryn, simazine, terbuthylazine, terbutryn, and triaziflam; a urea compound selected from the group consisting of chlorotoluron, daimuron, diuron, fluometuron, isoproturon, linuron, methabenzthiazuron, and tebuthiuron; an acetolactate synthase inhibitor selected from the group consisting of bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, and pyroxsulam; and a compound selected from the group consisting of amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclol[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxyl]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

In some embodiments, the individual microbes, or microbial consortia, or microbial communities, developed according to the disclosed methods can be combined with an insecticide selected from the group consisting of: an organo(thio)phosphate selected from the group consisting of acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon; a carbamate selected from the group consisting of alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate; a pyrethroid selected from the group consisting of allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, taufluvalinate, tefluthrin, tetramethrin, tralomethrin, transfluthrin, profluthrin, and dimefluthrin; an insect growth regulator selected from the group consisting of a) a chitin synthesis inhibitor wherein said chitin synthesis inhibitor is a benzoylurea selected from the group consisting of chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, and clofentazine; b) an ecdysone antagonist selected from the group consisting of halofenozide, methoxyfenozide, tebufenozide, and azadirachtin; c) a juvenoid selected from the group consisting of pyriproxyfen, methoprene, and fenoxycarb; or d) a lipid biosynthesis inhibitor selected from the group consisting of spirodiclofen, spiromesifen, and spirotetramat; a nicotinic receptor agonist/antagonist compound selected from the group consisting of clothianidin, dinotefuran, imidacloprid, thiamethoxam, nitenpyram, acetamiprid, thiacloprid, and 1-(2-chloro-thiazol-5-ylmethyl)-2-nitrimino-3,5-dimethyl-[1,3,5]triazinane; a GABA antagonist compound selected from the group consisting of endosulfan, ethiprole, fipronil, vaniliprole, pyrafluprole, pyriprole, and 5-amino-1-(2,6-dichloro-4-methyl-phenyl)-4-sulfinamoyl-1H-pyrazole-3-carbothioic acid amide; a macrocyclic lactone insecticide selected from the group consisting of abamectin, emamectin, milbemectin, lepimectin, spinosad, and spinetoram; a mitochondrial electron transport inhibitor (METI) I acaricide selected from the group consisting of fenazaquin, pyridaben, tebufenpyrad, tolfenpyrad, and flufenerim; a METI II and III compound selected from the group consisting of acequinocyl, fluacyprim, and hydramethylnon; chlorfenapyr; an oxidative phosphorylation inhibitor selected from the group consisting of cyhexatin, diafenthiuron, fenbutatin oxide, and propargite; cryomazine; piperonyl butoxide; a sodium channel blocker selected from the group consisting of indoxacarb and metaflumizone; and a compound selected from the group consisting of benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon.

In some embodiments, the present invention teaches a synergistic use of the presently disclosed microbes or microbial consortia with known pesticides in the agricultural space, such as: pesticides that function as an herbicide, bactericide, fungicide, insecticide, virucide, miticide, nematicide, acaricide, rodenticide, and/or anti-algae agent.

In some embodiments, the present invention teaches a synergistic use of the presently disclosed microbes or microbial consortia with known biopesticides in the agricultural space, such as: biopesticides that function as an herbicide, bactericide, fungicide, insecticide, virucide, miticide, nematicide, acaricide, rodenticide, and/or anti-algae agent.

In some embodiments, when the microbe or microbial consortia identified according to the taught methods is combined with a pesticide one witnesses an additive effect on a plant phenotypic trait of interest. In other embodiments, when the microbe or microbial consortia identified according to the taught methods is combined with a pesticide one witness a synergistic effect on a plant phenotypic trait of interest.

In some embodiments, when the microbe or microbial consortia identified according to the taught methods is combined with a biopesticide one witnesses an additive effect on a plant phenotypic trait of interest. In other embodiments, when the microbe or microbial consortia identified according to the taught methods is combined with a biopesticide one witness a synergistic effect on a plant phenotypic trait of interest.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agriculturally active pesticide compounds and also agricultural auxiliary pesticide compounds.

The isolated microbes and consortia of the present disclosure can synergistically increase the effectiveness of agriculturally active biopesticide compounds and also agricultural auxiliary biopesticide compounds.

### Plant Growth Regulators and Biostimulants

In some embodiments, the compositions of the present disclosure comprise plant growth regulators and/or biostimulants, used in combination with the taught microbes.

In some embodiments, the individual microbes, or microbial consortia, or microbial communities, developed according to the disclosed methods can be combined with known plant growth regulators in the agricultural space, such as: auxins, gibberellins, cytokinins, ethylene generators, growth inhibitors, and growth retardants.

For example, in some embodiments, the present disclosure teaches compositions comprising one or more of the following active ingredients including: ancymidol, butralin, alcohols, chloromequat chloride, cytokinin, daminozide, ethepohon, flurprimidol, giberrelic acid, gibberellin mixtures, indole-3-butryic acid (IBA), maleic hydrazide, mefludide, mepiquat chloride, mepiquat pentaborate, naphthalene-acetic acid (NAA), 1-napthaleneacetemide, (NAD), n-decanol, placlobutrazol, prohexadione calcium, trinexapac-ethyl, uniconazole, salicylic acid, abscisic acid, ethylene, brassinosteroids, jasmonates, polyamines, nitric oxide, strigolactones, or karrikins among others.

In some embodiments, the individual microbes, or microbial consortia, or microbial communities, developed according to the disclosed methods can be combined with seed inoculants known in the agricultural space, such as: QUICKROOTS®, VAULT®, RHIZO-STICK®, NODULATOR®, DORMAL®, SABREX®, among others. In some embodiments, a Bradyrhizobium inoculant is utilized in combination with any single microbe or microbial consortia disclosed here. In particular aspects, a synergistic effect is observed when one combines one of the aforementioned inoculants, e.g., QUICKROOTS® or Bradyrhizobium, with a microbe or microbial consortia as taught herein.

In some embodiments, the compositions of the present disclosure comprise a plant growth regulator, which contains: kinetin, gibberellic acid, and indole butyric acid, along with copper, manganese, and zinc.

In some embodiments, the present disclosure teaches compositions comprising one or more commercially available plant growth regulators, including but not limited to: Abide@, A-Rest®, Butralin®, Fair®, Royaltac M®, Sucker-Plucker®, Off-Shoot®, Contact-85®, Citadel®, Cycocel®, E-Pro®, Conklin®, Culbac®, Cytoplex®, Early Harvest®, Foli-Zyme®, Goldengro®, Happygro®, Incite®, Megagro®, Ascend®, Radiate®, Stimulate®, Suppress®, Validate®, X-Cyte®, B-Nine®, Compress®, Dazide®, Boll Buster®, BollD®, Cerone®, Cotton Quik®, Ethrel®, Finish®, Flash®, Florel®, Mature®, MFX®, Prep®, Proxy®, Quali-Pro®, SA-50®, Setup®, Super Boll®, Whiteout®, Cutless®, Legacy®, Mastiff®, Topflor®, Ascend®, Cytoplex®, Ascend®, Early Harvest®, Falgro®, Florgib®, Foli-Zyme®, GA3®, GibGro®, Green Sol®, Incite®, N-Large®, PGR IV®, Pro-Gibb®, Release®, Rouse®, Ryzup®, Stimulate®, BVB®, Chrysal®, Fascination®, Procone®, Fair®, Rite-Hite®, Royal®, Sucker Stuff®, Embark®, Sta-Lo®, Pix®, Pentia®, DipN Grow®, Goldengro®, Hi-Yield®, Rootone®, Antac®, FST-7®, Royaltac®, Bonzi®, Cambistat®, Cutdown®, Downsize®, Florazol®, Paclo®, Paczol®, Piccolo®, Profile®, Shortstop®, Trimmit®, Turf Enhancer®, Apogee®, Armor Tech®, Goldwing®, Governor®, Groom®, Legacy®, Primeraone®, Primo®, Provair®, Solace®, T-Nex®, T-Pac®, Concise®, and Sumagic®.

In some embodiments, the present invention teaches a synergistic use of the presently disclosed microbes or microbial consortia with plant growth regulators and/or stimulants such as phytohormones or chemicals that influence the production or disruption of plant growth regulators.

In some embodiments, the present invention teaches that phytohormones can include: Auxins (e.g., Indole acetic acid IAA), Gibberellins, Cytokinins (e.g., Kinetin), Abscisic acid, Ethylene (and its production as regulated by ACC synthase and disrupted by ACC deaminase).

In some embodiments, the individual microbes, or microbial consortia, or microbial communities, developed according to the disclosed methods can be combined with biostimulants. Such biostimulants may be, but are not limited to, microbial organisms, plant extracts, seaweeds, acids, biochar, and the like.

In some embodiments, the individual microbes, or microbial consortia, or microbial communities, developed according to the disclosed methods can be combined with fertilizers, which may be organic (e.g., manure, blood, fish, and the like), nitrogen-based (e.g., nitrate, ammonium, urea, and the like), phosphate, and potassium. Such fertilizers may also contain micronutrients including, but not limited to, sulfur, iron, zinc, and the like.

In some embodiments, the present invention teaches additional plant-growth promoting chemicals that may act in synergy with the microbes and microbial consortia disclosed herein, such as: humic acids, fulvic acids, amino acids, polyphenols and protein hydrolysates.

Thus, in some embodiments, the disclosure provides for the application of the taught microbes in combination with Ascend® upon any crop. Further, the disclosure provides for the application of the taught microbes in combination with Ascend® upon any crop and utilizing any method or application rate.

In some embodiments, the present disclosure teaches compositions with biostimulants.

As used herein, the term “biostimulant” refers to any substance that acts to stimulate the growth of microorganisms that may be present in soil or other plant growing medium.

The level of microorganisms in the soil or growing medium is directly correlated to plant health. Microorganisms feed on biodegradable carbon sources, and therefore plant health is also correlated with the quantity of organic matter in the soil. While fertilizers provide nutrients to feed and grow plants, in some embodiments, biostimulants provide biodegradable carbon, e.g., molasses, carbohydrates, e.g., sugars, to feed and grow microorganisms. Unless clearly stated otherwise, a biostimulant may comprise a single ingredient, or a combination of several different ingredients, capable of enhancing microbial activity or plant growth and development, due to the effect of one or more of the ingredients, either acting independently or in combination.

In some embodiments, biostimulants are compounds that produce non-nutritional plant growth responses. In some embodiments, many important benefits of biostimulants are based on their ability to influence hormonal activity. Hormones in plants (phytohormones) are chemical messengers regulating normal plant development as well as responses to the environment. Root and shoot growth, as well as other growth responses are regulated by phytohormones. In some embodiments, compounds in biostimulants can alter the hormonal status of a plant and exert large influences over its growth and health. Thus, in some embodiments, the present disclosure teaches sea kelp, humic acids, fulvic acids, and B Vitamins as common components of biostimulants. In some embodiments, the biostimulants of the present disclosure enhance antioxidant activity, which increases the plant's defensive system. In some embodiments, vitamin C, vitamin E, and amino acids such as glycine are antioxidants contained in biostimulants.

In other embodiments, biostimulants may act to stimulate the growth of microorganisms that are present in soil or other plant growing medium. Prior studies have shown that when certain biostimulants comprising specific organic seed extracts (e.g., soybean) were used in combination with a microbial inoculant, the biostimulants were capable of stimulating growth of microbes included in the microbial inoculant. Thus, in some embodiments, the present disclosure teaches one or more biostimulants that, when used with a microbial inoculant, is capable of enhancing the population of both native microbes and inoculant microbes. For a review of some popular uses of biostimulants, please see Calvo et al., 2014, Plant Soil 383:3-41.

Combinations of Plant Elements, Microbes, and/or Compositions

In some embodiments, the present disclosure teaches that the individual microbes, or microbial consortia, or microbial communities, or a composition produced from any of the preceding, or any combination of the preceding, may be applied to a plant element, optionally in combination with any agricultural composition, for the improvement of a plant phenotype.

Isolated microbes or communities or consortia (generally “microbes” or “microbe”, interchangeably) may be applied to a heterologous plant element, creating a synthetic combination. Microbes are considered heterologous to a plant element if they are not normally associated with the plant element in nature, or if found, are applied in amounts different than that found in nature. In some embodiments, the microbes may be found naturally in one part of a plant but not another, and introduction of the microbes to another part of the plant is considered a heterologous association.

It is further contemplated that the microbe, either isolated or in combination with a plant or plant element, may be further associated with one or more agricultural compositions, such as those described above.

Synthetic combinations of microbes and plant elements, microbes and agricultural compositions, and microbes and plant elements and compositions are contemplated (generally “synthetic compositions”, compositions that comprise components not typically found associated in nature).

### Plant Element Treatments

In some embodiments, the present disclosure also concerns the discovery that treating plant elements before they are sown or planted with a combination of one or more of the microbes or compositions of the present disclosure can enhance a desired plant trait, e.g., plant growth, plant health, and/or plant resistance to pests.

Thus, in some embodiments, the present disclosure teaches the use of one or more of the microbes or microbial consortia as plant element treatments. The plant element treatment can be a plant element coating applied directly to an untreated and “naked” plant element. However, the plant element treatment can be a plant element overcoat that is applied to a plant element that has already been coated with one or more previous plant element coatings or plant element treatments. The previous plant element treatments may include one or more active compounds, either chemical or biological, and one or more inert ingredients.

The term “plant element treatment” generally refers to application of a material to a plant element prior to or during the time it is planted in soil. Plant element treatment with microbes, and other compositions of the present disclosure, has the advantages of delivering the treatments to the locus at which the plant elements are planted shortly before germination of the plant element and emergence of a plant element.

In other embodiments, the present disclosure also teaches that the use of plant element treatments minimizes the amount of microbe or agricultural composition that is required to successfully treat the plants, and further limits the amount of contact of workers with the microbes and compositions compared to application techniques such as spraying over soil or over emerging plant element.

Moreover, in some embodiments, the present disclosure teaches that the microbes disclosed herein are important for enhancing the early stages of plant life (e.g., within the first thirty days following emergence of the plant element). Thus, in some embodiments, delivery of the microbes and/or compositions of the present disclosure as a plant element treatment places the microbe at the locus of action at a critical time for its activity.

In some embodiments, the microbial compositions of the present disclosure are formulated as a plant element treatment. In some embodiments, it is contemplated that the plant elements can be substantially uniformly coated with one or more layers of the microbes and/or compositions disclosed herein, using conventional methods of mixing, spraying, or a combination thereof through the use of treatment application equipment that is specifically designed and manufactured to accurately, safely, and efficiently apply plant element treatment products to plant elements. Such equipment uses various types of coating technology such as rotary coaters, drum coaters, fluidized bed techniques, spouted beds, rotary mists, or a combination thereof. Liquid plant element treatments such as those of the present disclosure can be applied via either a spinning “atomizer” disk or a spray nozzle, which evenly distributes the plant element treatment onto the plant element as it moves though the spray pattern. In aspects, the plant element is then mixed or tumbled for an additional period of time to achieve additional treatment distribution and drying.

The plant elements can be primed or unprimed before coating with the microbial compositions to increase the uniformity of germination and emergence. In an alternative embodiment, a dry powder formulation can be metered onto the moving plant element and allowed to mix until completely distributed.

In some embodiments, the plant elements have at least part of the surface area coated with a microbiological composition, according to the present disclosure. In some embodiments, a plant element coat comprising the microbial composition is applied directly to a naked plant element. In some embodiments, a plant element overcoat comprising the microbial composition is applied to a plant element that already has a plant element coat applied thereon. In some aspects, the plant element may have a plant element coat comprising, e.g., clothianidin and/or Bacillus firmus-I-1582, upon which the present composition will be applied on top of, as a plant element overcoat. In some aspects, the taught microbial compositions are applied as a plant element overcoat to plant elements that have already been treated with PONCHO™ VOTiVO™ In some aspects, the plant element may have a plant element coat comprising, e.g., Metalaxyl, and/or clothianidin, and/or Bacillus firmus-I-1582, upon which the present composition will be applied on top of, as a plant element overcoat. In some aspects, the taught microbial compositions are applied as a plant element overcoat to plant elements that have already been treated with ACCELERON™.

In some embodiments, the microorganism-treated plant elements have a microbial spore concentration, or microbial cell concentration, from about: 10\hat{ }2 to 10\hat{ }12, 10\hat{ }2 to 10\hat{ }11, 10\hat{ }2 to 10\hat{ }10, 10\hat{ }2 to 10\hat{ }9, 10\hat{ }2 to 10\hat{ }8, 10\hat{ }2 to 10\hat{ }7, 10\hat{ }2 to 10\hat{ }6, 10\hat{ }2 to 10\hat{ }5, 10\hat{ }2 to 10\hat{ }4, or 10\hat{ }2 to 10\hat{ }3 per plant element.

In some embodiments, the microorganism-treated plant elements have a microbial spore concentration, or microbial cell concentration, from about: 10\hat{ }3 to 10\hat{ }12, 10\hat{ }3 to 10\hat{ }11, 10\hat{ }3 to 10\hat{ }10, 10\hat{ }3 to 10\hat{ }9, 10\hat{ }3 to 10\hat{ }8, 10\hat{ }3 to 10\hat{ }7, 10\hat{ }3 to 10\hat{ }6, 10\hat{ }3 to 10\hat{ }5, or 10\hat{ }3 to 10\hat{ }4 per plant element.

In some embodiments, the microorganism-treated plant elements have a microbial spore concentration, or microbial cell concentration, from about: 10\hat{ }4 to 10\hat{ }12, 10\hat{ }4 to 10\hat{ }11, 10\hat{ }4 to 10\hat{ }10, 10\hat{ }4 to 10\hat{ }9, 10\hat{ }4 to 10\hat{ }8, 10\hat{ }4 to 10\hat{ }7, 10\hat{ }4 to 10\hat{ }6, or 10\hat{ }4 to 10\hat{ }5 per plant element.

In some embodiments, the microorganism-treated plant elements have a microbial spore concentration, or microbial cell concentration, from about: 10\hat{ }5 to 10\hat{ }12, 10\hat{ }5 to 10\hat{ }11, 10\hat{ }5 to 10\hat{ }10, 10\hat{ }5 to 10\hat{ }9, 10\hat{ }5 to 10\hat{ }8, 10\hat{ }5 to 10\hat{ }7, or 10\hat{ }5 to 10\hat{ }6 per plant element.

In some embodiments, the microorganism-treated plant elements have a microbial spore concentration, or microbial cell concentration, from about: 105 to 109 per plant element.

In some embodiments, the microorganism-treated plant elements have a microbial spore concentration, or microbial cell concentration, of at least about: 1×10\hat{ }3, or 1×10\hat{ }4, or 1×10\hat{ }5, or 1×10\hat{ }6, or 1×10\hat{ }7, or 1×10\hat{ }8, or 1×10\hat{ }9 per plant element.

In some embodiments, the amount of one or more of the microbes and/or compositions applied to the plant element depend on the final formulation, as well as size or type of the plant or plant element utilized. In some embodiments, one or more of the microbes are present in about 2% w/w to about 80% w/w of the entire formulation. In some embodiments, the one or more of the microbes employed in the compositions is about 5% w/w to about 65% w/w, or 10% w/w to about 60% w/w by weight of the entire formulation.

In some embodiments, the plant elements may also have more spores or microbial cells per plant element, such as, for example about 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, 10\hat{ }15, 10\hat{ }16, or 10\hat{ }17 spores or cells per plant element.

In some embodiments, the plant element coats of the present disclosure can be up to 10 um, 20 um, 30 um, 40 um, 50 um, 60 um, 70 um, 80 um, 90 um, 100 um, 110 um, 120 um, 130 um, 140 um, 150 um, 160 um, 170 um, 180 um, 190 um, 200 um, 210 um, 220 um, 230 um, 240 um, 250 um, 260 um, 270 um, 280 um, 290 um, 300 um, 310 um, 320 um, 330 um, 340 um, 350 um, 360 um, 370 um, 380 um, 390 um, 400 um, 410 um, 420 um, 430 um, 440 um, 450 um, 460 um, 470 um, 480 um, 490 um, 500 um, 510 um, 520 um, 530 um, 540 um, 550 um, 560 um, 570 um, 580 um, 590 um, 600 um, 610 um, 620 um, 630 um, 640 um, 650 um, 660 um, 670 um, 680 um, 690 um, 700 um, 710 um, 720 um, 730 um, 740 um, 750 um, 760 um, 770 um, 780 um, 790 um, 800 um, 810 um, 820 um, 830 um, 840 um, 850 um, 860 um, 870 um, 880 um, 890 um, 900 um, 910 um, 920 um, 930 um, 940 um, 950 um, 960 um, 970 um, 980 um, 990 um, 1000 um, 1010 um, 1020 um, 1030 um, 1040 um, 1050 um, 1060 um, 1070 um, 1080 um, 1090 um, 1100 um, 1110 um, 1120 um, 1130 um, 1140 um, 1150 um, 1160 um, 1170 um, 1180 um, 1190 um, 1200 um, 1210 um, 1220 um, 1230 um, 1240 um, 1250 um, 1260 um, 1270 um, 1280 um, 1290 um, 1300 um, 1310 um, 1320 um, 1330 um, 1340 um, 1350 um, 1360 um, 1370 um, 1380 um, 1390 um, 1400 um, 1410 um, 1420 um, 1430 um, 1440 um, 1450 um, 1460 um, 1470 um, 1480 um, 1490 um, 1500 um, 1510 um, 1520 um, 1530 um, 1540 um, 1550 um, 1560 um, 1570 um, 1580 um, 1590 um, 1600 um, 1610 um, 1620 um, 1630 um, 1640 um, 1650 um, 1660 um, 1670 um, 1680 um, 1690 um, 1700 um, 1710 um, 1720 um, 1730 um, 1740 um, 1750 um, 1760 um, 1770 um, 1780 um, 1790 um, 1800 um, 1810 um, 1820 um, 1830 um, 1840 um, 1850 um, 1860 um, 1870 um, 1880 um, 1890 um, 1900 um, 1910 um, 1920 um, 1930 um, 1940 um, 1950 um, 1960 um, 1970 um, 1980 um, 1990 um, 2000 um, 2010 um, 2020 um, 2030 um, 2040 um, 2050 um, 2060 um, 2070 um, 2080 um, 2090 um, 2100 um, 2110 um, 2120 um, 2130 um, 2140 um, 2150 um, 2160 um, 2170 um, 2180 um, 2190 um, 2200 um, 2210 um, 2220 um, 2230 um, 2240 um, 2250 um, 2260 um, 2270 um, 2280 um, 2290 um, 2300 um, 2310 um, 2320 um, 2330 um, 2340 um, 2350 um, 2360 um, 2370 um, 2380 um, 2390 um, 2400 um, 2410 um, 2420 um, 2430 um, 2440 um, 2450 um, 2460 um, 2470 um, 2480 um, 2490 um, 2500 um, 2510 um, 2520 um, 2530 um, 2540 um, 2550 um, 2560 um, 2570 um, 2580 um, 2590 um, 2600 um, 2610 um, 2620 um, 2630 um, 2640 um, 2650 um, 2660 um, 2670 um, 2680 um, 2690 um, 2700 um, 2710 um, 2720 um, 2730 um, 2740 um, 2750 um, 2760 um, 2770 um, 2780 um, 2790 um, 2800 um, 2810 um, 2820 um, 2830 um, 2840 um, 2850 um, 2860 um, 2870 um, 2880 um, 2890 um, 2900 um, 2910 um, 2920 um, 2930 um, 2940 um, 2950 um, 2960 um, 2970 um, 2980 um, 2990 um, or 3000 um thick.

In some embodiments, the plant element coats of the present disclosure can be 0.5 mm, 1 mm, 1.5 mm, 2 mm, 2.5 mm, 3 mm, 3.5 mm, 4 mm, 4.5 mm, or 5 mm thick.

In some embodiments, the plant element coats of the present disclosure can be at least 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, or 50% of the uncoated plant element weight.

In some embodiments, the microbes and/or compositions can be coated freely onto the plant elements or they can be formulated in a liquid or solid composition before being coated onto the plant elements. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, it is contemplated that the solid or liquid microbial compositions of the present disclosure further contain functional agents e.g., activated carbon, nutrients (fertilizers), and other agents capable of improving the germination and quality of the products or a combination thereof.

Plant element coating methods and compositions that are known in the art can be particularly useful when they are modified by the addition of one of the embodiments of the present disclosure. Such coating methods and apparatus for their application are disclosed in, for example: U.S. Pat. Nos. 5,916,029; 5,918,413; 5,554,445; 5,389,399; 4,759,945; 4,465,017, and U.S. patent application Ser. No. 13/260,310, each of which is incorporated by reference herein.

Plant element coating compositions are disclosed in, for example: U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432, each of which is incorporated by reference herein.

In some embodiments, a variety of additives can be added to the plant element treatment formulations comprising the inventive compositions. Binders can be added and include those composed of an adhesive polymer that can be natural or synthetic without phytotoxic effect on the plant element to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorants may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, molybdenum and zinc.

A polymer or other dust control agent can be applied to retain the treatment on the plant element surface.

In some specific embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches. More examples can be found in, for example, U.S. Pat. No. 7,213,367, incorporated herein by reference.

Various additives, such as adherents, dispersants, surfactants, and nutrient and buffer ingredients, can also be included in the plant element treatment formulation. Other conventional plant element treatment additives include, but are not limited to: coating agents, wetting agents, buffering agents, and polysaccharides. At least one agriculturally acceptable carrier can be added to the plant element treatment formulation such as water, solids, or dry powders. The dry powders can be derived from a variety of materials such as calcium carbonate, gypsum, vermiculite, talc, humus, activated charcoal, and various phosphorous compounds.

In some embodiments, the plant element coating composition can comprise at least one filler, which is an organic or inorganic, natural or synthetic component with which the active components are combined to facilitate its application onto the plant element. In aspects, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

In some embodiments, the plant element treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the plant element, or alternatively may be added as part of the plant element coating composition of the disclosure.

In some embodiments, the formulation that is used to treat the plant element in the present disclosure can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation can be about 0.5% to about 99% by weight (w/w), or 5-40%, or as otherwise formulated by those skilled in the art.

As mentioned above, other conventional inactive or inert ingredients can be incorporated into the formulation. Such inert ingredients include, but are not limited to: conventional sticking agents; dispersing agents such as methylcellulose, for example, serve as combined dispersant/sticking agents for use in plant element treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the present disclosure can be found in McCutcheon's, vol. 1, “Emulsifiers and Detergents,” MC Publishing Company, Glen Rock, N.J., U.S.A., 1996, incorporated by reference herein.

The plant element coating formulations of the present disclosure can be applied to plant elements by a variety of methods, including, but not limited to: mixing in a container (e.g., a bottle or bag), mechanical application, tumbling, spraying, and immersion. A variety of active or inert material can be used for contacting plant elements with microbial compositions according to the present disclosure.

In some embodiments, the amount of the microbes or agricultural composition that is used for the treatment of the plant element will vary depending upon the type of plant element and the type of active ingredients, but the treatment will comprise contacting the plant elements with an agriculturally effective amount of the inventive composition.

As discussed above, an effective amount means that amount of the inventive composition that is sufficient to affect beneficial or desired results. An effective amount can be administered in one or more administrations.

In some embodiments, in addition to the coating layer, the plant element may be treated with one or more of the following ingredients: other pesticides including fungicides and herbicides; herbicidal safeners; fertilizers and/or biocontrol agents. These ingredients may be added as a separate layer or alternatively may be added in the coating layer.

In some embodiments, the plant element coating formulations of the present disclosure may be applied to the plant elements using a variety of techniques and machines, such as fluidized bed techniques, the roller mill method, rotostatic plant element treaters, and drum coaters. Other methods, such as spouted beds may also be useful. The plant elements may be pre-sized before coating. After coating, the plant elements are typically dried and then transferred to a sizing machine for sizing. Such procedures are known in the art.

In some embodiments, the microorganism-treated plant elements may also be enveloped with a film overcoating to protect the coating. Such overcoatings are known in the art and may be applied using fluidized bed and drum film coating techniques.

In other embodiments of the present disclosure, compositions according to the present disclosure can be introduced onto a plant element by use of solid matrix priming. For example, a quantity of an inventive composition can be mixed with a solid matrix material and then the plant element can be placed into contact with the solid matrix material for a period to allow the composition to be introduced to the plant element. The plant element can then optionally be separated from the solid matrix material and stored or used, or the mixture of solid matrix material plus plant element can be stored or planted directly. Solid matrix materials which are useful in the present disclosure include polyacrylamide, starch, clay, silica, alumina, soil, sand, polyurea, polyacrylate, or any other material capable of absorbing or adsorbing the inventive composition for a time and releasing that composition into or onto the plant element. It is useful to make sure that the inventive composition and the solid matrix material are compatible with each other. For example, the solid matrix material should be chosen so that it can release the composition at a reasonable rate, for example over a period of minutes, hours, or days.

In some embodiments, the present disclosure teaches that the individual microbes, or microbial consortia, or microbial communities, developed according to the disclosed methods can be combined with any plant biostimulant.

In some embodiments, the present disclosure teaches compositions comprising one or more commercially available biostimulants, including but not limited to: Vitazyme®, Diehard™ Biorush®, Diehard™ Biorush® Fe, Diehard™ Soluble Kelp, Diehard™ Humate SP, Phocon®, Foliar Plus™, Plant Plus™, Accomplish LM®, Titan®, Soil Builder™, Nutri Life, Soil Solution™, Seed Coat™, PercPlus™, Plant Power®, CropKarb®, Thrust™, Fast2Grow®, Baccarat®, and Potente® among others.

In some embodiments, when the microbe or microbial consortia identified according to the taught methods is combined with an active chemical agent one witnesses an additive effect on a plant phenotypic trait of interest. In other embodiments, when the microbe or microbial consortia identified according to the taught methods is combined with an active chemical agent one witness a synergistic effect on a plant phenotypic trait of interest.

In some embodiments, when the microbe or microbial consortia identified according to the taught methods is combined with a fertilizer one witnesses an additive effect on a plant phenotypic trait of interest. In other embodiments, when the microbe or microbial consortia identified according to the taught methods is combined with a fertilizer one witness a synergistic effect on a plant phenotypic trait of interest.

In some embodiments, when the microbe or microbial consortia identified according to the taught methods is combined with a plant growth regulator, one witnesses an additive effect on a plant phenotypic trait of interest. In some embodiments, when the microbe or microbial consortia identified according to the taught methods is combined with a plant growth regulator, one witnesses a synergistic effect. In some aspects, the microbes of the present disclosure are combined with Ascend® and a synergistic effect is observed for one or more phenotypic traits of interest.

In some embodiments, when the microbe or microbial consortia identified according to the taught methods is combined with a biostimulant, one witnesses an additive effect on a plant phenotypic trait of interest. In some embodiments, when the microbe or microbial consortia identified according to the taught methods is combined with a biostimulant, one witnesses a synergistic effect.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agricultural active compounds and also agricultural auxiliary compounds.

In other embodiments, when the microbe or microbial consortia identified according to the taught methods is combined with a fertilizer one witnesses a synergistic effect.

Furthermore, in certain embodiments, the disclosure utilizes synergistic interactions to define microbial consortia. That is, in certain aspects, the disclosure combines together certain isolated microbial species, which act synergistically, into consortia that impart a beneficial trait upon a plant, or which are correlated with increasing a beneficial plant trait.

The compositions developed according to the disclosure can be formulated with certain auxiliaries, in order to improve the activity of a known active agricultural compound. This has the advantage that the amounts of active ingredient in the formulation may be reduced while maintaining the efficacy of the active compound, thus allowing costs to be kept as low as possible and any official regulations to be followed. In individual cases, it may also possible to widen the spectrum of action of the active compound since plants, where the treatment with a particular active ingredient without addition was insufficiently successful, can indeed be treated successfully by the addition of certain auxiliaries along with the disclosed microbial isolates and consortia. Moreover, the performance of the active may be increased in individual cases by a suitable formulation when the environmental conditions are not favorable.

Such auxiliaries that can be used in an agricultural composition can be an adjuvant. Frequently, adjuvants take the form of surface-active or salt-like compounds. Depending on their mode of action, they can roughly be classified as modifiers, activators, fertilizers, pH buffers, and the like. Modifiers affect the wetting, sticking, and spreading properties of a formulation. Activators break up the waxy cuticle of the plant and improve the penetration of the active ingredient into the cuticle, both short-term (over minutes) and long-term (over hours). Fertilizers such as ammonium sulfate, ammonium nitrate or urea improve the absorption and solubility of the active ingredient and may reduce the antagonistic behavior of active ingredients. pH buffers are conventionally used for bringing the formulation to an optimal pH.

For further embodiments of compositions of the present disclosure, See “Chemistry and Technology of Agrochemical Formulations,” edited by D. A. Knowles, copyright 1998 by Kluwer Academic Publishers, hereby incorporated by reference.

### Plants and Agronomic Benefits

A wide variety of plants, including those cultivated in agriculture, are capable of receiving benefit from the application of microbes, such as those described herein, including single microbes, consortia, and/or compositions produced therefrom, or comprising any of the preceding. Any number of a variety of different plants, including mosses and lichens and algae, may be used in the methods of the disclosure. In embodiments, the plants have economic, social, or environmental value. For example, the plants may include those used as: food crops, fiber crops, oil crops, in the forestry industry, in the pulp and paper industry, as a feedstock for biofuel production, and as ornamental plants.

In other embodiments, the plants may be economically, socially, or environmentally undesirable, such as weeds. The following is a list of non-limiting examples of the types of plants the methods of the disclosure may be applied to plant parts or plants that include the following.

### Food Crops

Cereals e.g maize, rice, wheat, barley, sorghum, millet, oats, rye, triticale, and buckwheat;

Leafy vegetables e.g., brassicaceous plants such as cabbages, broccoli, bok choy, rocket; salad greens such as spinach, cress, and lettuce;

Fruiting and flowering vegetables e.g., avocado, sweet corn, artichokes; cucurbits e.g., squash, cucumbers, melons, courgettes, pumpkins; solanaceous vegetables/fruits e.g., tomatoes, eggplant, and capsicums;

Podded vegetables e.g., groundnuts, peanuts, peas, soybeans, beans, lentils, chickpea, okra;

Bulbed and stem vegetables e.g., asparagus, celery, Allium crops e.g garlic, onions, and leeks;

Roots and tuberous vegetables e.g., carrots, beet, bamboo shoots, cassava, yams, ginger, Jerusalem artichoke, parsnips, radishes, potatoes, sweet potatoes, taro, turnip, and wasabi;

Sugar crops including sugar beet (Beta vulgaris), sugar cane (Saccharum officinarum);

Crops grown for the production of non-alcoholic beverages and stimulants e.g., coffee, black, herbal, and green teas, cocoa, marijuana, and tobacco;

Fruit crops such as true berry fruits (e.g., kiwifruit, grape, currants, gooseberry, guava, feijoa, pomegranate), citrus fruits (e.g., oranges, lemons, limes, grapefruit), epigynous fruits (e.g., bananas, cranberries, blueberries), aggregate fruit (blackberry, raspberry, boysenberry), multiple fruits (e.g., pineapple, fig), stone fruit crops (e.g., apricot, peach, cherry, plum), pip-fruit (e.g., apples, pears) and others such as strawberries, sunflower seeds;

Culinary and medicinal herbs e.g., rosemary, basil, bay laurel, coriander, mint, dill, Hypericum, foxglove, aloe vera, rosehips, and cannabis;

Crop plants producing spices e.g., black pepper, cumin cinnamon, nutmeg, ginger, cloves, saffron, cardamom, mace, paprika, masalas, star anise;

Crops grown for the production of nuts e.g., almonds and walnuts, Brazil nut, cashew nuts, coconuts, chestnut, macadamia nut, pistachio nuts; peanuts, pecan nuts;

Crops grown for production of beers, wines and other alcoholic beverages e.g grapes, and hops;

Oilseed crops e.g., soybean, peanuts, cotton, olives, sunflower, sesame, lupin species and brassicaeous crops (e.g., canola/oilseed rape); and, edible fungi e.g., white mushrooms, Shiitake and oyster mushrooms;

### Plants Used in Pastoral Agriculture

Legumes: Trifolium species, Medicago species, and Lotus species; White clover (T. repens); Red clover (T. pratense); Caucasian clover (T. ambigum); subterranean clover (T. subterraneum); Alfalfa/Lucerne (Medicago sativum); annual medics; barrel medic; black medic; Sainfoin (Onobrychis viciifolia); Birdsfoot trefoil (Lotus corniculatus); Greater Birdsfoot trefoil (Lotus pedunculatus);

Seed legumes/pulses including Peas (Pisum sativum), Common bean (Phaseolus vulgaris), Broad beans (Vicia faba), Mung bean (Vigna radiata), Cowpea (Vigna unguiculata), Chick pea (Cicer arietum), Lupins (Lupinus species); Cereals including Maize/corn (Zea mays), Sorghum (Sorghum spp.), Millet (Panicum miliaceum, P. sumatrense), Rice (Oryza sativa indica, Oryza sativa japonica), Wheat (Triticum aestivum), Barley (Hordeum vulgare), Rye (Secale cereale), Triticale (Triticum X Secale), Oats (Avena sativa);

Forage and Amenity grasses: Temperate grasses such as Lolium species; Festuca species; Agrostis spp., Perennial ryegrass (Lolium perenne); hybrid ryegrass (Lolium hybridum); annual ryegrass (Lolium multiflorum), tall fescue (Festuca arundinacea); meadow fescue (Festuca pratensis); red fescue (Festuca rubra); Festuca ovina; Festuloliums (Lolium X Festuca crosses); Cocksfoot (Dactylis glomerata); Kentucky bluegrass Poa pratensis; Poa palustris; Poa nemoralis; Poa trivialis; Poa compresa; Bromus species; Phalaris (Phleum species); Arrhenatherum elatius; Agropyron species; Avena strigosa; Setaria italic;

Tropical grasses such as: Phalaris species; Brachiaria species; Eragrostis species; Panicum species; Bahai grass (Paspalum notatum); Brachypodium species; and grasses used for biofuel production such as Switchgrass (Panicum virgatum) and Miscanthus species.

### Fiber Crops

Cotton, hemp, jute, coconut, sisal, flax (Linum spp.), New Zealand flax (Phormium spp.); plantation and natural forest species harvested for paper and engineered wood fiber products such as coniferous and broadleafed forest species.

### Tree and Shrub Species Used in Plantation Forestry and Bio-Fuel Crops

Pine (Pinus species); Fir (Pseudotsuga species); Spruce (Picea species); Cypress (Cupressus species); Wattle (Acacia species); Alder (Alnus species); Oak species (Quercus species); Redwood (Sequoiadendron species); willow (Salix species); birch (Betula species); Cedar (Cedurus species); Ash (Fraxinus species); Larch (Larix species); Eucalyptus species; Bamboo (Bambuseae species) and Poplars (Populus species).

### Plants Grown for Conversion to Energy, Biofuels or Industrial Products by Extractive, Biological, Physical or Biochemical Treatment

Oil-producing plants such as oil palm, jatropha, soybean, cotton, linseed; Latex-producing plants such as the Para Rubber tree, Hevea brasiliensis and the Panama Rubber Tree Castilla elastica; plants used as direct or indirect feedstocks for the production of biofuels i.e., after chemical, physical (e.g., thermal or catalytic) or biochemical (e.g., enzymatic pre-treatment) or biological (e.g., microbial fermentation) transformation during the production of biofuels, industrial solvents or chemical products e.g., ethanol or butanol, propane dials, or other fuel or industrial material including sugar crops (e.g., beet, sugar cane), starch producing crops (e.g., C3 and C4 cereal crops and tuberous crops), cellulosic crops such as forest trees (e.g., Pines, Eucalypts) and Graminaceous and Poaceous plants such as bamboo, switch grass, miscanthus; crops used in energy, biofuel or industrial chemical production via gasification and/or microbial or catalytic conversion of the gas to biofuels or other industrial raw materials such as solvents or plastics, with or without the production of biochar (e.g., biomass crops such as coniferous, eucalypt, tropical or broadleaf forest trees, graminaceous and poaceous crops such as bamboo, switch grass, miscanthus, sugar cane, or hemp or softwoods such as poplars, willows; and, biomass crops used in the production of biochar.

### Crops Producing Natural Products Useful for the Pharmaceutical, Agricultural Nutraceutical and Cosmeceutical Industries

Crops producing pharmaceutical precursors or compounds or nutraceutical and cosmeceutical compounds and materials for example, star anise (shikimic acid), Japanese knotweed (resveratrol), kiwifruit (soluble fiber, proteolytic enzymes).

Floricultural, Ornamental and Amenity Plants Grown for their Aesthetic or Environmental Properties

Flowers such as roses, tulips, chrysanthemums.

Ornamental shrubs such as Buxus, Hebe, Rosa, Rhododendron, Hedera.

Amenity plants such as Platanus, Choisya, Escallonia, Euphorbia, Carex.

Mosses such as sphagnum moss.

### Plants Grown for Bioremediation

In certain aspects, the microbes of the present disclosure are applied to hybrid plants to increase beneficial traits of said hybrids. In other aspects, the microbes of the present disclosure are applied to genetically modified plants to increase beneficial traits of said GM plants. The microbes taught herein are able to be applied to hybrids and GM plants and thus maximize the elite genetics and trait technologies of these plants.

It should be appreciated that a plant may be provided in the form of a seed, seedling, cutting, propagule, or any other plant material or tissue capable of growing. In one embodiment the seed may be surface-sterilized with a material such as sodium hypochlorite or mercuric chloride to remove surface-contaminating microorganisms. In one embodiment, the propagule is grown in axenic culture before being placed in the plant growth medium, for example as sterile plantlets in tissue culture.

### Hybrid and Genetically-Modified (GM) Plant Improvement

In certain aspects, the microbes of the present disclosure are applied to hybrid plants to increase beneficial traits of said hybrids. In other aspects, the microbes of the present disclosure are applied to genetically modified plants to increase beneficial traits of said GM plants. The microbes taught herein are able to be applied to hybrids and GM plants and thus maximize the elite genetics and trait technologies of these plants.

It should be appreciated that a plant may be provided in the form of a seed, seedling, cutting, propagule, or any other plant material or tissue capable of growing. In one embodiment the seed may be surface-sterilized with a material such as sodium hypochlorite or mercuric chloride to remove surface-contaminating microorganisms. In one embodiment, the propagule is grown in axenic culture before being placed in the plant growth medium, for example as sterile plantlets in tissue culture.

### Methods of Application

The microorganisms may be applied to a plant, seedling, cutting, propagule, or the like and/or the growth medium containing said plant, using any appropriate technique known in the art.

However, by way of example, an isolated microbe, consortia, or composition comprising the same, and/or a composition produced therefrom, may be applied to a plant, seedling, cutting, propagule, or the like, by spraying, coating, dusting, or any other method known in the art.

In another embodiment, the isolated microbe, consortia, or composition comprising the same may be applied directly to a plant seed prior to sowing.

In another embodiment, the isolated microbe, consortia, composition produced therefrom, or composition comprising the same may applied directly to a plant seed, as a seed coating.

In one embodiment of the present disclosure, the isolated microbe, consortia, or composition comprising the same is supplied in the form of granules, or plug, or soil drench that is applied to the plant growth media.

In other embodiments, the isolated microbe, consortia, or composition comprising the same are supplied in the form of a foliar application, such as a foliar spray or liquid composition. The foliar spray or liquid application may be applied to a growing plant or to a growth media, e.g., soil.

In some embodiments, the isolated microbe, consortia, or composition comprising the same are supplied in a form selected from: a soil drench, a foliar spray, a dip treatment, an in-furrow treatment, a soil amendment, granules, a broadcast treatment, a post-harvest disease control treatment, or a seed treatment. In some embodiments, the compositions may be applied alone in or in rotation spray programs.

In some embodiments, the isolated microbe, consortia, or composition comprising the same may be compatible with tank mixing. In some embodiments, the compositions may be compatible with tank mixing with other agricultural products. In some embodiments, the compositions may be compatible with equipment used for ground, aerial, and irrigation applications.

In another embodiment, the isolated microbe, consortia, or composition comprising the same may be formulated into granules and applied alongside seeds during planting. Or the granules may be applied after planting. Or the granules may be applied before planting.

In some embodiments, the isolated microbe, consortia, or composition comprising the same are administered to a plant or growth media as a topical application and/or drench application to improve crop growth, yield, and quality. The topical application may be via utilization of a dry mix or powder or dusting composition or may be a liquid based formulation.

In embodiments, the isolated microbe, consortia, or composition comprising the same can be formulated as: (1) solutions; (2) wettable powders; (3) dusting powders; (4) soluble powders; (5) emulsions or suspension concentrates; (6) seed dressings or coatings, (7) tablets; (8) water-dispersible granules; (9) water soluble granules (slow or fast release); (10) microencapsulated granules or suspensions; (11) as irrigation components, and (12) a component of fertilizers, pesticides, and other compatible amendments, among others. In certain aspects, the compositions may be diluted in an aqueous medium prior to conventional spray application. The compositions of the present disclosure can be applied to the soil, plant, seed, rhizosphere, rhizosheath, rhizoplane, or other area to which it would be beneficial to apply the microbial compositions. Further still, ballistic methods can be utilized as a means for introducing endophytic microbes.

In aspects, the compositions are applied to the foliage of plants. The compositions may be applied to the foliage of plants in the form of an emulsion or suspension concentrate, liquid solution, or foliar spray. The application of the compositions may occur in a laboratory, growth chamber, greenhouse, or in the field.

In another embodiment, microorganisms may be inoculated into a plant by cutting the roots or stems and exposing the plant surface to the microorganisms by spraying, dipping, or otherwise applying a liquid microbial suspension, or gel, or powder.

In another embodiment, the microorganisms may be injected directly into foliar or root tissue, or otherwise inoculated directly into or onto a foliar or root cut, or else into an excised embryo, or radicle, or coleoptile. These inoculated plants may then be further exposed to a growth media containing further microorganisms; however, this is not necessary.

In other embodiments, particularly where the microorganisms are unculturable, the microorganisms may be transferred to a plant by any one or a combination of grafting, insertion of explants, aspiration, electroporation, wounding, root pruning, induction of stomatal opening, or any physical, chemical or biological treatment that provides the opportunity for microbes to enter plant cells or the intercellular space. Persons of skill in the art may readily appreciate a number of alternative techniques that may be used.

In one embodiment, the microorganisms infiltrate parts of the plant such as the roots, stems, leaves and/or reproductive plant parts (become endophytic), and/or grow upon the surface of roots, stems, leaves and/or reproductive plant parts (become epiphytic) and/or grow in the plant rhizosphere. In one embodiment, the microorganisms form a symbiotic relationship with the plant.

In some embodiments, the present disclosure also concerns the discovery that treating seeds before they are sown or planted with a composition of the present disclosure can enhance a desired plant trait, e.g. plant growth, plant health, and/or plant resistance to pests.

Thus, in some embodiments, the present disclosure teaches the use of the compositions of the disclosure as seed treatments. The seed treatment can be a seed coating applied directly to an untreated and “naked” seed. However, the seed treatment can be a seed overcoat that is applied to a seed that has already been coated with one or more previous seed coatings or seed treatments. The previous seed treatments may include one or more active compounds, either chemical or biological, and one or more inert ingredients.

The term “seed treatment” generally refers to application of a material to a seed prior to or during the time it is planted in soil. Seed treatment with the compositions of the present disclosure, has the advantages of delivering the treatments to the locus at which the seeds are planted shortly before germination of the seed and emergence of a seedling.

In other embodiments, the present disclosure also teaches that the use of seed treatments minimizes the amount of the composition of the disclosure that is required to successfully treat the plants, and further limits the amount of contact of workers with the compositions compared to application techniques such as spraying over soil or over emerging seedlings.

Moreover, in some embodiments, the present disclosure teaches that the compositions disclosed herein are important for enhancing the early stages of plant life (e.g., within the first thirty days following emergence of the seedling). Thus, in some embodiments, delivery of the compositions of the present disclosure as a seed treatment places the composition at the locus of action at a critical time for its activity.

In some embodiments, the compositions of the present disclosure are formulated as a seed treatment. In some embodiments, it is contemplated that the seeds can be substantially uniformly coated with one or more layers of the compositions disclosed herein, using conventional methods of mixing, spraying, or a combination thereof through the use of treatment application equipment that is specifically designed and manufactured to accurately, safely, and efficiently apply seed treatment products to seeds.

Such equipment uses various types of coating technology such as rotary coaters, drum coaters, fluidized bed techniques, spouted beds, rotary mists, or a combination thereof. Liquid seed treatments such as those of the present disclosure can be applied via either a spinning “atomizer” disk or a spray nozzle, which evenly distributes the seed treatment onto the seed as it moves though the spray pattern. In aspects, the seed is then mixed or tumbled for an additional period of time to achieve additional treatment distribution and drying.

The seeds can be primed or unprimed before coating with the microbial compositions to increase the uniformity of germination and emergence. In an alternative embodiment, a dry powder formulation can be metered onto the moving seed and allowed to mix until completely distributed.

In some embodiments, the seeds have at least part of the surface area coated with a composition of the disclosure, according to the methods disclosed herein. In some embodiments, a seed coat including the composition is applied directly to a naked seed. In some embodiments, a seed overcoat including the composition is applied to a seed that already has a seed coat applied thereon. In some aspects, the seed may have a seed coat that includes, e.g. clothianidin and/or Bacillus firmus-I-1582, upon which the present composition will be applied on top of, as a seed overcoat. In some aspects, the taught microbial compositions are applied as a seed overcoat to seeds that have already been treated with PONCHO™ VOTiVO™. In some aspects, the seed may have a seed coat that includes, e.g. Metalaxyl, and/or clothianidin, and/or Bacillus firmus-I-1582, upon which the present composition will be applied on top of, as a seed overcoat. In some aspects, the taught microbial compositions are applied as a seed overcoat to seeds that have already been treated with ACCELERON™. In some embodiments, the composition-treated seeds have a microbial spore concentration, or microbial cell concentration, from about: 10\hat{ }2 to 10\hat{ }12, 10\hat{ }2 to 10\hat{ }11, 10\hat{ }2 to 10\hat{ }10, 10\hat{ }2 to 10\hat{ }9, 10\hat{ }2 to 10\hat{ }8, 10\hat{ }2 to 10\hat{ }7, 10\hat{ }2 to 10\hat{ }6, 10\hat{ }2 to 10\hat{ }5, 10\hat{ }2 to 10\hat{ }4, or 10\hat{ }2 to 10\hat{ }3 per seed, provided that the composition includes a microorganism of the present disclosure.

In some embodiments, the composition-treated seeds have a microbial spore concentration, or microbial cell concentration, from about: 10\hat{ }3 to 10\hat{ }12, 10\hat{ }3 to 10\hat{ }11, 10\hat{ }3 to 10\hat{ }10, 10\hat{ }3 to 10\hat{ }9, 10\hat{ }3 to 10\hat{ }8, 10\hat{ }3 to 10\hat{ }7, 10\hat{ }3 to 10\hat{ }6, 10\hat{ }3 to 10\hat{ }5, or 10\hat{ }3 to 10\hat{ }4 per seed, provided that the composition includes a microorganism of the present disclosure.

In some embodiments, the composition-treated seeds have a microbial spore concentration, or microbial cell concentration, from about: 10\hat{ }4 to 10\hat{ }12, 10\hat{ }4 to 10\hat{ }11, 10\hat{ }4 to 10\hat{ }10, 10\hat{ }4 to 10\hat{ }9, 10\hat{ }4 to 10\hat{ }8, 10\hat{ }4 to 10\hat{ }7, 10\hat{ }4 to 10\hat{ }6, or 10\hat{ }4 to 10\hat{ }5 per seed, provided that the composition includes a microorganism of the present disclosure.

In some embodiments, the composition-treated seeds have a microbial spore concentration, or microbial cell concentration, from about: 10\hat{ }5 to 10\hat{ }12, 10\hat{ }5 to 10\hat{ }11, 10\hat{ }5 to 10\hat{ }10, 10\hat{ }5 to 10\hat{ }9, 10\hat{ }5 to 10\hat{ }8, 10\hat{ }5 to 10\hat{ }7, or 10\hat{ }5 to 10\hat{ }6 per seed, provided that the composition includes a microorganism of the present disclosure.

In some embodiments, the composition-treated seeds have a microbial spore concentration, or microbial cell concentration, from about: 10\hat{ }5 to 10\hat{ }9 per seed.

In some embodiments, the composition-treated seeds have a microbial spore concentration, or microbial cell concentration, of at least about: 1×10\hat{ }3, or 1×10\hat{ }4, or 1×10\hat{ }5, or 1×10\hat{ }6, or 1×10\hat{ }7, or 1×10\hat{ }8, or 1×10\hat{ }9 per seed, provided that the composition includes a microorganism of the present disclosure.

In some embodiments, the amount of the composition of the disclosure applied to the seed depends on the final formulation, as well as size or type of the plant or seed utilized. In some embodiments, one or more of the microbes of the disclosure are present in about 2% w/w/to about 80% w/w of the entire formulation. In some embodiments, the one or more of the microbes employed in the compositions of the disclosure is about 5% w/w to about 65% w/w, or 10% w/w to about 60% w/w by weight of the entire formulation.

In some embodiments, the seed coats of the present disclosure can be up to 10 um, 20 um, 30 um, 25 40 um, 50 um, 60 um, 70 um, 80 um, 90 um, 100 um, 110 um, 120 um, 130 um, 140 um, 150 um, 160 um, 26 170 um, 180 um, 190 um, 200 um, 210 um, 220 um, 230 um, 240 um, 250 um, 260 um, 270 um, 280 um, 27 290 um, 300 um, 310 um, 320 um, 330 um, 340 um, 350 um, 360 um, 370 um, 380 um, 390 um, 400 um, 28 410 um, 420 um, 430 um, 440 um, 450 um, 460 um, 470 um, 480 um, 490 um, 500 um, 510 um, 520 um, 29 530 um, 540 um, 550 um, 560 um, 570 um, 580 um, 590 um, 600 um, 610 um, 620 um, 630 um, 640 um, 30 650 um, 660 um, 670 um, 680 um, 690 um, 700 um, 710 um, 720 um, 730 um, 740 um, 750 um, 760 um, 31 770 um, 780 um, 790 um, 800 um, 810 um, 820 um, 830 um, 840 um, 850 um, 860 um, 870 um, 880 um, 32 890 um, 900 um, 910 um, 920 um, 930 um, 940 um, 950 um, 960 um, 970 um, 980 um, 990 um, 1000 um, 33 1010 um, 1020 um, 1030 um, 1040 um, 1050 um, 1060 um, 1070 um, 1080 um, 1090 um, 1100 um, 1110 um, 34 1120 um, 1130 um, 1140 um, 1150 um, 1160 um, 1170 um, 1180 um, 1190 um, 1200 um, 1210 um, 1220 um, 1230 um, 1240 um, 1250 um, 1260 um, 1270 um, 1280 um, 1290 um, 1300 um, 1310 um, 1320 um, 1330 um, 2 1340 um, 1350 um, 1360 um, 1370 um, 1380 um, 1390 um, 1400 um, 1410 um, 1420 um, 1430 um, 1440 um, 1450 um, 1460 um, 1470 um, 1480 um, 1490 um, 1500 um, 1510 um, 1520 um, 1530 um, 1540 um, 1550 um, 1560 um, 1570 um, 1580 um, 1590 um, 1600 um, 1610 um, 1620 um, 1630 um, 1640 um, 1650 um, 1660 um, 1670 um, 1680 um, 1690 um, 1700 um, 1710 um, 1720 um, 1730 um, 1740 um, 1750 um, 1760 um, 1770 um, 1780 um, 1790 um, 1800 um, 1810 um, 1820 um, 1830 um, 1840 um, 1850 um, 1860 um, 1870 um, 1880 um, 1890 um, 1900 um, 1910 um, 1920 um, 1930 um, 1940 um, 1950 um, 1960 um, 1970 um, 1980 um, 1990 um, 2000 um, 2010 um, 2020 um, 2030 um, 2040 um, 2050 um, 2060 um, 2070 um, 2080 um, 2090 um, 2100 um, 2110 um, 2120 um, 2130 um, 2140 um, 2150 um, 2160 um, 2170 um, 2180 um, 2190 um, 2200 um, 2210 um, 2220 um, 2230 um, 2240 um, 2250 um, 2260 um, 2270 um, 2280 um, 2290 um, 2300 um, 2310 um, 2320 um, 2330 um, 2340 um, 2350 um, 2360 um, 2370 um, 2380 um, 2390 um, 2400 um, 2410 um, 2420 um, 2430 um, 2440 um, 2450 um, 2460 um, 2470 um, 2480 um, 2490 um, 2500 um, 2510 um, 2520 um, 2530 um, 2540 um, 2550 um, 2560 um, 2570 um, 2580 um, 2590 um, 2600 um, 2610 um, 2620 um, 2630 um, 2640 um, 2650 um, 2660 um, 2670 um, 2680 um, 2690 um, 2700 um, 2710 um, 2720 um, 2730 um, 2740 um, 2750 um, 2760 um, 2770 um, 2780 um, 2790 um, 2800 um, 2810 um, 2820 um, 2830 um, 2840 um, 2850 um, 2860 um, 2870 um, 2880 um, 2890 um, 2900 um, 2910 um, 2920 um, 2930 um, 2940 um, 2950 um, 2960 um, 2970 um, 2980 um, 2990 um, or 3000 um thick.

In some embodiments, the seed coats of the present disclosure can be 0.5 mm, 1 mm, 1.5 mm, 2 mm, 2.5 mm, 3 mm, 3.5 mm, 4 mm, 4.5 mm, or 5 mm thick.

In some embodiments, the seed coats of the present disclosure can be at least 0.5%, 1%, 1.5%, 2%, 21 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 22 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 24 27.5%, 28%, 28.5%, 29%, 29.5%, 30%, 30.5%, 31%, 31.5%, 32%, 32.5%, 33%, 33.5%, 34%, 34.5%, 35%, 25 35.5%, 36%, 36.5%, 37%, 37.5%, 38%, 38.5%, 39%, 39.5%, 40%, 40.5%, 41%, 41.5%, 42%, 42.5%, 43%, 26 43.5%, 44%, 44.5%, 45%, 45.5%, 46%, 46.5%, 47%, 47.5%, 48%, 48.5%, 49%, 49.5%, or 50% of the uncoated seed weight.

In some embodiments, the microbial spores and/or cells can be coated freely onto the seeds or they can be formulated in a liquid or solid composition before being coated onto the seeds. For example, a solid composition including the microorganisms can be prepared by mixing a solid carrier with a suspension of the spores until the solid carriers are impregnated with the spore or cell suspension. This mixture can then be dried to obtain the desired particles.

In some other embodiments, it is contemplated that the solid or liquid compositions of the present disclosure further contain functional agents e.g., activated carbon, nutrients (fertilizers), and other agents capable of improving the germination and quality of the products or a combination thereof.

Seed coating methods and compositions that are known in the art can be particularly useful when they are modified by the addition of one of the embodiments of the present disclosure. Such coating methods and apparatus for their application are disclosed in, for example: U.S. Pat. Nos. 5,916,029; 5,918,413; 5,554,445; 5,389,399; 4,759,945; 4,465,017, and U.S. Pat. App. Publication No. US20120015806A1 published 19 Jan. 2012; each of which is incorporated by reference herein.

Seed coating compositions are disclosed in, for example: U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432, each of which is incorporated by reference herein.

In some embodiments, a variety of additives can be added to the seed treatment formulations that include the inventive compositions. Binders can be added and include those composed of an adhesive polymer that can be natural or synthetic without phytotoxic effect on the seed to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses, and carboxymethylcellulose; polyvinylpyrolidones; polysaccharides, including starch, modified starch, dextrins, maltodextrins, alginate, and chitosans; fats; oils; proteins, including gelatin and zeins; gum arabics; shellacs; vinylidene chloride, and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorants may be employed, including organic chromophores classified as nitroso; nitro; azo, including monoazo, bisazo, and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, molybdenum, and zinc.

A polymer or other dust control agent can be applied to retain the treatment on the seed surface.

In some specific embodiments, in addition to the microbial cells or spores, the coating can further include a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches. More examples can be found in, for example, U.S. Pat. No. 7,213,367, incorporated herein by reference.

Various additives, such as adherents, dispersants, surfactants, and nutrient and buffer ingredients, can also be included in the seed treatment formulation. Other conventional seed treatment additives include, but are not limited to: coating agents, wetting agents, buffering agents, and polysaccharides. At least one agriculturally acceptable carrier can be added to the seed treatment formulation such as water, solids, or dry powders. The dry powders can be derived from a variety of materials such as calcium carbonate, gypsum, vermiculite, talc, humus, activated charcoal, and various phosphorous compounds.

In some embodiments, the seed coating composition can include at least one filler, which is an organic or inorganic, natural or synthetic component with which the active components are combined to facilitate its application onto the seed. In aspects, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

In some embodiments, the seed treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the seed, or alternatively may be added as part of the seed coating composition of the disclosure.

In some embodiments, the formulation that is used to treat the seed in the present disclosure can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation can be about 0.5% to about 99% by weight (w/w), or 5-40%, or as otherwise formulated by those skilled in the art.

As mentioned above, other conventional inactive or inert ingredients can be incorporated into the formulation. Such inert ingredients include, but are not limited to: conventional sticking agents; dispersing agents such as methylcellulose, for example, serve as combined dispersant/sticking agents for use in seed treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the present disclosure can be found in McCutcheon's, vol. 1, “Emulsifiers and Detergents,” MC Publishing Company, Glen Rock, N.J., U.S.A., 1996, incorporated by reference herein.

The seed coating formulations of the present disclosure can be applied to seeds by a variety of methods, including, but not limited to: mixing in a container (e.g., a bottle or bag), mechanical application, tumbling, spraying, and immersion. A variety of active or inert material can be used for contacting seeds with microbial compositions according to the present disclosure.

In some embodiments, the amount of the composition of the disclosure used for the treatment of the seed will vary depending upon the type of seed and the type of active ingredients, but the treatment will include contacting the seeds with an agriculturally effective amount of the inventive composition.

As discussed above, an effective amount means that amount of the inventive composition that is sufficient to affect beneficial or desired results. An effective amount can be administered in one or more administrations.

In some embodiments, in addition to the coating layer, the seed may be treated with one or more of the following ingredients: other pesticides including fungicides and herbicides; herbicidal safeners; fertilizers and/or biocontrol agents. These ingredients may be added as a separate layer or alternatively may be added in the coating layer.

In some embodiments, the seed coating formulations of the present disclosure may be applied to the seeds using a variety of techniques and machines, such as fluidized bed techniques, the roller mill method, rotostatic seed treaters, and drum coaters. Other methods, such as spouted beds may also be useful.

The seeds may be pre-sized before coating. After coating, the seeds are typically dried and then transferred to a sizing machine for sizing. Such procedures are known in the art.

In some embodiments, the microorganism-treated seeds may also be enveloped with a film overcoating to protect the coating. Such overcoatings are known in the art and may be applied using fluidized bed and drum film coating techniques.

In other embodiments of the present disclosure, compositions according to the present disclosure can be introduced onto a seed by use of solid matrix priming. For example, a quantity of an inventive composition can be mixed with a solid matrix material and then the seed can be placed into contact with the solid matrix material for a period to allow the composition to be introduced to the seed. The seed can then optionally be separated from the solid matrix material and stored or used, or the mixture of solid matrix material plus seed can be stored or planted directly. Solid matrix materials which are useful in the present disclosure include polyacrylamide, starch, clay, silica, alumina, soil, sand, polyurea, polyacrylate, or any other material capable of absorbing or adsorbing the inventive composition for a time and releasing that composition into or onto the seed. It is useful to make sure that the inventive composition and the solid matrix material are compatible with each other. For example, the solid matrix material should be chosen so that it can release the composition at a reasonable rate, for example over a period of minutes, hours, or days.

The compositions described herein may be substantially confined within an object, for example an object selected from the group consisting of: bottle, jar, ampule, package, vessel, bag, box, bin, envelope, carton, container, silo, shipping container, truck bed, case, and the like.

Aspect of the instant disclosure include, without limitation:

Aspect 1: A synthetic composition comprising a genetically engineered microbe, wherein said genetically engineered microbe comprises at least one genetic mutation at at least one locus within its genome, wherein said genetic mutation alters the production of one or more secondary metabolite.

Aspect 2: The synthetic composition of Aspect 1, wherein the non-genetically engineered microbe from which the genetically engineered microbe was created is of the genus Bacillus.

Aspect 3: The synthetic composition of Aspect 1, wherein the non-genetically engineered microbe from which the genetically engineered microbe was created is of the strain Bacillus velezensis.

Aspect 4: The synthetic composition of Aspect 1, wherein the secondary metabolite is a cyclic lipopeptide or a polyketide.

Aspect 5: The synthetic composition of Aspect 1, wherein the cyclic lipopeptide is an Iturin.

Aspect 6: The synthetic composition of Aspect 1, wherein the cyclic lipopeptide is a Fengycin.

Aspect 7: The synthetic composition of Aspect 1, wherein the cyclic lipopeptide is a Surfactin.

Aspect 8: The synthetic composition of Aspect 1, wherein the polyketide is a Macrolactin.

Aspect 9: The synthetic composition of Aspect 1, wherein the polyketide is a Difficidin.

Aspect 10: The synthetic composition of Aspect 1, wherein the polyketide is a Baccilaene.

Aspect 11: The synthetic composition of Aspect 1, wherein the genetic mutation is a knockout, knock-in, disruption, insertion of at least one nucleotide, deletion of at least one nucleotide, replacement of at least one nucleotide, chemical modification of at least one nucleotide, molecular structure alteration of at least one nucleotide, downregulation of at least one gene, upregulation of at least one gene, and/or any combination and/or plurality of the preceding.

Aspect 12: The synthetic composition of Aspect 1, wherein the genetic mutation comprises the addition of a least one nucleotide.

Aspect 13: The synthetic composition of Aspect 1, wherein the genetic mutation comprises the deletion of at least one nucleotide.

Aspect 14: The synthetic composition of Aspect 1, wherein the genetic mutation comprises the chemical alteration of at least one nucleotide.

Aspect 15: The synthetic composition of Aspect 1, wherein the genetic mutation is a knockout, disruption, deletion, and/or downregulation of kinA.

Aspect 16: The synthetic composition of Aspect 11, wherein the disruption of kinA is accomplished by the insertion of an sfp gene operably linked to its native promoter.

Aspect 17: The synthetic composition of Aspect 1, wherein the genetic mutation is a knockout, disruption, deletion, and/or downregulation of thrC.

Aspect 18: The synthetic composition of Aspect 17, wherein the disruption of thrC is accomplished by the insertion of an sfp gene operably linked to its native promoter.

Aspect 19: The synthetic composition of Aspect 17, wherein the disruption of thrC is accomplished by the insertion of a degU gene.

Aspect 20: The synthetic composition of Aspect 17, wherein the disruption of thrC is accomplished by the insertion of a comA gene operably linked to its native promoter.

Aspect 21: The whole cell broth or exudate obtained from the synthetic composition of Aspect 1.

Aspect 22: A formulation comprising the synthetic composition of Aspect 1 or the whole cell broth or exudate of Aspect 21.

Aspect 23: The formulation of Aspect 22, wherein the synthetic composition is substantially purified.

Aspect 24: The formulation of Aspect 22, wherein the composition of (b) further comprises one or more formulation components.

Aspect 25: The formulation of Aspect 24, wherein the formulation component(s) is(are) selected from the group consisting of: a salt, a binder, a surface-active agent, a surfactant, a wetting agent, a dispersing agent, an emulsifying agent, a solubilizing agent, an organic solvent, a gelling agent, a thickening agent, an anti-settling agent, a preservative, a stabilizer, an anti-freezing compound, a plurality of any of the preceding, and any combination of the preceding.

Aspect 26: The method of Aspect 1, wherein the composition further comprises one or more additional agents selected from the group consisting of: a pesticide, an herbicide, a bactericide, a fungicide, an insecticide, a virucide, a miticide, a nematicide, an acaricide, a plant growth regulator, a rodenticide, an anti-algae agent, a biocontrol agent, a fertilizer, a biopesticide, a biostimulant, and any combination and/or plurality of the preceding.

Aspect 27: A method of controlling a pathogen, said method comprising applying to a target object the synthetic composition of Aspect 1 or the whole cell broth or exudate of Aspect 21.

Aspect 28: The method of Aspect 27, wherein the pathogen is a bacterium.

Aspect 29: The method of Aspect 27, wherein the pathogen is a fungus.

Aspect 30: The method of Aspect 27, wherein the target object is a cell.

Aspect 31: The method of Aspect 27, wherein the target object is a plant or plant part.

Aspect 32: The method of Aspect 27, wherein the target object is a seed.

Aspect 33: The method of Aspect 27, wherein the synthetic composition comprises an engineered microbe derived from the genus Bacillus.

Aspect 34: The method of Aspect 27, wherein the composition of (b) further comprises one or more formulation components.

Aspect 35: The method of Aspect 27, wherein the formulation component(s) is(are) selected from the group consisting of: a salt, a binder, a surface-active agent, a surfactant, a wetting agent, a dispersing agent, an emulsifying agent, a solubilizing agent, an organic solvent, a gelling agent, a thickening agent, an anti-settling agent, a preservative, a stabilizer, an anti-free compound, a plurality of any of the preceding, and any combination of the preceding.

Aspect 36: The method of Aspect 27, wherein the composition further comprises one or more additional agents selected from the group consisting of: a pesticide, an herbicide, a bactericide, a fungicide, an insecticide, a virucide, a miticide, a nematicide, an acaricide, a plant growth regulator, a rodenticide, an anti-algae agent, a biocontrol agent, a fertilizer, a biopesticide, a biostimulant, and any combination and/or plurality of the preceding.

Aspect 37: The method of Aspect 27, wherein the synthetic composition, whole cell broth, or exudate is applied to a plant or plant part prior to germination.

Aspect 38: The method of Aspect 27, wherein the synthetic composition, whole cell broth, or exudate is applied to a plant or plant part post-harvest.

Aspect 39: The method of Aspect 27, wherein the synthetic composition, whole cell broth, or exudate is applied to a plant or plant part during the vegetative phase of the plant.

Aspect 40: The method of Aspect 27, wherein the synthetic composition, whole cell broth, or exudate is applied to a plant or plant during the reproductive phase of the plant.

Aspect 41: The method of Aspect 27, wherein the synthetic composition, whole cell broth, or exudate is applied to a plant or plant part pre-harvest.

Aspect 42: The method of Aspect 31, wherein the plant is cultivated in a field, harvested, placed in storage, or distributed.

Aspect 43: The method of Aspect 31, wherein the plant part is a root, leaf, stem, flower, seed, bulb, or fruit.

Aspect 44: The method of Aspect 31, further comprising applying a plurality of compositions of (b) to the plant.

Aspect 45: A synthetic composition comprising:
(a) a plant or plant part, and
(b) the synthetic composition of Aspect 1 or the whole cell broth or exudate of Aspect 21.

Aspect 46: The synthetic composition of Aspect 45, wherein the synthetic composition is substantially confined within an object selected from the group consisting of: bottle, jar, ampule, package, vessel, bag, box, bin, envelope, carton, container, silo, shipping container, truck bed, and case.

Aspect 47: A plurality of synthetic compositions of Aspect 45, wherein each of the plurality are placed in a growth medium.

Aspect 48: The plurality of synthetic compositions of Aspect 47, wherein the growth medium is soil.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof.

All cited patents and publications referred to in this application are herein incorporated by reference in their entirety, for all purposes, to the same extent as if each were individually and specifically incorporated by reference. Mention of any reference, article, publication, patent, patent publication, and patent application cited herein is not, however, and should not be taken as, an acknowledgment or any form of suggestion that they constitute valid prior art or form part of the common general knowledge in any country in the world.

### EXAMPLES

The following are Examples of specific embodiments of some aspects of the invention. The Examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for. Abbreviations include: um=mm (micrometer); ul=ml (microliter); C=degrees Celsius; h=hours.

### Example 1: Genome Modifications of Different Bacterial Strains

Different genomic loci involved in secondary metabolite production, including those described below, were identified for gene editing. A summary of the edits and strains is given below in Table 1.

Note that strain nomenclature may optionally include a prefix before each strain number. For example, wild-type strain 102504 may optionally include the prefix “CM” (e.g., CM102504). A corresponding edited strain may optionally include a prefix “CE” or “CM” (e.g., CE102504-G5, CM14416-G2, etc.). The strain number, irrespective of the prefix, is a unique identifier. Genome-edited strains are indicated by the letter G following a dash (“-G”) and an edit number (e.g., CM14416-G2). A strain number (irrespective of prefix) and its “G number” is a unique identifier for a particular edited strain.

TABLE 1
Genomic Edits

| Strain | Edit | Description |
|---|---|---|
| 7084 | Wild Type | (no edit) |
| (7084-WT) | | |
| 7084-G67 | kinA::Psfp-sfp | Disruption of the kinA gene by inserting |
| | | the sfp gene and its native promoter |
| 7084-G68 | PcodY::PsigA | replacement of the codY promoter with |
| | | the sigA promoter |
| 7084-G75 | PcodY::PaprE | replacement of the codY promoter with |
| | | the aprE promoter |
| 7084-G76 | PcodY::Phag | replacement of the codY promoter with |
| | | the hag promoter |
| 7084-G77 | ΔkinA | Deletion of the kinA gene |
| 7084-G93 | ΔthrC | Deletion of the thrC gene |
| 7084-G95 | Δdif_ks1 | Deletion of the 1st ketide synthase |
| | | domain within the primary difficidin |
| | | synthase gene |
| 7084-G97 | thrC::PcomA- | Disruption of thrC by inserting the |
| | comA | comA gene and its native promoter |
| 7084-G103 | thrC::degU | Disruption of thrC by inserting a copy |
| | | of the degU gene |
| 7084-G105 | thrC::Psfp-sfp | Disruption of the thrC gene by inserting |
| | | the sfp gene and its native promoter |
| 102405 | Wild Type | (no edit) |
| 102504-G5 | ΔkinA | Deletion of the kinA gene |
| 14416 | Wild Type | (no edit) |
| 14416-G2 | ΔkinA | Deletion of the kinA gene |

The kinA gene encodes a member of a set of at least 3 histidine protein kinases that are involved in the phosphorylation of the master sporulation regulator, SpoOA. It phosphorylates the sporulation-regulatory proteins SpoOA and SpoOF. It also autophosphorylates in the presence of ATP. Under certain conditions, KinA has been shown to be the primary kinase which functions as the sensor kinase in a traditional two component signal transductions system to integrate input signals it receives to its cognate response regulator to promote sporulation.

The sfp gene encodes 4′ phosphopante theinyl transferase: an essential enzyme for secondary metabolite production. SFP activates the synthesis cascades of virtually all secondary metabolites, including surfactin, fengycins, iturins, plipastatin, difficidins, macrolactins, and bacillibactin.

The sigA gene encodes SigA, the vegetative sigma factor analogous to the sigma-70 sigma factor in E. coli that regulates the transcription of primary metabolism during vegetative cell growth.

The codY gene encodes CodY, a global transcriptional regulator in low-G+C Gram-positive bacteria that is responsive to GTP and branched-chain amino acids. By interacting with its two cofactors, it is able to sense the nutritional and energetic status of the cell and respond by regulating expression of adaptive genetic programs including secondary metabolite production.

The thrC gene encodes a threonine synthase enzyme involved in threonine biosynthesis. Strains carrying disruptions in thrC are unable to grow without supplementation of threonine in culture media.

The gene aprE encodes AprE, an extracellular alkaline serine protease (otherwise known as subtilisin E). The aprE promoter, PaprE, was chosen due to prior evidence indicating that it is a strong constitutive promoter.

The hag gene encodes the flagellin subunit protein, Hag, which polymerizes into the filament portion of the flagellum. Transcription of hag is dependent on the formation of the flagellar basal body, but this requirement may be bypassed by overexpression of sigD; an alternative sigma factor that directs RNA polymerase activity to promoters that are recognized and are bound by SigD. Hag is similar to FliC of E. coli.

The degU gene encodes DegU, a response regulator within a two-component signal transduction system with DegS involved in degradative enzyme and competence regulation. DegU activity has been shown to positively regulate production of secondary metabolite production including cyclic lipopeptides.

It is thought that disruption of the fadA_ks1 locus leads to loss of difficidin production.

The comA gene encodes ComA, a transcription factor that promotes transcription of late competence genes and surfactin production. ComA has been shown to regulate the quorum response in numerous Gram-positive bacteria.

### Transformation and Sequence Verification:

1-5 ul of the Gibson assembly mixture was added to 50 ul of freshly thawed, chemically competent E. coli DH5 α cells and finger vortexed. The cell-plasmid mixture was incubated on ice for 30 minutes, heat shocked at 42 C for 30 seconds, then moved back to ice for a further 5 minutes. 1 ml SOC (Super-Optimal Catabolism) medium was added, and the cells were incubated at 37 C with 200 RPM shaking for 60-90 minutes for recovery. Dilutions of the recovery culture were plated onto LB agar plates supplemented with 100 ug/ul ampicillin and incubated overnight at 37 C.

The plasmid region comprising the assembled inserts was amplified from several recovered colonies via colony PCR using Q5 high-fidelity polymerase and the PCR products were run on an agarose gel to confirm the expected sized product. Appropriately-sized PCR products were sent for Sanger sequencing to confirm proper assembly and lack of any off-target mutations in the editing cassette.

### Preparation for Conjugation

Colonies confirmed to harbor the correct plasmid were inoculated into LB broth supplemented with 100 ug/ul ampicillin and grown overnight at 37 C and 200 RPM in a shaker/incubator. The plasmid was purified from the overnight culture and transformed into conjugation donor strain E. coli BW29427 via electroporation.

1 ul of the purified plasmid was combined with 50 ul of freshly thawed E. coli BW29427 electrocompetent cells and incubated for 5 minutes on ice. The cell-plasmid mixture was transferred into a pre-chilled 1 mm electroporation cuvette on ice. Using an electroporator, a 1800 V, 25 uF, 200 Ohm charge was applied to the cuvette, and the sample was immediately resuspended in 1 ml SOC medium supplemented with 0.3 mM 2,6-diaminopimelic acid (DAP). The resuspended cells were incubated at 37 C with 200 RPM shaking for 60-90 minutes for recovery. Dilutions of the recovery culture were plated onto LB agar plates supplemented with 100 ug/ul ampicillin and 0.3 mM 2,6-diaminopimelic acid and incubated overnight at 37 C. Recovered transformants were used as donor strains for conjugation.

### Conjugation

Recipient strains were inoculated into 5 ml Tryptic Soy Broth (TSB) medium in 50 ml conical tubes and grown overnight at 30 C and 200 RPM shaking. Donor E. coli BW29427 harboring the plasmid to be mobilized was inoculated into 5 ml LB medium supplemented with 100 ug/ul ampicillin and 0.3 mM 2,6-diaminopimelic acid (DAP) and grown overnight at 37 C and 200 RPM shaking.

1 ml aliquots of the overnight donor and recipient cultures were spun down, washed in sterile water, combined and spotted onto the surface of LB agar plates supplemented with 0.3 mM DAP for conjugative mating. Mating plates were incubated overnight at 25 C, the permissive temperature for replication of pMMDmob in Gram positive recipient strains.

Mating mixtures were resuspended by the addition of 1 ml phosphate buffer salts (PBS) over the top of the spot and agitated with a sterile L-spreader. Resuspensions were collected in microcentrifuge tubes, washed, and resuspended in 100 ul of PBS. The concentrated cells were spread over TSA plates supplemented with MLS (25 ug/ml lincomycin, 1 μg/ml erythromycin) with no DAP added and incubated for 48-72 hours at 25 C until the appearance of transconjugant colonies.

### Plasmid Integration

Recovered transconjugants were inoculated into 5 ml TSB medium supplemented with mls and grown for 48 hours at 25 C with 200 RPM shaking, or until turbid. Dilutions of the culture were plated onto TSA+mls plates and incubated overnight at 37 C, the restrictive temperature for plasmid replication, until the appearance of integrated colonies.

### Plasmid Excision

Integrated colonies were inoculated into 5 ml TSB medium supplemented with mls and incubated overnight at 37 C with 200 RPM shaking. 5 ul of the overnight culture was diluted into 5 ml fresh TSB medium without antibiotics and grown overnight at 25 C, 200 RPM shaking. Subculturing of 5 ul overnight culture into 5 ml fresh TSB at 25° C., 200 RPM shaking was repeated twice, for a total of three rounds of subculturing. Dilutions of the round three overnight culture were plated onto R2A plates lacking antibiotics and incubated at 30° C. overnight.

Excision of the plasmid was confirmed by picking individual colonies from the R2A plate and re-plating them in a grid format onto agar plates with and without MLS. Both plates were incubated at 30 C for 24-48 hours until the appearance of colonies. Colonies that grew when plated onto the plate without MLS, but not on the plate containing MLS, were confirmed to have excised and lost the plasmid.

### Confirmation of Editing

The edit region was amplified from putative edited strains via colony PCR, and the presence of the proper edit was confirmed by the size of the band when run on an agarose gel (when possible), and/or by Sanger sequencing. The absence of the plasmid backbone was confirmed by PCR assaying of the mls resistance cassette. Colonies yielding a band for the mls cassette were deemed to not be proper edits. Other methods for sequencing and verification of edits may be used, such as whole genome sequencing.

Sequence results were analyzed to differentiate colonies that had excised to wild type from properly edited colonies, and the sequences were checked for off-target mutations.

Colonies that were sequenced as proper edits without off-target mutations were made available for bioassay analysis.

### Example 2: Modification of Bacillus velezensis Strain 7084

Bacillus velezensis Strain 7084 (NRRL Deposit No. NRRL B-67810 deposited 3 Jul. 2019) was used as a parental strain that was edited to obtain genome-edited strains with improved composition production for antifungal and/or antibacterial activity(ies).

kinA::Psfp-sfp

Wild-Type parental Strain 7084 was modified as follows: the sfp gene encoding 4′ phosphopante theinyl transferase and its corresponding upstream promoter (Psfp) was copied using Q5 high-fidelity polymerase mediated PCR from the 7084 chromosome and inserted into the kinA gene, disrupting the kinA open reading frame. The resultant strain (7084-G67) contained a null mutation of kinA and a duplication of the native sfp gene and sfp promoter at the kinA locus.

To generate plasmid pAP41 used to insert Psfp-sfp at the kinA locus, PCR products containing the Psfp promoter and sfp gene, the region upstream of kinA, and the region downstream of kinA were amplified using Q5 high-fidelity polymerase mediated PCR from 7084 using appropriate primer pairs. Primer pairs contained appropriate Gibson assembly overhangs to facilitate isothermal assembly of the fragments to each other and to the linearized cloning vector, pMMDmob, such that the final version comprised the region upstream of kinA on the left flank of the Psfp-sfp fragment and the region downstream of kinA on the right flank of the Psfp-sfp fragment. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3:3 backbone:insert:insert:insert molar ratio in a 10 ul volume, then adding 10 ul 2× Gibson Reagent. The reaction was incubated at 50 C for 60 minutes then used for transformation into E. coli DH5 α.

### PcodY::PsigA

Wild-Type parental Strain 7084 was modified as follows: the codY promoter (PcodY) was replaced with the sigA promoter (PsigA). Promoters for each gene were designated as 200 bp sequence immediately upstream of each gene's start codon.

To generate plasmid pAP49 used conduct the exchange of PcodY with PsigA, PCR products containing PsigA, the region upstream of PcodY, and the region downstream of PcodY were amplified using Q5 high-fidelity polymerase mediated PCR from 7084 using appropriate primer pairs. Primer pairs contained appropriate Gibson assembly overhangs to facilitate isothermal assembly of the fragments to each other and to the linearized cloning vector, pMMDmob, such that the final version (resultant Strain 7084-G68) comprised the region upstream of PcodY on the left flank of the PsigA fragment and the region downstream of PcodY on the right flank of the PsigA fragment. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3 backbone:insert:insert:insert molar ratio in a 10 ul volume, then adding 10 ul 2× Gibson Reagent. The reaction was incubated at 50 C for 60 minutes then used for transformation into E. coli DH5a.

ΔkinA

Wild-Type parental Strain 7084 was modified as follows: the kinA gene was targeted for in frame marker-less deletion. The resultant strain (7084-G77) comprised a kinA knockout.

To generate plasmid pAP18 used to create the in-frame marker-less deletion in 7084, PCR products containing the region upstream of kinA and the region downstream of kinA were amplified using Q5 high-fidelity polymerase from 7084 using appropriate primer pairs. Primer pairs contained appropriate Gibson assembly overhangs to facilitate isothermal assembly of the fragments to each other and to the linearized cloning vector, pMMDmob, such that the final construct comprised the region upstream of kinA directly ligated to the region downstream of kinA such that the reading frame was maintained from the kinA start codon to the kinA stop codon while also eliminating 1543 bp of the middle of the kinA coding sequence. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3 backbone:insert:insert molar ratio in a 10 ul volume, then adding 10 ul 2× Gibson Reagent. The reaction was incubated at 50 C for 60 minutes then used for transformation into E. coli DH5a.

thrC::Psfp-sfp

Wild-Type parental Strain 7084 was modified as follows: the sfp gene encoding 4′ phosphopante theinyl transferase and its corresponding upstream promoter (Psfp) was copied using Q5 high-fidelity polymerase mediated PCR from the 7084 chromosome and inserted into the thrC gene, disrupting the thrC open reading frame. The resultant strain (7084-G105) contained a null mutation of thrC and a duplication of the native sfp gene and sfp promoter at the thrC locus.

### PcodY::PaprE

Wild-Type parental strain 7084 was modified as follows: the codY promoter (PcodY) was replaced with the aprE promoter (PaprE). Promoters for each gene were designated as 200 bp sequence immediately upstream of each gene's start codon. The resultant strain was 7084-G75.

### PcodY::Phag

Wild-Type parental Strain 7084 was modified as follows: the codY promoter (PcodY) was replaced with the hag promoter (Phag). Promoters for each gene were designated as 200 bp sequence immediately upstream of each gene's start codon. The resultant strain was 7084-G76.

thrC::degU

Wild-Type parental Strain 7084 was modified as follows: the degU gene without its corresponding promoter was copied using Q5 high-fidelity polymerase mediated PCR from the 7084 chromosome and inserted into the thrC gene, disrupting the thrC open reading frame. The resultant strain (7084-G103) comprised a null mutation of thrC and a duplication of the native degU under the transcriptional control of the thrC promoter.

To generate plasmid pAT5 used insert degU at the thrC locus, PCR products containing the degU fragment, the region upstream of thrC, and the region downstream of thrC were amplified using Q5 high-fidelity polymerase mediated PCR from 7084 using appropriate primer pairs. The degU promoter was excluded from the degU fragment because the complete degU transcription cassette was toxic to E. coli. Primer pairs contained appropriate Gibson assembly overhangs to facilitate isothermal assembly of the fragments to each other and to the linearized cloning vector, pMMDmob, such that the final version would consist of the region upstream of thrC on the left flank of the degU fragment and the region downstream of thrC on the right flank of the degU fragment. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3:3 backbone:insert:insert:insert molar ratio in a 10 ul volume, then adding 10 ul 2× Gibson Reagent. The reaction was incubated at 50 C for 60 minutes then used for transformation into E. coli DH5a.

Δdif_ks1

Wild-Type parental Strain 7084 was modified as follows: the difficidin gene cluster gene was targeted for in frame marker-less deletion at the first ketide synthase domain within the first difficidin synthesis gene in the cluster, fadA, resulting in Strain 7084-G95.

To generate plasmid pAP62 used to create the in-frame marker-less deletion in 7084, PCR products containing the region upstream of the first ketide synthase domain in fadA and the region downstream of the first ketide synthase domain in fadA were amplified using Q5 high-fidelity polymerase from Strain 7084 using appropriate primer pairs. Primer pairs contained appropriate Gibson assembly overhangs to facilitate isothermal assembly of the fragments to each other and to the linearized cloning vector, pMMDmob, such that the final construct would consist of the region upstream of the first ketide synthase domain in fadA directly ligated to the region downstream of the first ketide synthase domain in fadA such that the reading frame was maintained from the fadA start codon to the fadA stop codon while also eliminating 377 bp of the fadA coding sequence. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3 backbone:insert:insert molar ratio in a 10 ul volume, then adding 10 ul 2× Gibson Reagent. The reaction was incubated at 50 C for 60 minutes then used for transformation into E. coli DH5a.

thrC::PcomA-comA

Wild-Type parental Strain 7084 was modified as follows: the comA gene and its corresponding upstream promoter (PcomA) was copied using Q5 high-fidelity polymerase mediated PCR from the 7084 chromosome and inserted into the thrC gene, disrupting the thrC open reading frame. The resultant strain (7084-G97) contained a null mutation of thrC and a duplication of the native comA gene and its promoter at the thrC locus.

To generate plasmid pAP58 used to insert PcomA-comA at the thrC locus, PCR products containing PcomA-comA, the region upstream of thrC, and the region downstream of thrC were amplified using Q5 high-fidelity polymerase mediated PCR from 7084 using appropriate primer pairs. Primer pairs contained appropriate Gibson assembly overhangs to facilitate isothermal assembly of the fragments to each other and to the linearized cloning vector, pMMDmob, such that the final version would consist of the region upstream of thrC on the left flank of the PcomA-comA fragment and the region downstream of thrC on the right flank of the PcomA-comA fragment. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3:3 backbone:insert:insert:insert molar ratio in a 10 ul volume, then adding 10 ul 2× Gibson Reagent. The reaction was incubated at 50 C for 60 minutes then used for transformation into E. coli DH5a.

ΔthrC

Wild-Type parental Strain 7084 was modified as follows: the thrC gene was targeted for in-frame marker-less deletion, resulting in Strain 7084-G93.

To generate plasmid pAP60 used to create the in-frame marker-less deletion in 7084, PCR products containing the region upstream of thrC and the region downstream of thrC were amplified using Q5 high-fidelity polymerase from 7084 using appropriate primer pairs. Primer pairs contained appropriate Gibson assembly overhangs to facilitate isothermal assembly of the fragments to each other and to the linearized cloning vector, pMMDmob, such that the final construct would consist of the region upstream of thrC directly ligated to the region downstream of thrC such that the reading frame was maintained from the thrC start codon to the thrC stop codon while also eliminating 674 bp of the middle of the thrC coding sequence. pMMDmob was linearized using the appropriate primer pair and gel purified. DNA fragments consisting of the above regions were gel purified.

Gibson assembly was performed by combining approximately 100 ng of digested pMMDmob with the insert fragments in a 1:3:3 backbone:insert:insert molar ratio in a 10 ul volume, then adding 10 ul 2× Gibson Reagent. The reaction was incubated at 50 C for 60 minutes then used for transformation into E. coli DH5a.

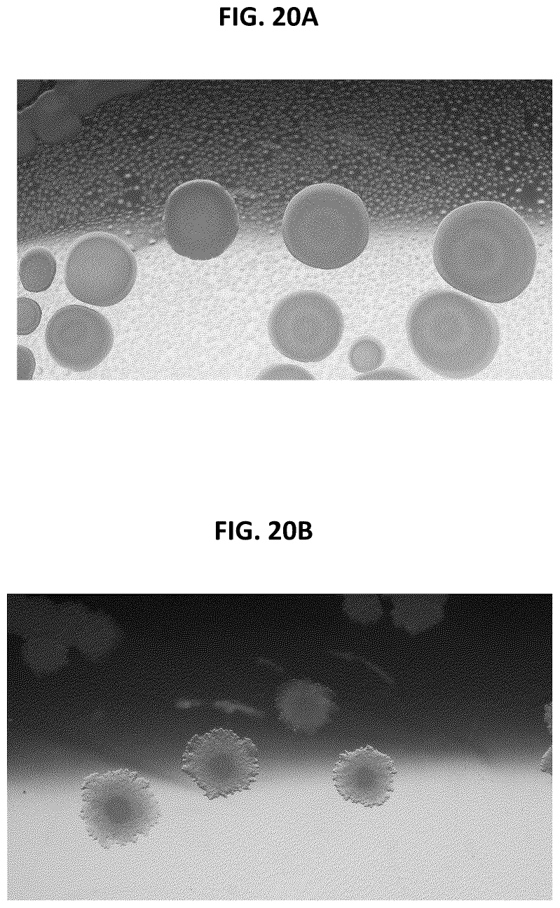

The wild-type Strain 7084 exhibited two different morphologies in flask cell culture: a majority of colonies exhibited a primary morphology of smooth, rounded cultures (FIG. 20A) while some colonies exhibited an alternate morphology of serrated-edged cultures (FIG. 20B). Most of the edited strains also exhibited a majority of the primary colony morphology, with some colonies of the alternate morphology. 7084-G67 (kinA::Psfp-sfp) exhibited only the alternative, serrated-edged colony morphology. 7084-G77 (kinA KO) exhibited the smooth-edged morphology.

### Example 3: Modification of Bacillus velezensis Strain 14416

Bacillus velezensis Strain 14416 was used as the parental strain that was edited to obtain genome-edited strains with improved composition production for antifungal and/or antibacterial activity(ies).

The kinA gene of CM14416 was targeted for in-frame marker-less deletion using an editing plasmid designed for a close relative of CM14416, CM7084. The resultant strain (CE14416-G2) comprised a kinA gene deletion through the previously referenced method known as “blind editing”.

To generate plasmid pAP18 used to create the in-frame marker-less deletion in CE14416-G2, PCR products approximately 1000 bp containing the region upstream of kinA and the region downstream of kinA were amplified using Q5 high-fidelity polymerase from CM7084 using appropriate primer pairs. Primer pairs contained appropriate Gibson assembly overhangs to facilitate isothermal assembly of the fragments to each other and to the linearized cloning vector, pMMDmob, such that the final construct comprised the region upstream of kinA directly ligated to the region downstream of kinA such that the reading frame was maintained from the kinA start codon to the kinA stop codon while also eliminating 1543 bp of the middle of the kinA coding sequence. pMMDmob was linearized using the EcoR1 and BamH1 endonuclease enzymes and gel purified. DNA fragments consisting of the above regions were gel purified.

Gibson assembly was performed by combining approximately 100 ng of digested pMMDmob with the insert fragments in a 1:3:3 backbone:insert:insert molar ratio in a 10 ul volume, then adding 10 ul 2× Gibson Reagent. The reaction was incubated at 50 C for 60 minutes then used for transformation into E. coli DH5a.

The assembled plasmid was transformed into the conjugation donor strain E. coli BW29427, and then conjugated into CM14416. CM14416 colonies resistant to the antibiotic resistance marker on pAP18 (MLS) were selected for integration into and subsequent excision of the plasmid from the host chromosome. The resulting strain was confirmed to contain a markerless in-frame deletion of kinA. Subsequent Sanger and illumina sequencing confirmed no other mutations were generated through this process. The resultant strain was CE14416-G2.

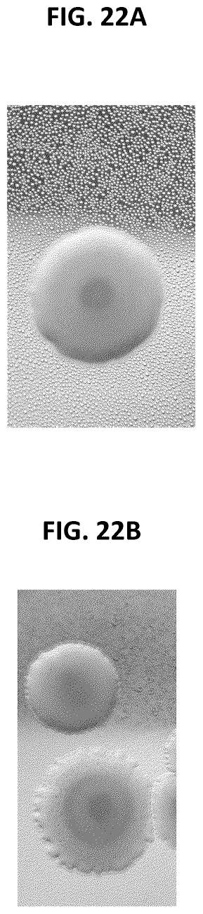

The Bacillus velezensis wild-type Strain 14416 exhibited a mostly smooth-edged morphology (FIG. 22A), and the edited strain 14416-G2 exhibited a serrated-edged morphology (FIG. 22B).

### Example 4: Modification of Paenibacillus polymyxa Strain 102504

Paenibacillus polymyxa Strain 102504 was used as the parental strain that was edited to obtain genome-edited strains with improved composition production for antifungal and/or antibacterial activity(ies).

The kinA gene was targeted for in-frame marker-less deletion. The resultant strain (CE102504-G5) comprised a kinA gene deletion within the CM102504 chromosome. To generate plasmid pAP77 used to create the in-frame marker-less deletion in CE102504-G5, PCR products approximately 1000 bp containing the region upstream of kinA and the region downstream of kinA were amplified using Q5 high-fidelity polymerase from CM102504 using appropriate primer pairs. Primer pairs contained appropriate Gibson assembly overhangs to facilitate isothermal assembly of the fragments to each other and to the linearized cloning vector, pMMDmob, such that the final construct comprised the region upstream of kinA directly ligated to the region downstream of kinA such that the reading frame was maintained from the kinA start codon to the kinA stop codon while also eliminating 1280 bp of the middle of the kinA coding sequence. pMMDmob was linearized using the EcoR1 and BamH1 endonuclease enzymes and gel purified. DNA fragments consisting of the above regions were gel purified. Gibson assembly was performed by combining approximately 100 ng of digested pMMDmob with the insert fragments in a 1:3:3 backbone:insert:insert molar ratio in a 10 ul volume, then adding 10 ul 2× Gibson reagent. The reaction was incubated at 50 C for 60 minutes then used for transformation into E. coli DH5a. The resulting strain was transformed into the conjugation donor strain E. coli BW29427, and then conjugated into CM102504. CM102504 colonies resistant to the antibiotic resistance marker on pAP77 (MLS) were selected for integration into and subsequent excision of the plasmid from the host chromosome. The resulting strain was confirmed to contain a markerless in-frame deletion of kinA. Subsequent Sanger and illumina sequencing confirmed no other mutations were generated through this process. The resultant strain was CE102504-G5.

### Example 5: Culture and Analysis of Genome Edited Bacteria

Primary cultures of the Bacillus velezensis wild type and edited strains were grown in 5 mL LB overnight at 30 C at 200 RPM. Secondary cultures were grown in a flask in 50 mL FM4 for 5 days at 30 C at 200 RPM. Samples were obtained of each culture for purity and HPLC assays.

The metabolite profiles of the supernatant samples of the cultures of interest were generated by subjecting the samples to separation by high-performance liquid chromatography using the method summarized in Table 2A (cyclic lipopeptides) and Table 3B (polyketides). Reference retention times used for identifying peaks are listed in Tables 2A (cyclic lipopeptides) and 3B (polyketides).

TABLE 2A
HPLC parameters for Cyclic Lipopeptides (CLPs)

| | | |
|---|---|---|
| Column | Stationary Phase | C18 |
| | Particle Size | 5 uM |
| | Pore Size | 120 A |
| | Column Diameter | 4.6 mm |
| | Column Length | 150 mm |
| | Column Temperature | 25° C. |

| | |
|---|---|
| Mobile Phase Solvent A | Water with 0.01% trifluoroacetic acid |
| Mobile Phase Solvent B | Acetonitrile with 0.01% trifluoroacetic acid |
| Gradient | 0-3 min: 40% solvent B; |
| | 3-8 min: 40-50% solvent B linear gradient; |
| | 8-13 min: 50-80% solvent B linear gradient; |
| | 13-30 min: 80-100% solvent B linear gradient |
| Gradient Runtime | 30 minutes |
| Flow Rate | 0.8 mL/min |
| Wavelength | 205 nm |

TABLE 2B
HPLC parameters for Polyketides (PKs)

| | | |
|---|---|---|
| Column | Stationary Phase | C18 |
| | Particle Size | 5 uM |
| | Pore Size | 120 A |
| | Column Diameter | 4.6 mm |
| | Column Length | 150 mm |
| | Column Temperature | 30° C. |

| | |
|---|---|
| Mobile Phase Solvent A | Water with 0.01% trifluoroacetic acid |
| Mobile Phase Solvent B | Acetonitrile with 0.01% trifluoroacetic acid |
| Gradient | 0-12 min: 20-95% solvent B linear gradient; |
| | 12-17 min: 95% solvent B |
| Gradient Runtime | 17 minutes |
| Flow Rate | 1.0 mL/min |
| Wavelength | 225 nm |

TABLE 3A
Retention Times of Cyclic Lipopeptides

| Retention Time (minutes) | Composition ID |
|---|---|
| 4.6 to 10.3 | Iturins |
| 10.3-17.3 | Fengycins |
| 17.3 to end | Surfactins |

TABLE 3B
Retention Times of Polyketides

| Retention | |
|---|---|
| Time (minutes) | Composition ID |
| 4.8 | Baccilaene |
| 5.6 | Macrolactin 1 |
| 5.7 | Macrolactin 2 |
| 6.2 | Macrolactin 3 |
| 6.6 | Macrolactin 4 |
| 8.2 | Macrolactin 5 |
| 12.8 | Difficidin 1 |
| 13.1 | Difficidin 2 |
| 13.6 | Difficidin 3 |

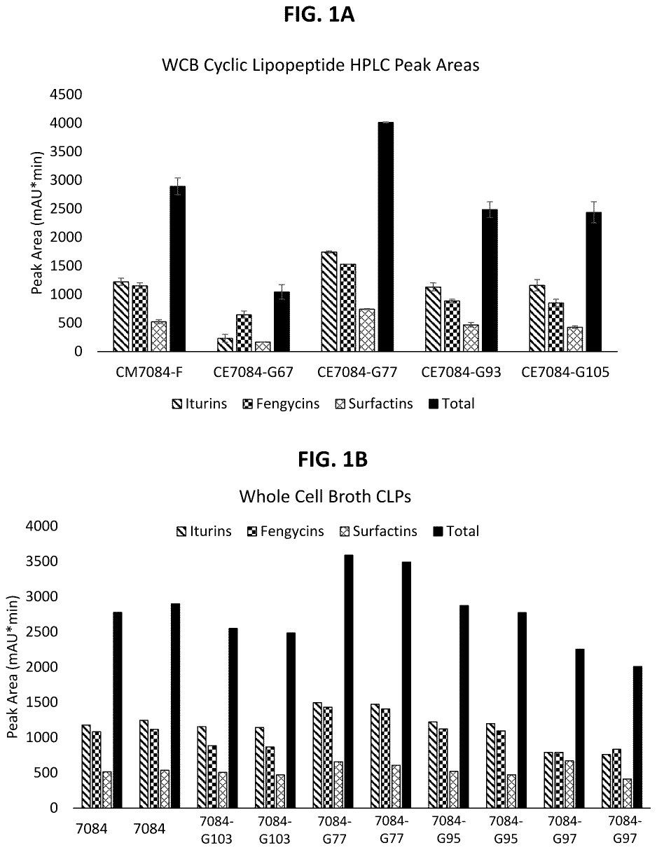

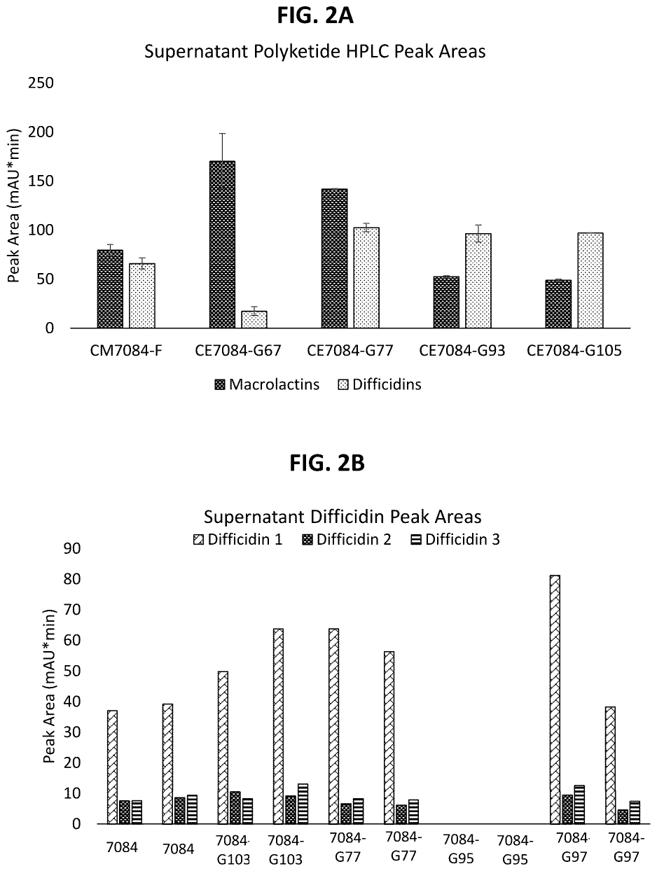

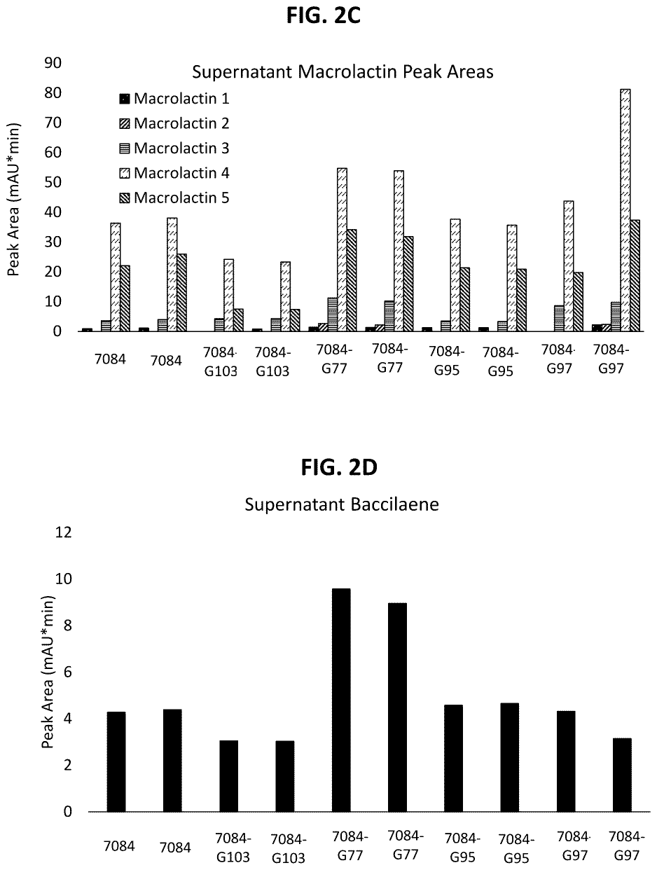

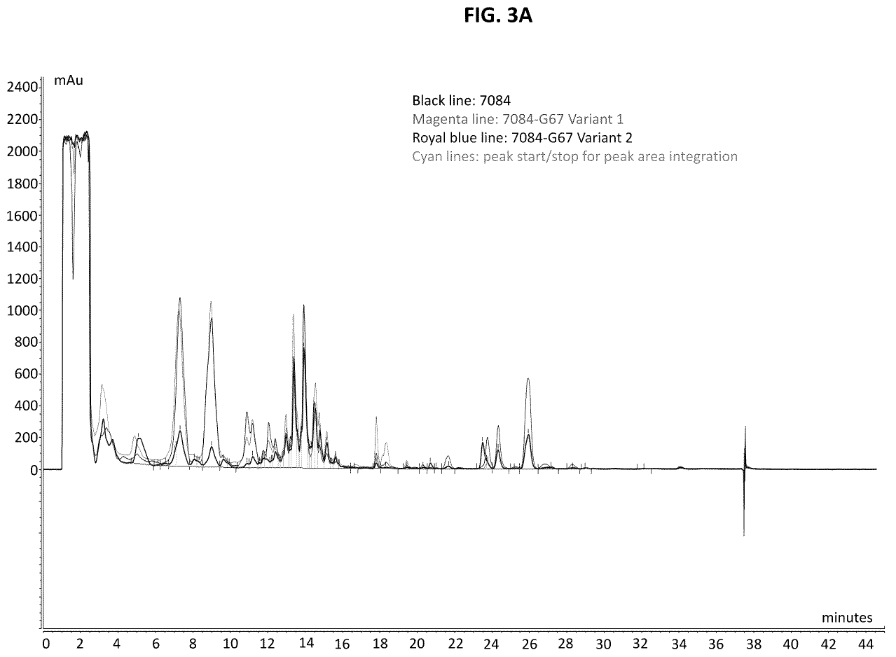

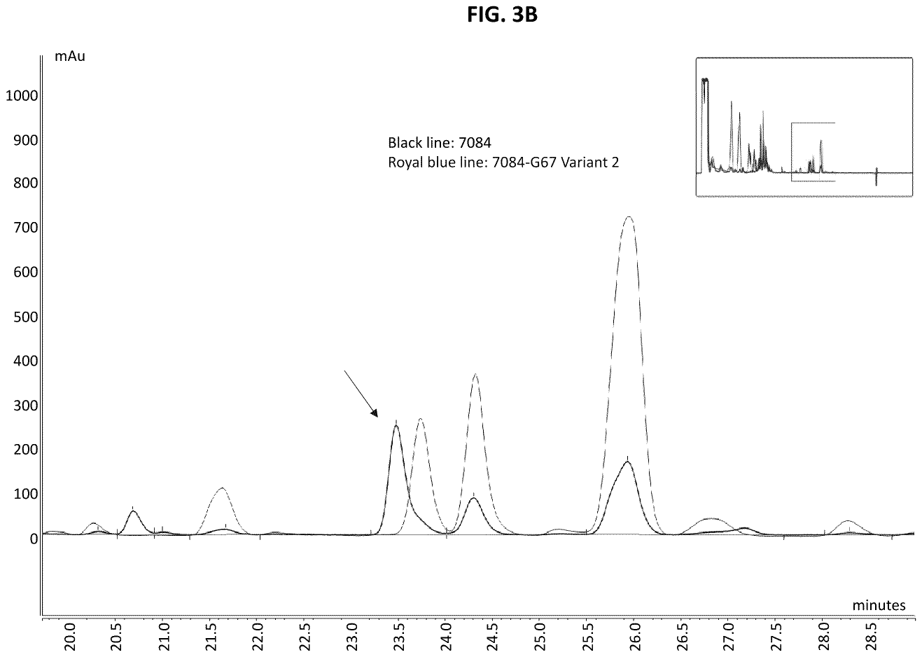

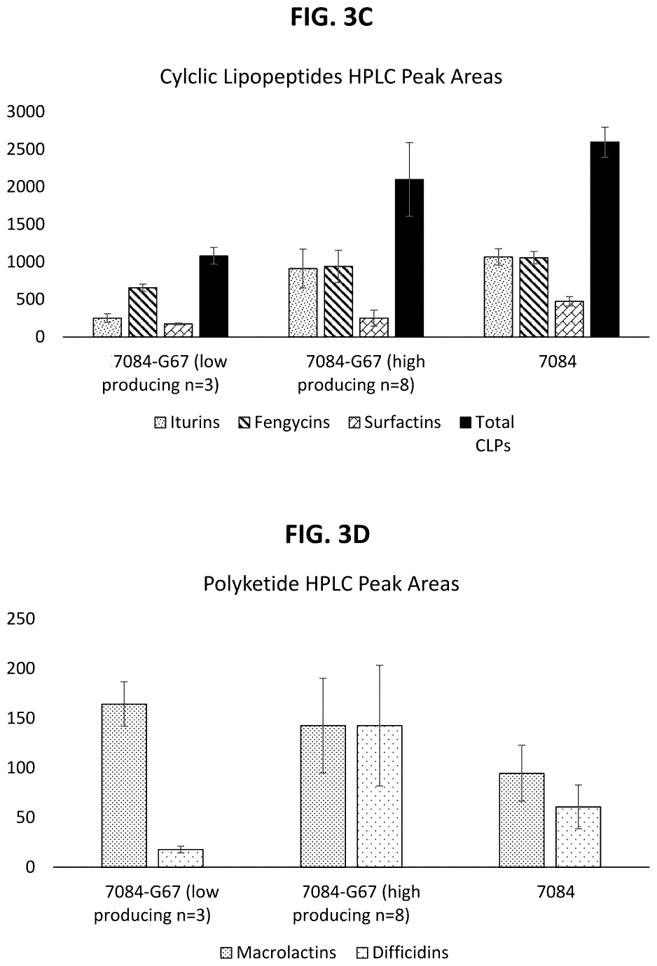

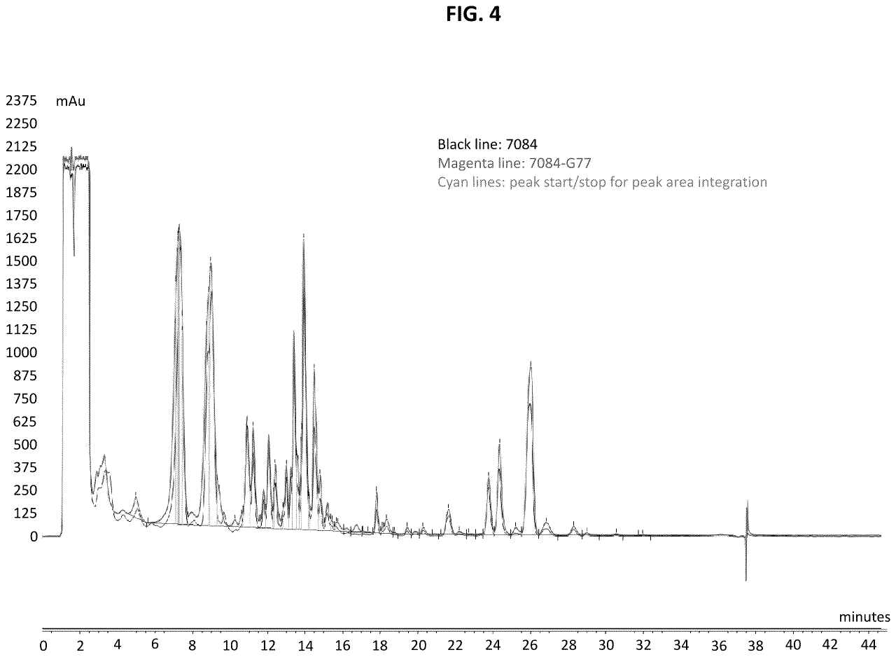

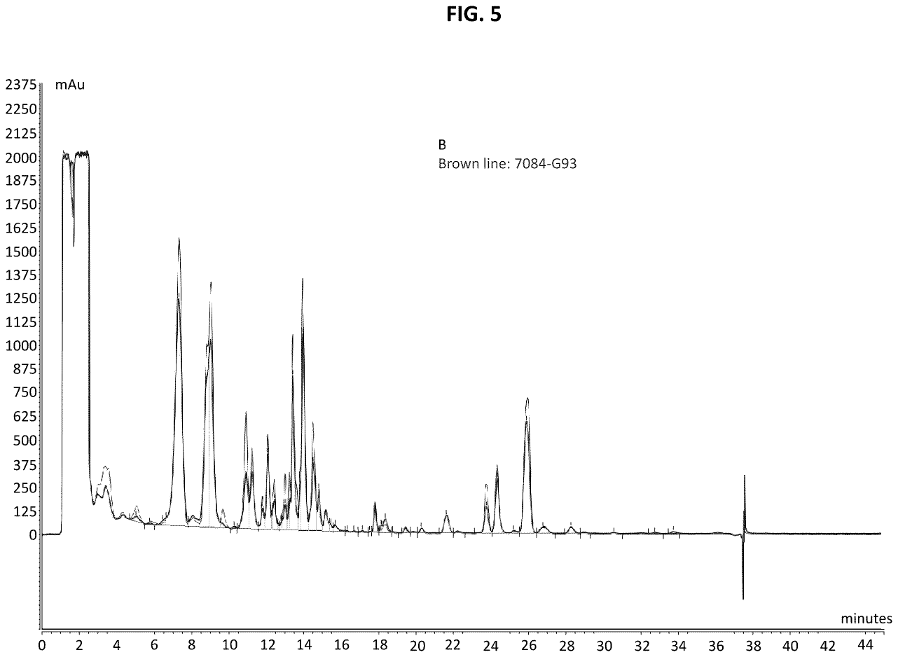

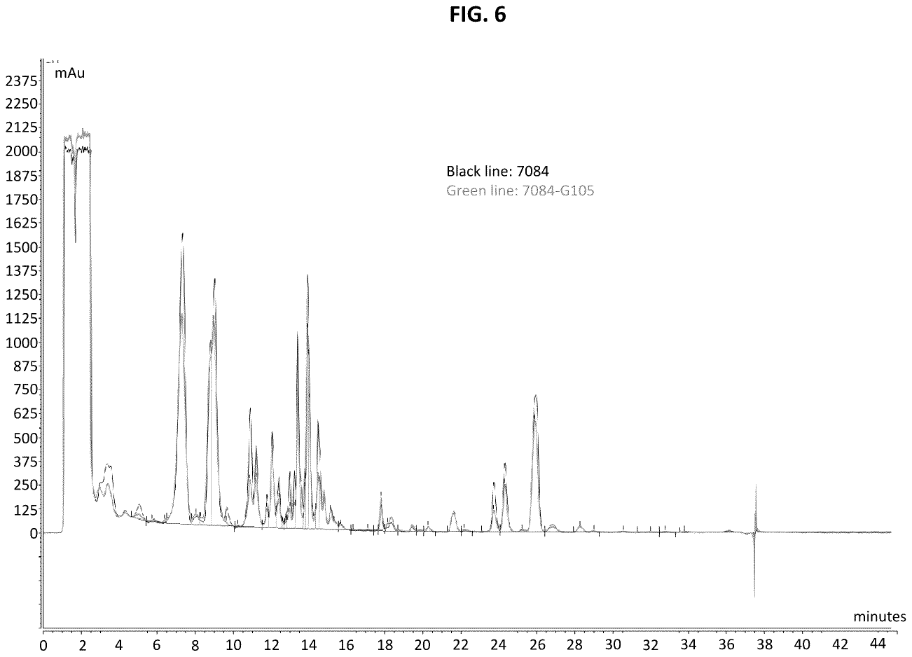

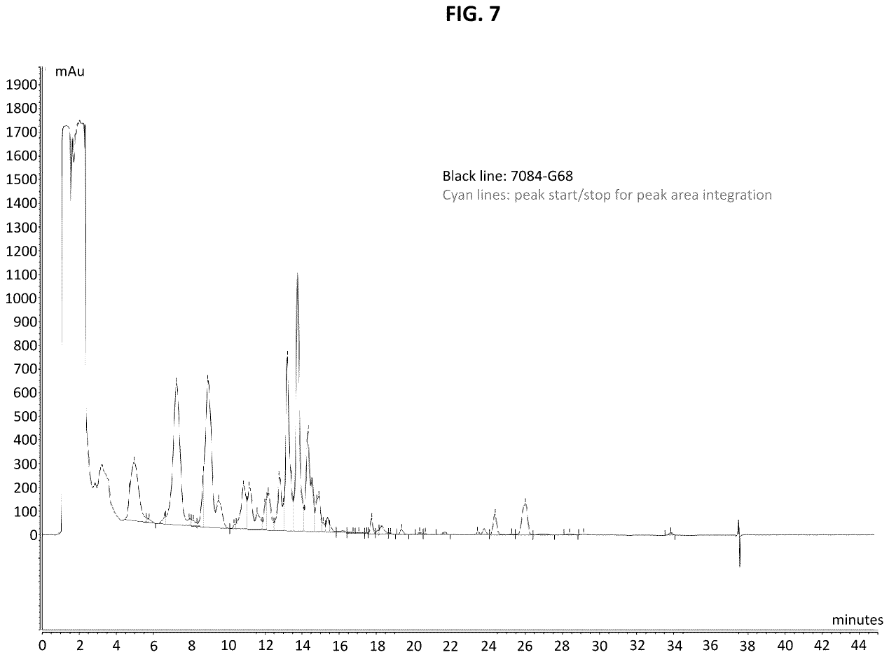

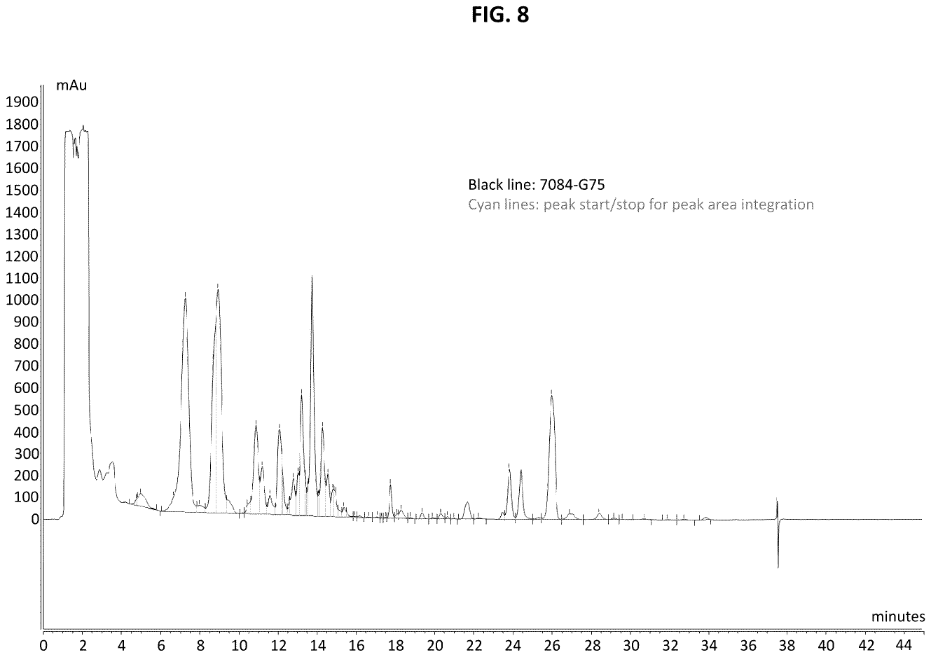

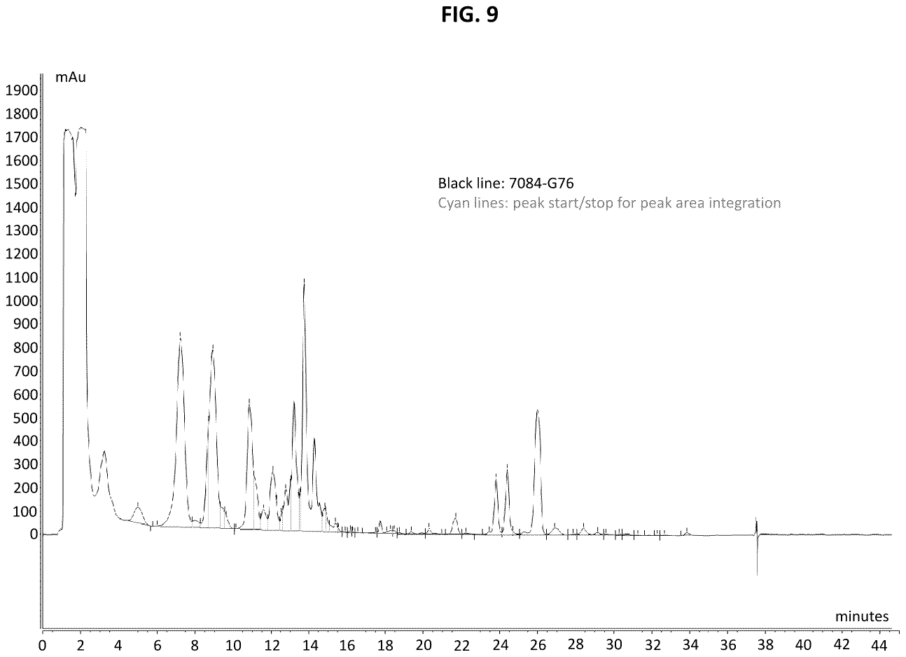

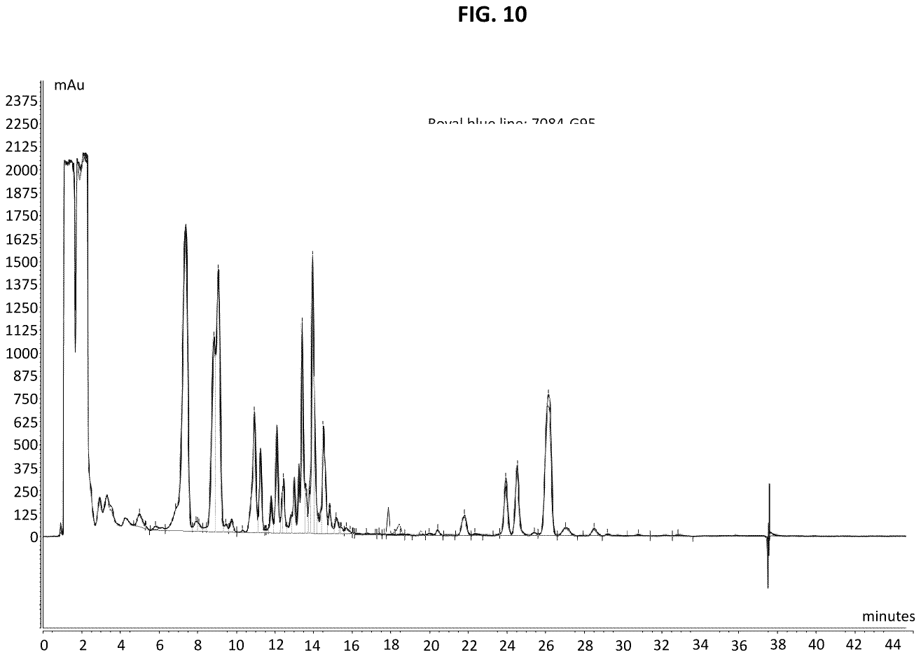

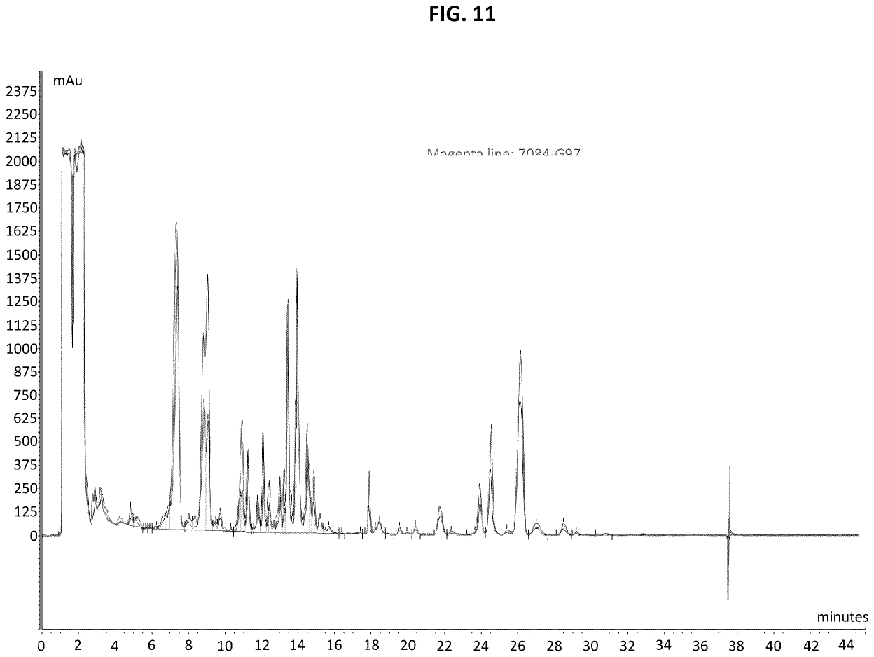

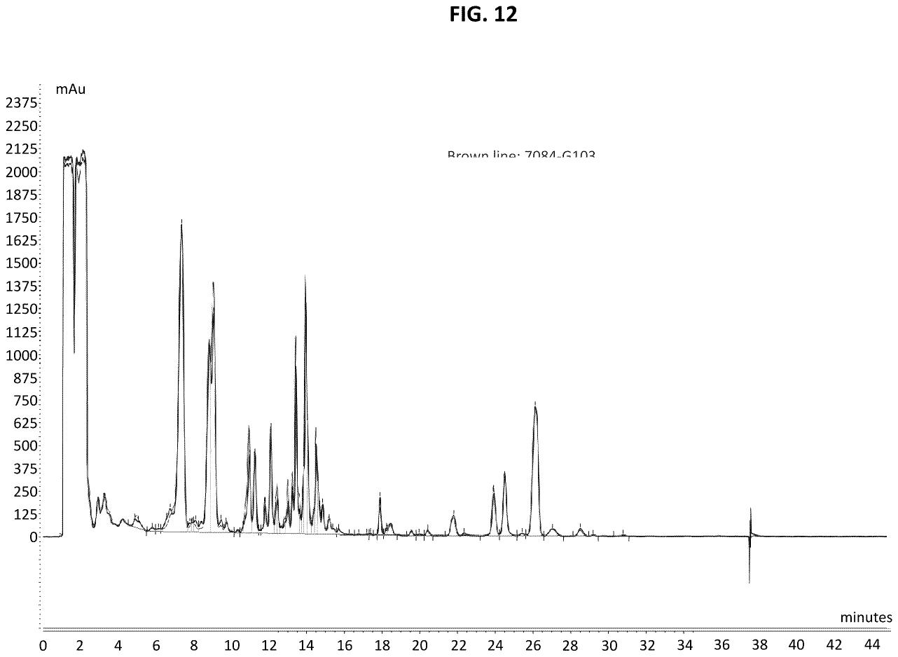

Cyclic lipopeptide profiles of wild type and genome-edited strains of 7084 are given in FIGS. 1A and 1B. Polyketide profiles of the wild type and genome-edited strains are given in FIGS. 2A-2D. Chromatograms are given in FIGS. 3-12. Genome edited strain 7084-G67 produced two different profiles across 11 different HPLC runs. Eight of the 11 runs produced a “high producing” variant, or Variant 1 (shown as the magenta line of FIG. 3A). Three of the 11 runs produced a “low producing” variant, or Variant 2 (shown as the royal blue line of FIG. 3A). Variant 2 also exhibited a shift in Surfactin peak retention times, shown in FIG. 3B. Comparisons of composition production for the two different variant productions of G67 and the WT strain are shown in FIG. 3C (cyclic lipopeptides) and FIG. 3D (polyketides).

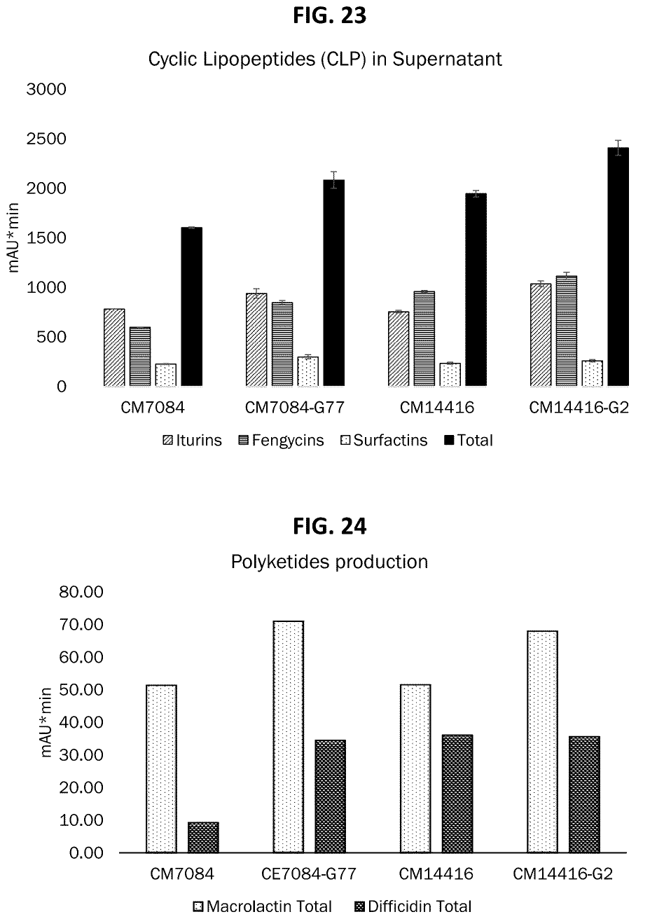

CLPs in supernatants of Strains 7084, 7084-G2, 14416, and 14416-G2 are shown in FIG. 23. Edited strains with a kinA knockout produce more CLPs than wild-type strains.

Polyketide production is shown in FIG. 24. Macrolactin production is increased in the kinA knockout strains as compared to each respective wild-type strain. Difficidin production is increased in the edited strain 7084-G77 as compared to its wild-type strain 7084.

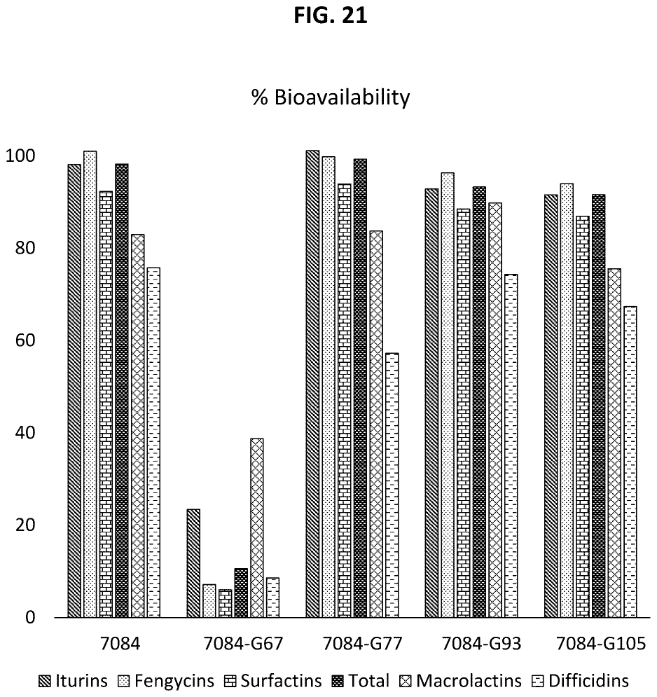

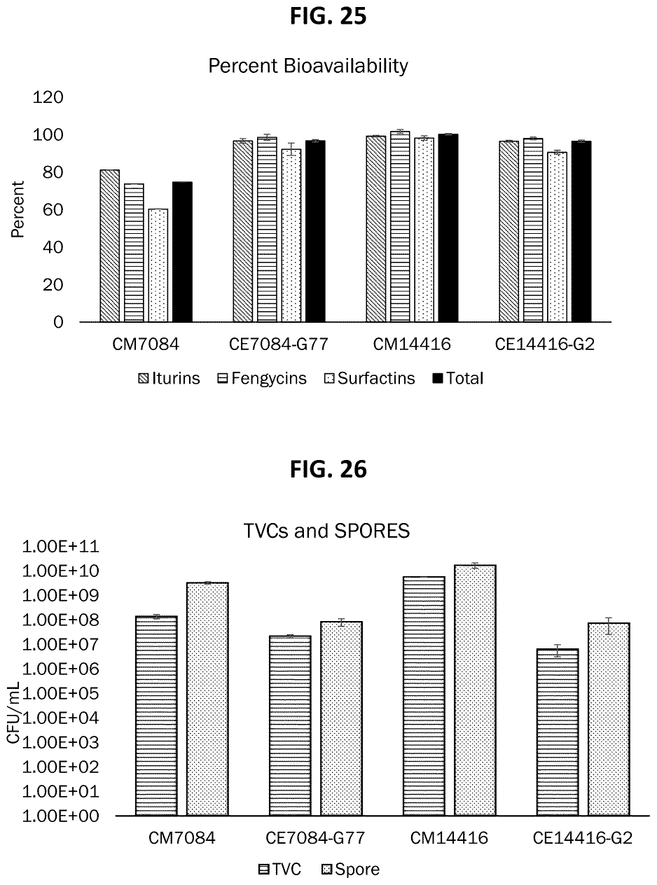

Bioavailability for the compositions were evaluated. Bioavailability is a ratio of the amount in soluble supernatant over the amount in whole cell broth (including precipitated solids). Only bioavailable compounds are responsible for activity. High bioavailability indicates all produced metabolites are able to be active in a lab/field setting. Results are shown in FIG. 21. The percent bioavailability of iturins, fengycins, surfactins, and total cyclic lipopeptides in the wild-type and kinA KO strains of Bacillus velezensis is shown in FIG. 25.

Total Viability Counts (TVC, estimates the total numbers of microorganisms) and sporulation were assessed in each of the wild-type and kinA knockout edited strains. Results are shown in FIG. 26.

A summary of 7084 genome-edited strain's composition production, as compared to the wild type strain, are given in Table 3C.

TABLE 3C
Relative Peak Areas of Secondary Metabolites

| | | (compared to WT) | | | | | |
|---|---|---|---|---|---|---|---|
| | | Cyclic Lipopeptides (CLPs) | | | Polyketides (PKs) | | |
| Strain | Edit | Iturins | Fengycins | Surfactins | Macrolactins | Difficidins | Baccilaene |
| 7084-G67 | kinA::Psfp-sfp | = | = | = | = | + | |
| High producer | | | | | | | |
| 7084-G67 | kinA::Psfp-sfp | − | − | − | − | + | |
| Low producer | | | | | | | |
| 7084-G68 | PcodY::PsigA | − | = | − | + | = | = |
| 7084-G75 | PcodY::PaprE | = | = | = | = | = | = |
| 7084-G76 | PcodY::Phag | = | = | = | = | = | = |
| 7084-G77 | ΔkinA | + | + | + | + | + | + |
| 7084-G93 | ΔthrC | = | − | = | − | + | |
| 7084-G95 | Δdif_ks1 | = | = | = | − | − | = |
| 7084-G97 | thrC::PcomA-comA | − | − | + | + | + | = |
| 7084-G103 | thrC::degU | − | − | − | − | + | − |
| 7084-G105 | thrC::Psfp-sfp | = | − | − | − | + | |

### Example 6: In Vitro Antifungal Activity of Genome Edited Microbes

Supernatant material of each culture was tested against six different fungal pathogens, as described in Table 4. Each supernatant was prepared and tested at different dilutions.

TABLE 4
Fungal Pathogens Tested

| | Spores | Incubation |
|---|---|---|
| Taxonomy | per well | time (days) |
| Fusarium gramminearum | 500 | 3-9 |
| Penicillium expansum | 500 | 3-4 |
| Fusarium oxysporum f. sp. lycopersici | 250 | 3 |
| Fusarium oxysporum f. sp. lactucae | 250 | 3-9 |
| Penicillium digitatum | 500 | 3 |
| Botrytis cinerea | 500 | 6 |
| | Inoculum Type | |
| Pythium ultimum | Agar Plug | 2-3 |

Supernatant material were obtained from each of the WI and genome-edited strains, as described above. Briefly, strains were grown in FM4 media for 4 days at 30 C. Samples were centrifuged at 15000 RPM for 20 min and supernatant was collected into a fresh tube. The respective dilutions (1:5, 1:10, 1:20, 1:50, 1:100 or 1:200) were prepared using sterile RO water

Samples were pipetted into wells of sterile 48-well plate in the BSC at 200 ul per well. 200 ul of PDA (still liquid and warm, but not hot) containing antibiotic (penn/strep) was pipetted into each well. Each plate was swirled gently while pipetting to enable the agar to mix with the supernatant and allowed up to 30 min for the agar to solidify. Appropriate amounts (5-10 ul) of fungal spore suspension was added to each well. Plates were sealed with parafilm and incubated up to 5 days. Plates were scored visually and under the microscope beginning 2 dpi (days post inoculation). Activity was scored using a rating scale of 0-5, as defined in Table 5.

TABLE 5
Antifungal Activity Categorization Scale

| Score | Description |
|---|---|
| 5 | Fungal growth completely inhibited. |
| 4 | Fungal growth heavily inhibited, very minor growth can be seen. |
| 3 | Small patches of fungus can be seen, but wells are still mostly |
| | clear. |
| 2 | Large and numerous fungal patches with pockets of inhibition are |
| | seen. |
| 1 | Very rampant fungal growth, only marginal difference from water |
| | control. |
| 0 | Fungal growth uninhibited, no difference from water control. |

Strain 7084 Wild-Type (WT) and ΔkinA (KO)

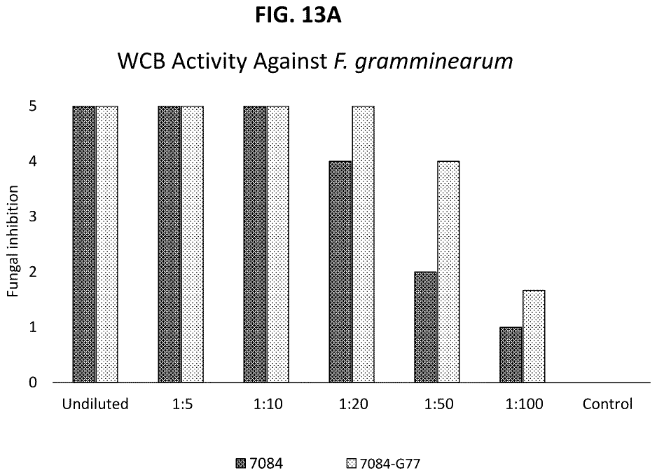

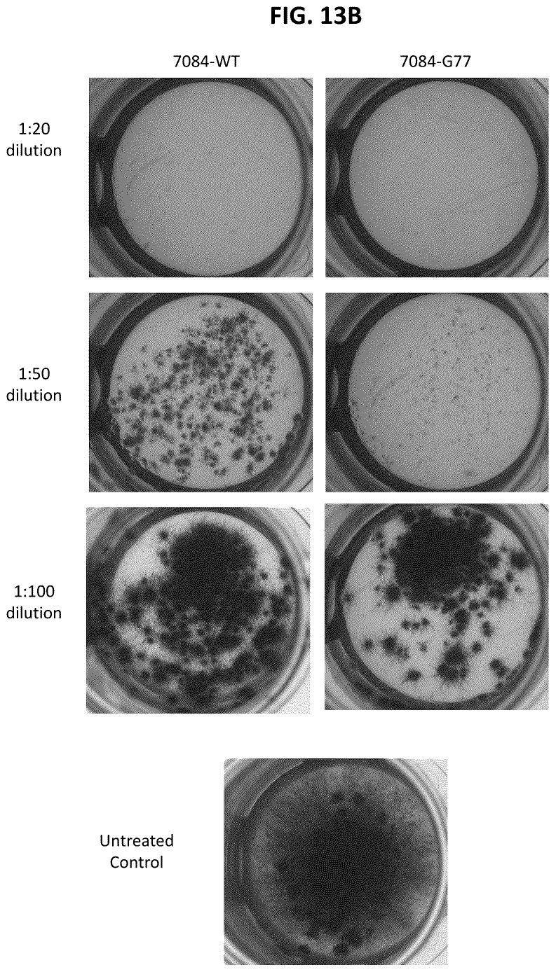

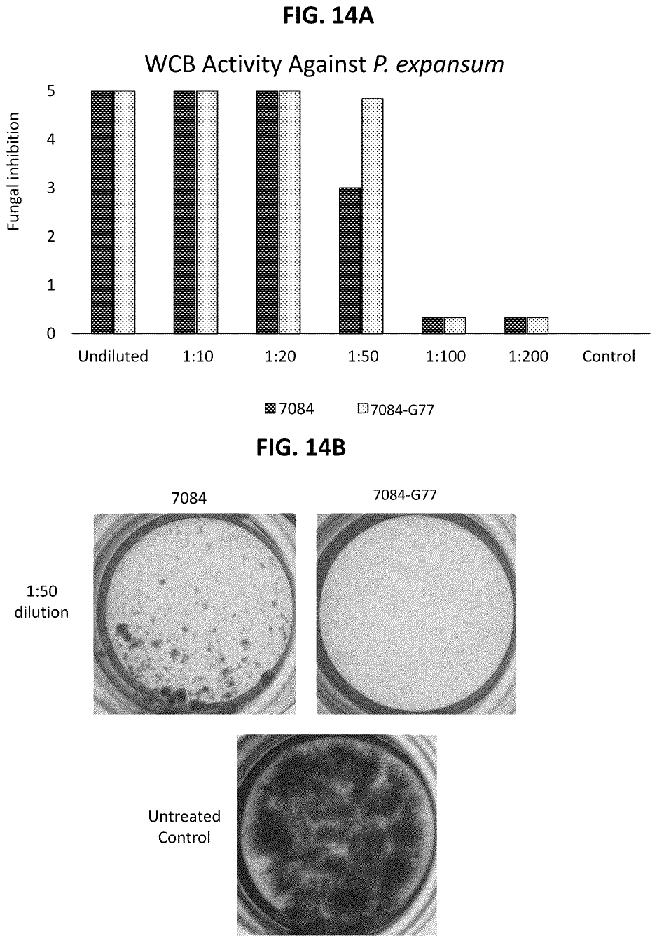

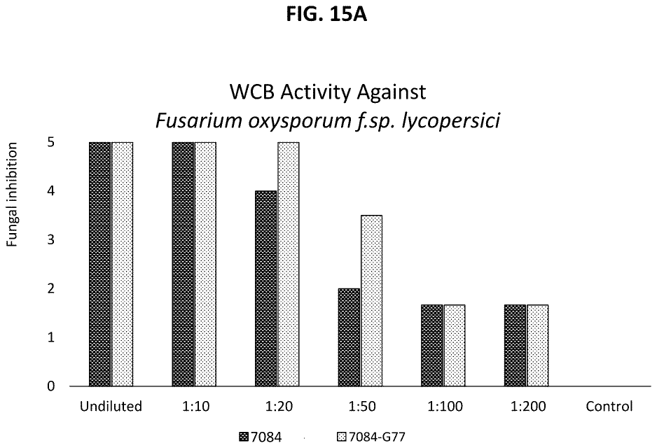

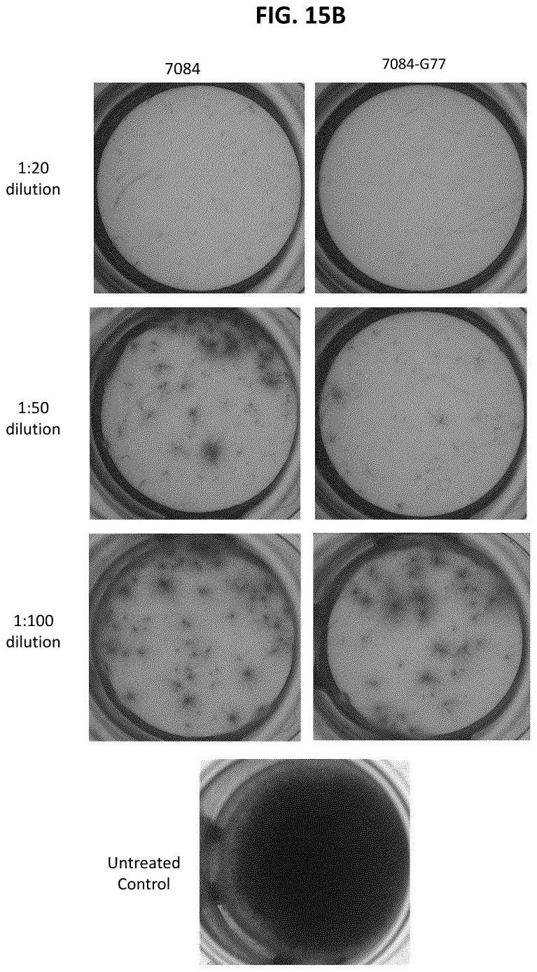

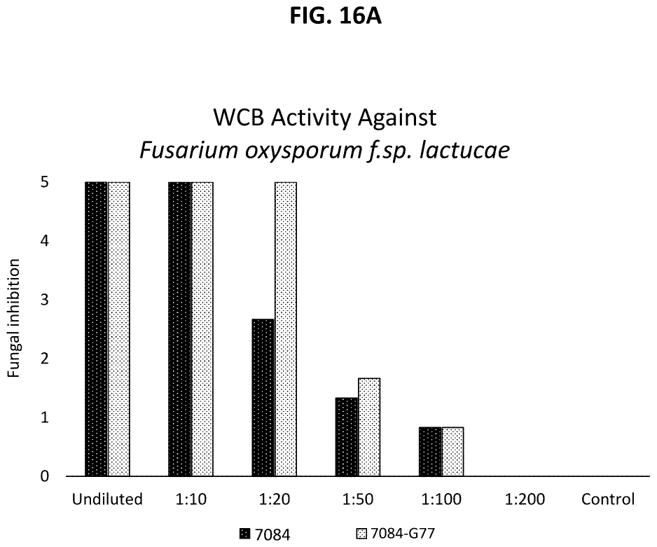

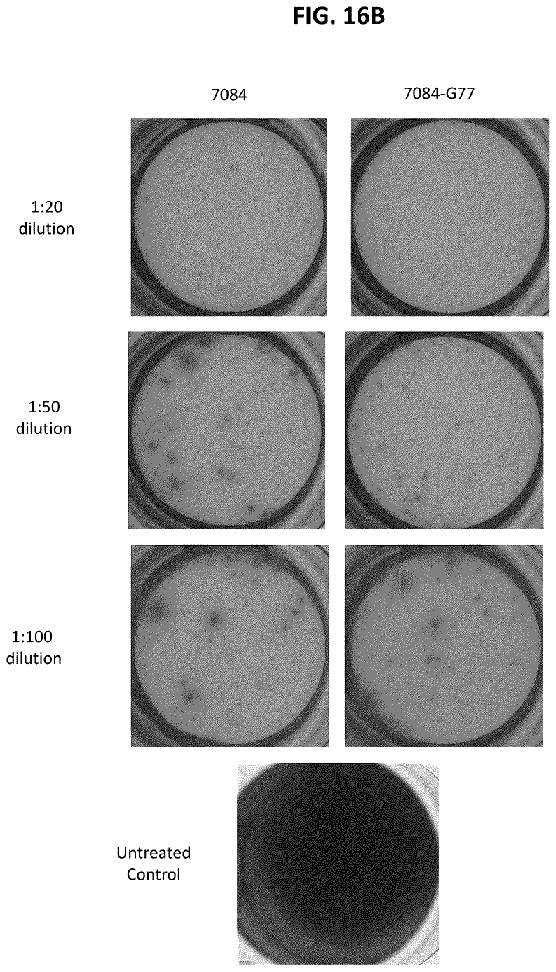

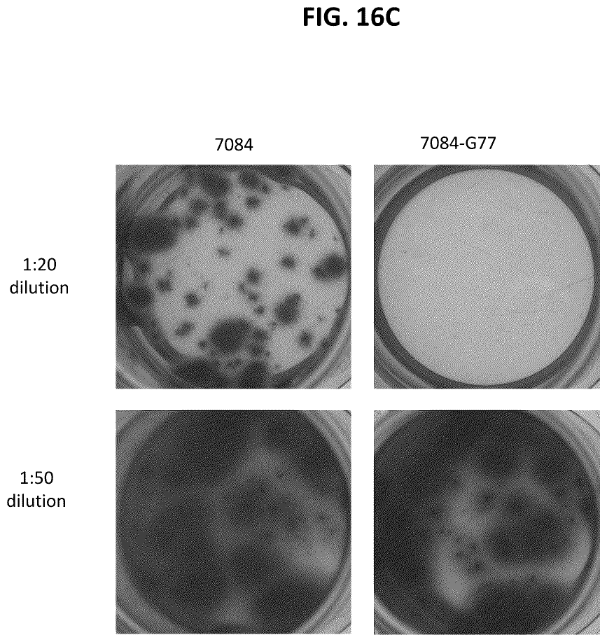

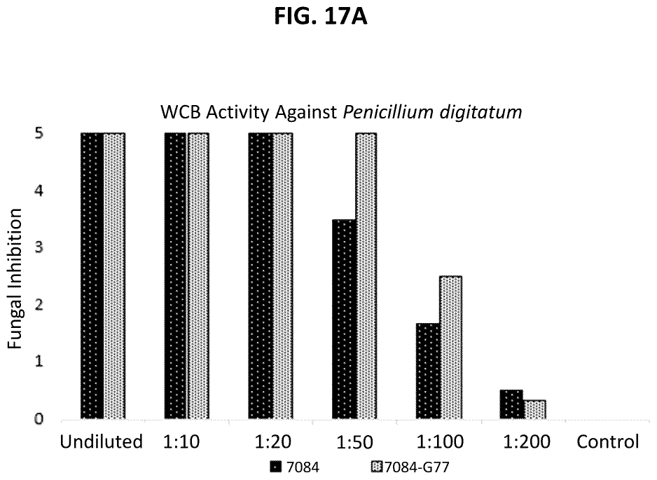

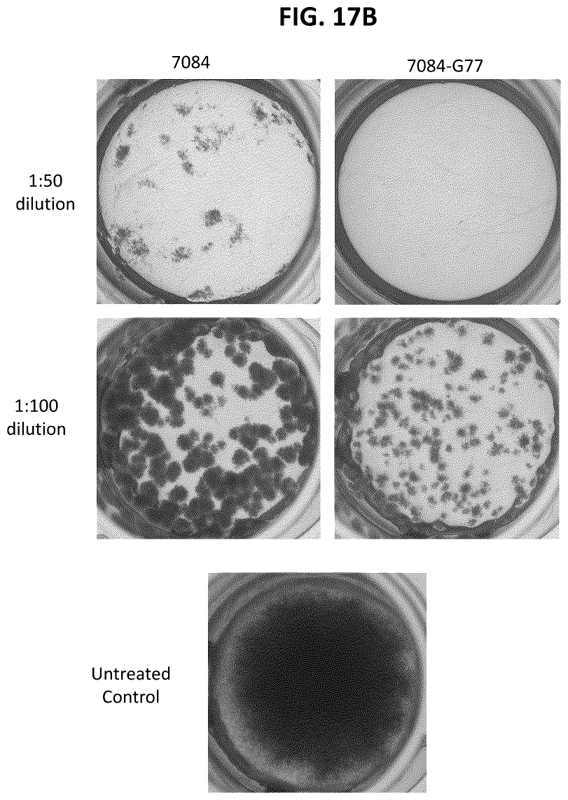

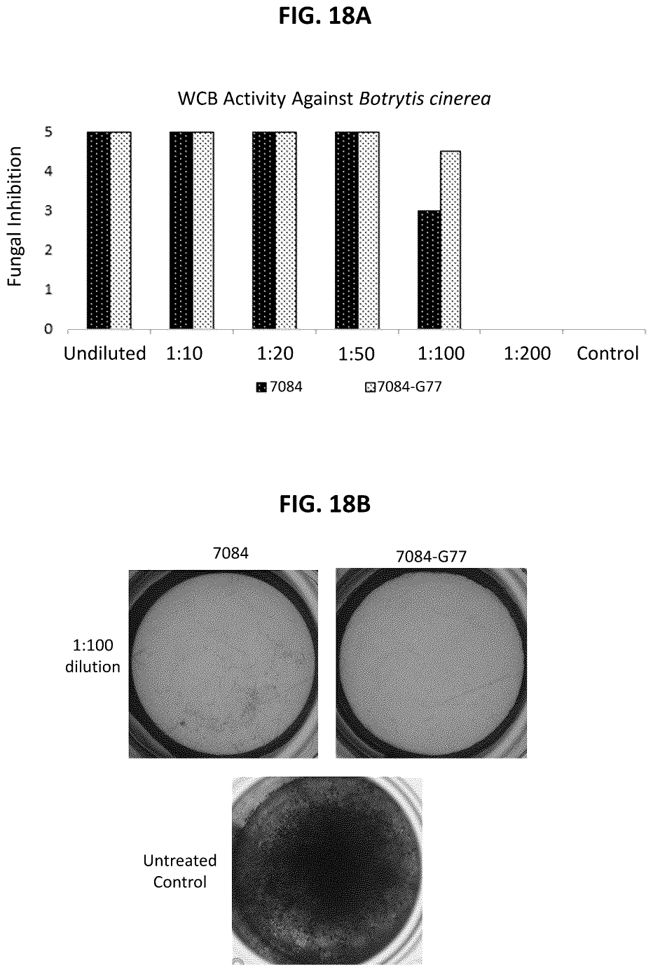

Results for WT Strain 7084, ΔkinA strain 7084-G77, and untreated controls inoculated with each pathogen, averaged across 3 wells, are given in FIGS. 13A-13B (F. gramminearum at 4 dpi (days past inoculation)), FIGS. 14A-14B (P. expansum at 4 dpi), FIGS. 15A-15B (F. oxysporum f.sp. lycopersici at 3 dpi), FIGS. 16A-16B (F. oxysporum f.sp. lactucae at 3 dpi) and FIG. 16C (F. oxysporum f.sp. lactucae at 5 dpi), FIGS. 17A-17B (Penicillium digitatum at 3 dpi), and FIGS. 18A-18B (Botrytis cinerea at 6 dpi).

The undiluted (UD), 1:5 and 1:10 diluted supernatant of CM7084 and CE7084-G77 equally inhibited growth of F. gramminearum. At 1:20, 1:50 and 1:100 dilutions, CE7084-G77 shows much stronger activity than CM7084. At 1:50, the activity of CE7084-G77 is significantly enhanced compared to CM7084.

The undiluted, 1:10 and 1:20 diluted supernatant of CM7084 and CE7084-G77 equally inhibited growth of P. expansum. At 1:50 dilution, CE7084-G77 shows enhanced activity compared to CM7084.

The undiluted, and 1:10 diluted supernatant of CM7084 and CE7084-G77 equally inhibit growth of F. oxysporum. At 1:20 and 1:50 dilutions, CE7084-G77 shows enhanced activity compared to CM7084. Activity strongly reduces at 1:100 and 1:200 dilutions, however still better than water control.

The undiluted and 1:10 diluted supernatant of CM7084 and CE7084-G77 equally inhibit growth of F. oxysporum f.sp. lactucae. At 1:20 dilution, CE7084-G77 strongly reduces hyphal growth compared to CM7084.

The undiluted, 1:10 and 1:20 diluted supernatant of CM7084 and CE7084-G77 equally inhibit growth of P. digitatum. At 1:50 and 1:100 dilutions, CE7084-G77 strongly reduces hyphal growth compared to CM7084.

The undiluted, 1:10, 1:20 and 1:50 diluted supernatant of CM7084 and CE7084-G77 equally inhibit growth of B. cinerea. At 1:100 dilution, CE7084-G77 shows enhanced activity compared to CM7084.

Strain 14416 Wild-Type (WT) and ΔkinA

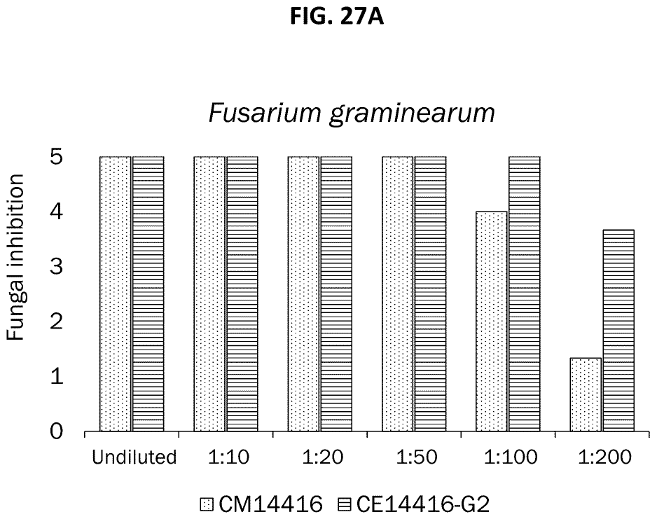

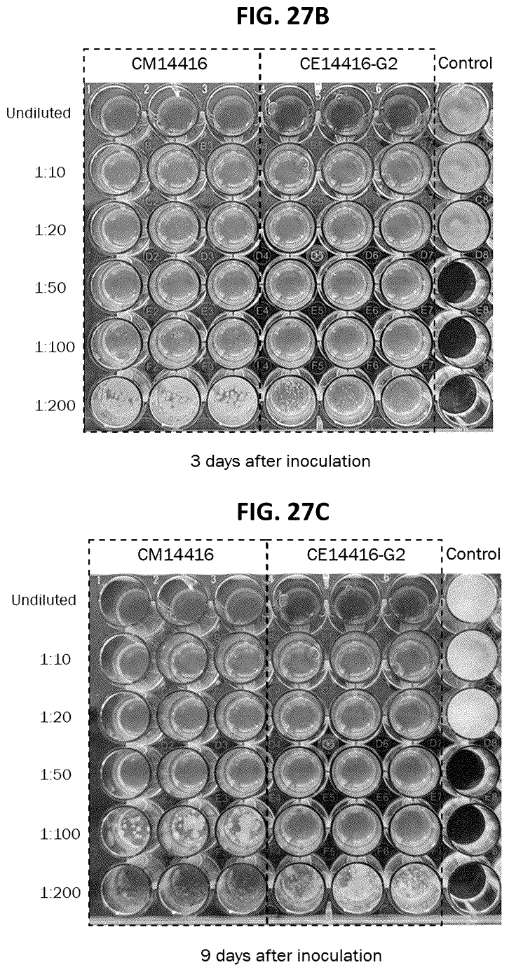

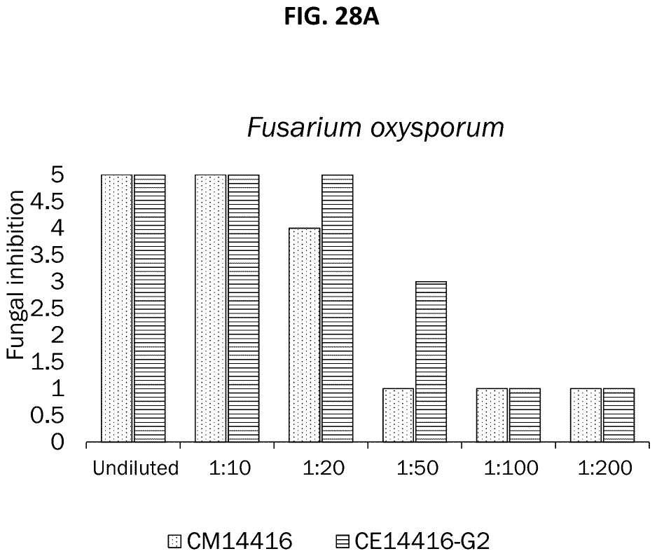

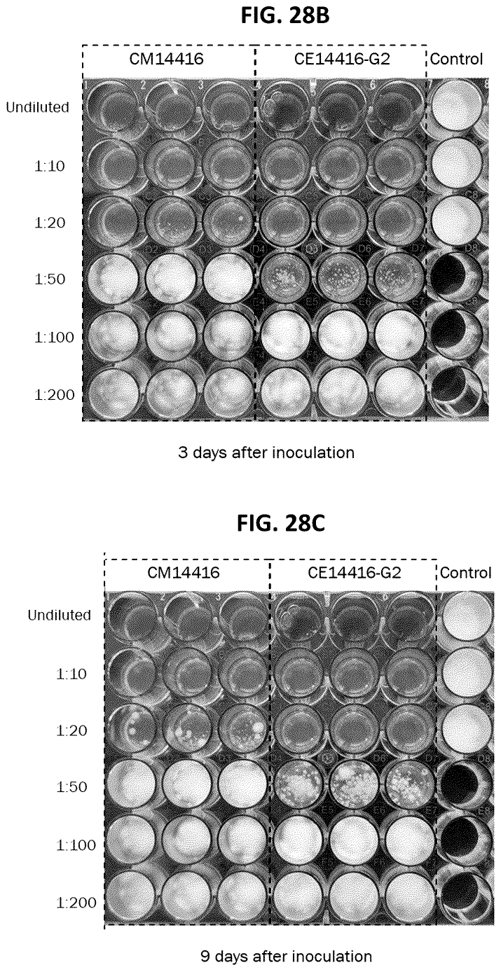

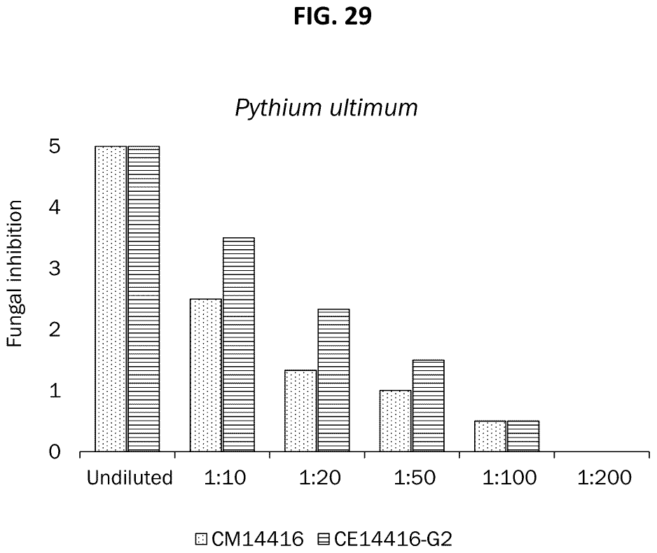

Results for WT Strain 14416, ΔkinA strain 14416-G2, and untreated controls inoculated with each pathogen, averaged across replicate wells, are given in FIGS. 27A-27C (F. gramminearum at 3 and 9 dpi (days past inoculation)), FIGS. 28A-28C (F. oxysporum at 3 and 9 dpi), and FIG. 29 (Pythium ultimum).

The undiluted, 1:10, 1:20 and 1:50 diluted supernatant of CM14416 and CE14416-G2 equally inhibited growth of F. gramminearum. At 1:100, and 1:200 dilutions, CE14416-G2 shows much stronger activity than CM14416. At 1:200, the activity of CE14416-G2 is significantly enhanced compared to CM14416.

The undiluted and 1:10 diluted supernatant of CM14416 and CE14416-G2 equally inhibited growth of F. oxysporum. At 1:100, and 1:200 dilutions, CE14416-G2 shows much stronger activity than CM14416. At 1:200, the activity of CE14416-G2 is significantly enhanced compared to CM14416.

The undiluted supernatant of CM14416 and CE14416-G2 completely inhibited hyphal growth of P. ultimum. At 1:10, 1:20 and 1:50 dilutions, CE14416-G2 reduces hyphal growth of P. ultimum better than CM14416.

Strain 102504 Wild-Type (WT) and kinA Knockout (KO)

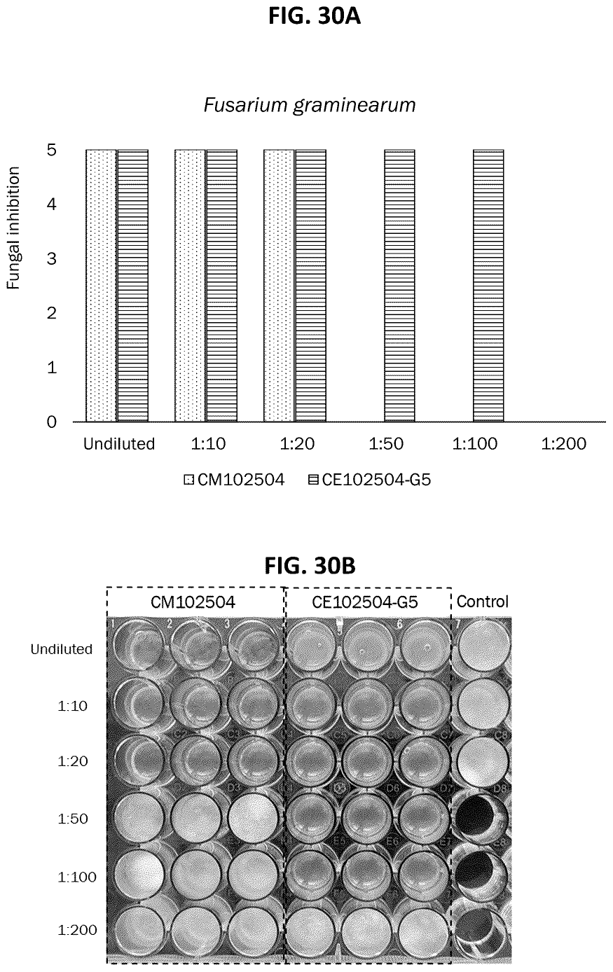

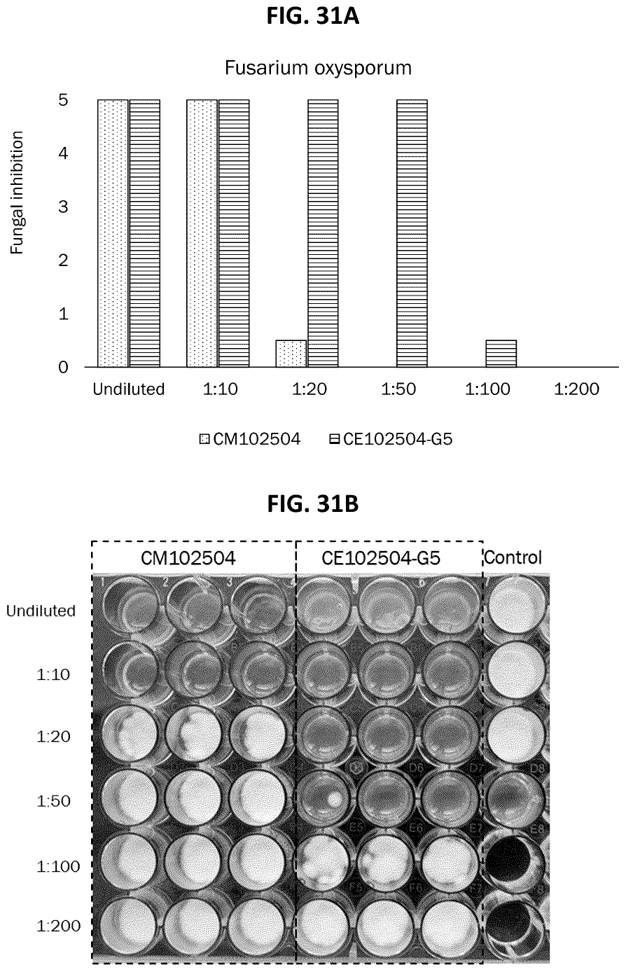

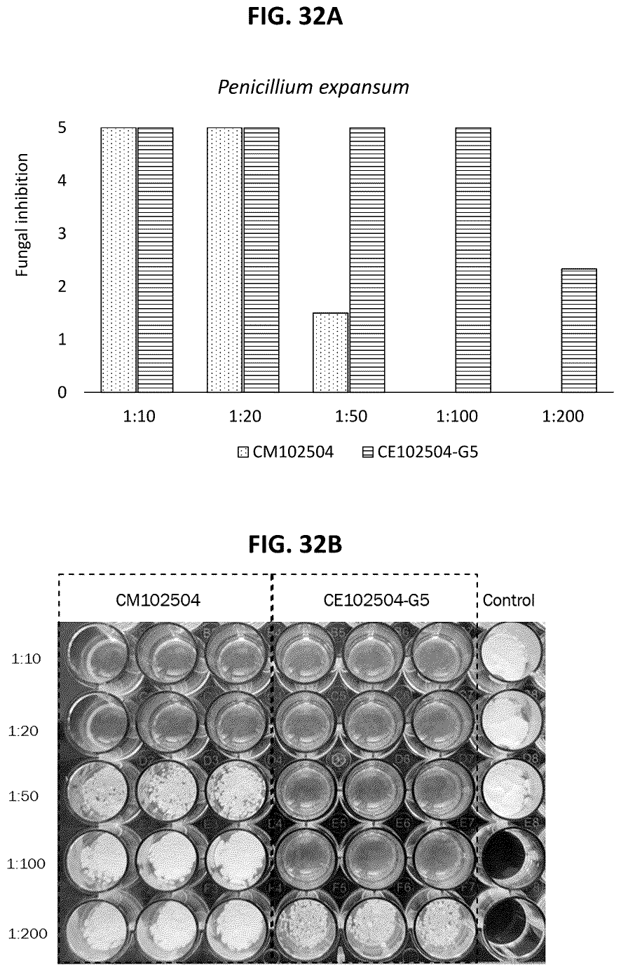

Results for WT Strain 102504, ΔkinA strain 102504-G5, and untreated controls inoculated with each pathogen, averaged across replicate wells, are given in FIGS. 30A-30B (F. gramminearum at 3 dpi (days past inoculation)), FIGS. 31A-31B (F. oxysporum at 3 dpi), and FIGS. 32A-32B (Penicillium expansum at 3 dpi).

The undiluted, 1:10, and 1:20 diluted supernatant of CM102504 and CE102504-G5 equally completely inhibited growth of F. gramminearum. At 1:50, and 1:100 dilutions, CE102504-G5 completely inhibited spore germination and hyphal growth whereas CM102504 did not. At 1:50 and 1:100 dilutions, the activity of CE102504-G5 is significantly enhanced compared to CM102504.

The undiluted and 1:10 diluted supernatant of CM102504 and CE102504-G5 equally completely inhibited growth of F. oxysporum. At 1:20, and 1:50 dilution, CE102504-G5 completely inhibited spore germination and hyphal growth whereas CM102504 did not. At 1:20 and 1:50 dilutions, the activity of CE102504-G5 is significantly enhanced compared to CM102504.

The 1:10, and 1:20 diluted supernatant of CM102504 and CE102504-G5 equally completely inhibited growth of P. expansum. At 1:50, 1:100 and 1:200 dilutions, CE102504-G5 strongly inhibited hyphal growth whereas CM102504 did not. At 1:50 and 1:100 dilutions, the activity of CE102504-G5 is significantly enhanced compared to CM102504.

kinA Knockouts in Bacillus and Paenibacillus Strains Provide Better Control of Fungal Pathogens than Wild-Type Strains.

CE7084-G77 (ΔkinA) strongly repress hyphal growth of several fungal pathogens compared to CM7084. Data suggest that the increase in CLP production in CE7084-G77 strongly translate to increase bioactivity.

CE14416-G2 (ΔkinA) strongly inhibits spore germination and hyphal growth of fungal pathogens compared to the parent strain CM14416. The results indicate that deletion of kinA in Bacillus strain CM14416 strongly increases antifungal metabolites and therefore antifungal activity against fungal pathogens.

CE102504-G5 (ΔkinA in P. polymyxa) strongly inhibits spore germination and hyphal growth of fungal pathogens compared to the parent strain CM102504. The results indicate that deletion of kinA in Paenibacillus polymyxa strain CM102504 strongly increases antifungal metabolites and therefore antifungal activity against fungal pathogens.

These results indicate that kinA regulates antifungal activity in both Bacillus and Paenibacillus genus

### Example 7: In Vitro Antibacterial Activity of Genome Edited Microbes

WT Strain 7084, GE Strain 7084-G77, and untreated controls were inoculated with the bacterial pathogen Agrobacterium tumefaciens at different dilutions, according to one of the following methods.

### Paper Disc Diffusion Assay (6 Well Plates)

Bacterial pathogen were grown (stationary) overnight in LB. The appropriate amount of pathogen culture was mixed with 0.5% top agar. 100 ul pathogen culture with 3 ml top agar was applied per standard (100 mm) petri-dish, for example 2 ml top agar per well. Just enough metabolite to soak (30 ul per disc in this assay) was applied to each disc. Discs were placed on top agar, and plates were sealed with Parafilm and incubated at 30 C.

### 96 Well-Based Assay

Bacterial pathogens were grown overnight in LB. OD was measured and concentration of pathogen for the assay was determined. 10 ul metabolite supernatant and 90 ul pathogen was applied in each well of a 96-well plate pipette. OD was measured at Time Point 0. Plates were sealed with Parafilm and incubated at 30 C for 24 hours shaking. OD was measured at Time Point 24 hours.

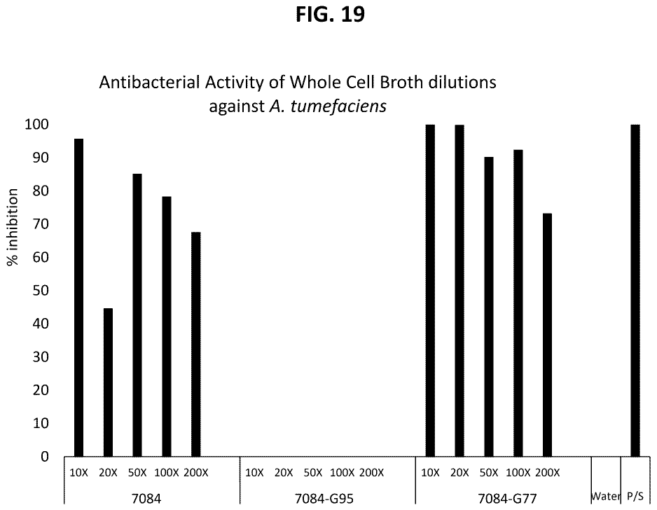

Results are shown in FIG. 19.

### Example 8: Microbe Identification and Storage

Preparation for microbe identification, and long-term storage, is performed by the following method:

### Day 1:

Use a 10 uL sterile tip to transfer a colony from a plate to a flask containing an appropriate liquid growth medium. Place the isolates on a shaker at room temperature and incubate for 2 days.

### Day 3:

Tubes may be cloudy after being on the shaker for 2 days. All samples are analyzed by PCR. Vortex each tube, collect a 50 uL sample from each vortexed tube, and dispense in a 96-well plate. Using a multichannel pipette, dispense 15 μL of the 50 uL samples into a new 96-well plate. The 96-well plate containing 35 μL of each sample will be used for phenotyping, and the 96-well plate containing 15 μL of each sample will be used for PCR analysis. 27F/1492R primers are generally used for 16S PCR analysis, as they yield better results than PB36/38. Appropriate negative controls should be included with the plate and analyzed by PCR. The plate will be analyzed by PCR using an Eppendorf thermocycler. Once the PCR is finished, run a gel using standard gel electrophoresis techniques. This is important because most isolates are grown enough where they should ideally be put into long-term storage on day 3. The PCR and gel electrophoresis analysis are used to confirm that the isolates contain bacteria, rather than other microbes. For isolates that do not pass PCR or have clear broth, vortex tubes and use a loop to streak out onto a petri plate. Check after several days to see if anything grows, or if the tube is contaminated. For isolates that pass PCR, dispense 600 μL of 50% glycerol into a 2 ml screw cap tubes and add 1200 μL of the bacterial culture, such that the broth is stored in 20% glycerol. Store the glycerol stock at −80° C. and record an image of the gel of the PCR samples.

### Day 4:

Check the petri plates of the streaked isolates that failed PCR for growth. (During this time, the 2 ml broth tubes will remain on the shaker.) Once there is growth on the plate and the colonies appear to have been successfully isolated, dispense 600 μL of the broth-glycerol mixture in the small tube, and put both tubes in their respective −80 C boxes. It is possible for isolates to fail the PCR check because of any of the following reasons: the primers may not work on all bacteria, the isolate is actually a fungus, the isolate is very adherent and therefore does not homogenize in the broth, the isolate produces too much EPS therefore needs dilution prior to PCR set-up, or the isolate is a slow grower. Over the next few days, continue checking the plate to confirm that only a single bacterial species was isolated. If contamination is observed, prepare a new isolate. The viability of the prepared glycerol stocks should be verified.

### Example 9: Formulation of Microbes

Microbes identified according to the previous examples may be formulated with additional components for application via methods such as, but not be limited to: seed treatment, root drench, root wash, seedling soak, foliar application, soil inocula, in-furrow application, sidedress application, soil pre-treatment, wound inoculation, drip tape irrigation, vector-mediation via a pollinator, injection, osmopriming, hydroponics, aquaponics, aeroponics. The formulation comprising the microbes are prepared for agricultural application as a liquid, a solid, or a gas formulation. Application to the plant is achieved, for example, as a powder for surface deposition onto plant leaves, as a spray to the whole plant or selected plant element, as part of a drip to the soil or the roots, or as a coating onto the plant element prior to planting. Such examples are meant to be illustrative and not limiting to the scope of the invention.

Media components for an exemplary microbe preparation are shown below. Add all contents with 50% of the final volume of water needed, and stir the solution at an elevated temperature until dissolved. After all contents have been dissolved, use sterile RO water to bring the solution to the final desired volume. Field trial preparations are typically performed using the 4× formulation.

TABLE 6
Exemplary media components and concentrations for microbe formulation

| | | | | fill vol. | | | | fill vol. |
|---|---|---|---|---|---|---|---|---|
| | 0.9X | fill vol. | 1.1x | below | 2x | fill vol. | 4x | below |
| | 1 ml | below | 30 ml | 30 | 1 ml | below | 1 ml | 10 |
| Xanthan Gum (mg) | 1.8 | 0 | 2.2 | 66 | 4 | 0 | 8 | 80 |
| Trehalose (mg) | 45.3 | 0 | 55 | 1650 | 100 | 0 | 200 | 2000 |
| Isomalt (mg) | 22.65 | 0 | 27.5 | 825 | 50 | 0 | 100 | 1000 |

TABLE 7
Exemplary media components for microbe formulation

| Component | CAS# |
|---|---|
| Sucrose | 57-50-1 |
| Proflo | 68308-87-2 |
| Yeast extract | 8013-01-2 |
| Tryptone | 91079-40-2 |
| MgSO4•7H2O | 10034-99-8 |
| KH2PO4 | 7778-77-0 |
| K2HPO4 | 7758-11-04 |
| NaNH4HPO4•4H2O | 7783-13-3 |
| CaCl2 | 10043-52-4 |
| MnSO4•1H2O | 10034-96-5 |
| ZnSO4•7H2O | 7446-20-0 |
| CuSO4•5H2O | 7758-99-8 |
| Na2MoO4*2H20 | 10102-40-6 |
| FeSO4*7H20 | 7782-63-0 |

TABLE 8
Exemplary media components for microbe formulation

| Component | CAS# |
|---|---|
| Tryptone | 91079-40-2 |
| Soy peptone | 91079-46-8 |
| NaCl | 7647-14-5 |
| K2HPO4 | 7758-11-4 |
| Glucose | 50-99-7 |

TABLE 9
Exemplary media components for microbe formulation

| Component | CAS# |
|---|---|
| Trehalose | 6138-23-4 |
| Isomalt | 64519-82-0 |
| Xantham gum | 11138-66-2 |

The procedure to mix TIX formulation is as follows: Measure all dry ingredients into a 50 ml tube. Vortex the ingredients well to ensure xanthan gum is “separated” through the other carbon sources. Add about half of total sterile RO water to the mix, vortex. Use the long end of an L-spreader to break up chunks as much as you can. Heat some sterile RO water in the microwave to warm water bath temperature (45-50° C.). Add the remaining sterile RO water to the mix, vortex. Repeat step 4 and vortex as needed until you have a clear solution with no lumps. Spin down the bubbles created in the process of mixing by using a centrifuge for 5-10 seconds on “fast spin”. Remember to have a balance to counter the formulation (TIX) tube. Allow formulation to cool to room temperature. 1. Mix in the microbial consortia. Vortex to ensure homogeneity. It is ideal to add microbes at a concentration of 1×10\hat{ }9 CFU/ml to the formulation.

Apply the formulation to the plant or plant element for testing in field trial.

For foliar applications, microbial consortia is combined with appropriate spray adjuvant and diluted in spray volume water.

### Example 10: Application of Microbes to Plant Elements and Cultivation Thereof

A microbial composition (comprising one or more isolated microbes of a single strain, a consortium, a community, a combination, or any combination of the preceding) is prepared according to the previous Examples. The microbial composition comprises one or more microbes, optionally in combination with one or more additional microbes disclosed herein.

### Microbial Compositions for Application

In some methods, the microbial composition is dried and applied directly to a plant element.

In some methods, the microbial composition is suspended in a liquid formulation for application to a plant element.

In some methods, the microbial composition is combined with another composition, such as but not limited to: a carrier, a wetting agent, a stabilizer, a salt. In some methods, the other composition comprises a molecule that introduces additional agriculturally-beneficial outcomes to the plant to which the microbial composition is applied. The other composition includes, for example but not limited to: an herbicide, a fungicide, a bactericide, a pesticide, an insecticide, a nematicide, a biostimulant.

### Application Types

The microbial composition is applied to a plant element, at a time during development appropriate to the desired outcome, for example: in a formulation of a pre-planting soil drench/in-furrow application; as a seed or other reproductive element treatment; as a post-planting reproductive element application; as an in-furrow, drip, or drench application after planting; as a direct application to a plant element (e.g., root, leaf, stem); as an application to a harvested plant element (e.g., a fruit or a grain). Combinations of application types are also tested.

### Application Methods

The microbes (wild-type and/or edited) described herein may be artificially associated with a heterologous composition (e.g., a composition with which it is not naturally associated either by type or by concentration or both) in a number of ways. In some aspects, a microbial colony is selected for scale-up fermentation to produce a larger amount of the microbe. In some aspects, a slurry of microbe and fermentation media is associated with the heterologous composition. In some aspects, a whole cell broth is associated with the heterologous composition. In some aspects, a supernatant is associated with the heterologous composition. In some aspects, a substantially cell-free preparation is associated with the heterologous composition. By the phrase “associated with”, it is intended that the microbe or preparation therefrom is introduced to the heterologous composition in such a manner that creates a new composition by virtue of the combination of the microbe/preparation and the heterologous composition.

A substantially cell-free formulation (i.e., the majority of the formulation is free of cells) of the microbial composition can be prepared by centrifugation, autoclaving, sterile filtration, acid extraction, in-line sterilization, acid extraction or a combination of these methods. Lack of living cells is confirmed by serially diluting the cell-free formulation and plating on multiple agar types. Absence of colonies confirms cell-free status.

The microbial composition is applied to (inoculating) a plant or plant element or plant product (pre-planting, post planting, pre-harvest, or post-harvest). This can be accomplished, for example, by applying the agricultural composition to a hopper or spreader or tank, which contains the microbial composition and which is configured to broadcast the same.

A seed coating of the microbial composition is applied to one or more seeds of a crop plant. Upon applying the isolated microbe as a seed coating, the seed is planted and cultivated according to practices established for that crop.

Alternatively, the microbial composition is applied to the soil for the benefit of a plant existing in that soil. Methods of soil application include in-furrow treatment, drench, and drip applications.

Alternatively, the microbial composition is applied to the surface of a plant or plant part after germination.

Alternatively, the microbial composition is applied to material obtained from the plant after harvest.

A control plot of plants, which did not have the isolated microbe applied, are also planted. Plants associated with the microbial composition exhibit improved characteristics of interest.

Application methods may be performed according to any protocol known in the art.

Plant elements, plants, or growth medium (e.g., soil) may further be inoculated with a disease or pest, according to the purpose of the test.

An exemplary, non-limiting protocol for drenching tomato plants is given below:
1. Ten days after planting carefully separate plants out into 6 reps for each treatment. Plants are delicate and leaves can tear easily. Ensure that the size and overall appearance of plants is as uniform as possible (The purpose of thinning is continuing with an homogenous plant population). Transplant if there are not enough plants per reps. See step 3 for guidelines on transplanting.
2. Begin thinning pots down to one plant per pot. Remove the smaller plant, one that is unhealthy or deformed in some way. If there are 2 or more healthy plants per pot, the extras can be transplanted into another pot. Use leftover soil prepped from initial planting or from pots where seeds did not germinate.
3. To transplant: If some pots didn't germinate, they can be filled with a plant from another container. To do this simply scoop out the extra plant (trying to scoop out as much root mass as possible without disturbing the other plant) with a scoopula and place into a hole made in the empty pot. Firm the soil around the plant with slight finger pressure.
4. Space out the pots into 6 pot lines (1 line of pots per treatment), will take 4 RL98 trays. Once done, have a look at all the treatments and consider making some pot switches to ensure some treatments don't have all large plants and others have all large plants.
5. Change gloves if necessary. Label each pot with your pre-prepared Avery Labels. Treatments should be labeled into rows of 6 replicates i.e., 1-1, 1-2, 1-3 to 1-6, etc. Makes it easier to find all replicates for each treatment.
6. Two weeks after planting (roughly 4 days after thinning and labeling), obtain treatments from the Microbiology team; set on the table with trays of prepped plants. Gather combitips, repeater, and RO water. (note: Plants should be watered lightly the day of treatment).
7. Mix microbial solution by inverting tube/container (microbial treatment) 2-3 times or give a light shake. Set combitip to dispense 2 ml. Collect treatment fluid into combitip, dispense first step back into the tube. Ensure the treatment you have corresponds to the row of plants to be treated. Once confirmed, gently dispense 2 ml of treatment onto the surface soil of each pot, close to the stem but avoid direct contact with the stem and leaves.
8. Dispose of combitip and repeat step 6 for all treatments. For the inoculated control (IC or InoCon) and untreated control (UTC), apply RO water in place of a treatment. Once all treatments have been applied, place plants back into growth chamber for (optional inoculation), growth, evaluation.
   Visualization of Microbes Associated with Plant Elements

Individual microbes can be tagged with a fluorescent protein according to methods known in the art. Microscopic image analysis demonstrated that the microbes disclosed herein were found associated with various plant tissues.

1. A synthetic composition comprising a genetically engineered microbe, whole cell broth therefrom, and/or an exudate therefrom, wherein said genetically engineered microbe comprises at least one genetic mutation at least one locus within its genome, wherein said genetic mutation alters the production of one or more secondary metabolite, and wherein said genetic mutation imparts the capability of control of at least one pathogen to the genetically engineered microbe; wherein the at least one locus is selected from the group consisting of: kinA, thrC, and any plurality and/or combination of the preceding; wherein the genetic mutation is a knockout, knock-in, disruption, insertion of at least one nucleotide, deletion of at least one nucleotide, replacement of at least one nucleotide, chemical modification of at least one nucleotide, molecular structure alteration of at least one nucleotide, downregulation of at least one gene, upregulation of at least one gene, and/or any combination and/or plurality of the preceding.

2. (canceled)

3. (canceled)

4. The synthetic composition of claim 1, wherein the non-genetically engineered microbe from which the genetically engineered microbe was obtained is of the genus Bacillus or the genus Paenibacillus.

5. (canceled)

6. The synthetic composition of claim 1, wherein the secondary metabolite is a cyclic lipopeptide or a polyketide.

7. The synthetic composition of claim 2, wherein the cyclic lipopeptide is selected from the group consisting of: an Iturin, a Fengycin, a Surfactin, any plurality of the preceding, and any combination of the preceding.

8. The synthetic composition of claim 2, wherein the polyketide is selected from the group consisting of: a Macrolactin, a Difficidin, a Baccilaene, any plurality of the preceding, and any combination of the preceding.

9. The synthetic composition of claim 1, wherein the pathogen is a fungus.

10. The synthetic composition of claim 9, wherein the fungus is of a genus selected from the group consisting of: Fusarium, Penicillium, Botrytis, and Pythium.

11. (canceled)

12. (canceled)

13. A formulation comprising the synthetic composition of claim 1.

14. The formulation of claim 13, wherein the synthetic composition is substantially purified.

15. The formulation of claim 13, wherein the composition of (b) further comprises one or more formulation components selected from the group consisting of: a salt, a binder, a surface-active agent, a surfactant, a wetting agent, a dispersing agent, an emulsifying agent, a solubilizing agent, an organic solvent, a gelling agent, a thickening agent, an anti-settling agent, a preservative, a stabilizer, an anti-freezing compound, a plurality of any of the preceding, and any combination of the preceding.

16. A method of controlling a pathogen, said method comprising applying to an object the synthetic composition of claim 1.

17. The method of claim 16, wherein the pathogen is a bacterium.

18. The method of claim 16, wherein the pathogen is selected from the group consisting of: a fungus, a bacterium, and/or any combination and/or plurality of the preceding.

19. The method of claim 18, wherein the fungus is of a genus selected from the group consisting of: Fusarium, Penicillium, Botrytis, and Pythium

20. The method of claim 16, wherein the synthetic composition further comprises one or more agents selected from the group consisting of: a pesticide, an herbicide, a bactericide, a fungicide, an insecticide, a virucide, a miticide, a nematicide, an acaricide, a plant growth regulator, a rodenticide, an anti-algae agent, a biocontrol agent, a fertilizer, a biopesticide, a biostimulant, and any combination and/or plurality of the preceding.

21. The method of claim 16, wherein the object is a plant or plant part.

22. The method of claim 21, wherein the plant part is a root, leaf, stem, flower, seed, bulb, or fruit.

23. The method of claim 16, wherein the composition of (b) further comprises one or more formulation components selected from the group consisting of: a salt, a binder, a surface-active agent, a surfactant, a wetting agent, a dispersing agent, an emulsifying agent, a solubilizing agent, an organic solvent, a gelling agent, a thickening agent, an anti-settling agent, a preservative, a stabilizer, an anti-free compound, a plurality of any of the preceding, and any combination of the preceding.

24. The method of claim 16, wherein the composition further comprises one or more additional agents selected from the group consisting of: a pesticide, an herbicide, a bactericide, a fungicide, an insecticide, a virucide, a miticide, a nematicide, an acaricide, a plant growth regulator, a rodenticide, an anti-algae agent, a biocontrol agent, a fertilizer, a biopesticide, a biostimulant, and any combination and/or plurality of the preceding.

25. The method of claim 21, wherein the synthetic composition, whole cell broth, or exudate is applied to the plant or plant part prior to germination.

26. The method of claim 21 wherein the synthetic composition, whole cell broth, or exudate is applied to the plant or plant part post-harvest.

27. The method of claim 21, wherein the synthetic composition, whole cell broth, or exudate is applied to the plant or plant part during the vegetative phase of the plant.

28. The method of claim 21, wherein the synthetic composition, whole cell broth, or exudate is applied to the plant or plant during the reproductive phase of the plant.

29. The method of claim 21, wherein the synthetic composition, whole cell broth, or exudate is applied to the plant or plant part pre-harvest.

30. The method of claim 21, wherein the plant or part therefrom is cultivated in a field, harvested, placed in storage, and/or distributed.

31. The method of claim 21, further comprising applying a plurality of compositions to the plant or plant part.

32. A method of reducing or inhibiting hyphal growth of a fungus, the method comprising:
a. modifying a strain of Bacillus or Paenibacillus by knocking out, reducing, and/or downregulating the activity of a kinA gene, to create a modified strain,
b. introducing the modified strain to a fungus.

33. A method of reducing or inhibiting spore germination of a fungus, the method comprising:
a. modifying a strain of Bacillus or Paenibacillus by knocking out, reducing, and/or downregulating the activity of a kinA gene, to create a modified strain,
b. introducing the modified strain to a fungus.

34. A synthetic composition comprising:
a. a plant or plant part, and
b. the synthetic composition of claim 1.

35. The synthetic composition of claim 34, wherein the synthetic composition is substantially confined within an object selected from the group consisting of: bottle, jar, ampule, package, vessel, bag, box, bin, envelope, carton, container, silo, shipping container, truck bed, and case.

36. A plurality of synthetic compositions of claim 34, wherein individuals of the plurality are placed in a growth medium.

37. The plurality of synthetic compositions of claim 36, wherein the growth medium is soil.
